(19) United States
(12) Patent Application Publication
(10) Pub. No.: US 2016/0219881 A1
(43) Pub. Date: Aug. 4, 2016

(54) HERBICIDALLY ACTIVE 2-HALOGEN-4-ALKYNYL-PHENYL-PYRAZOLIDINE-DIONE OR PYRROLIDINE-DIONE DERIVATIVES

(71) Applicant: SYNGENTA LIMITED, Guildford (GB)

(72) Inventors: Shuji HACHISU, Bracknell Berkshire (GB); James Nicholas SCUTT, Bracknell Berkshire (GB); Nigel James WILLETTS, Bracknell Berkshire (GB)

(73) Assignee: Syngenta Limited, Guildford (GB)

(21) Appl. No.: 15/022,416

(22) PCT Filed: Sep. 18, 2014
(86) PCT No.: PCT/EP2014/069897

§ 371 Date: Mar. 16, 2016

(30) Foreign Application Priority Data
Sep. 20, 2013 (GB) 1316717.6
Feb. 3, 2014 (GB) 1401784.2

(51) Int. Cl. A01N 43/90; A01N 43/40; C07D 487/04; C07D 231/34; C07D 491/107; C07D 231/36; C07D 498/04; C07D 207/36; A01N 43/36; C07D 401/06; A01N 43/50

(52) U.S. Cl. CPC A01N 43/90; C07D 401/06; A01N 43/40; C07D 487/04; C07D 231/34; A01N 43/50; C07D 231/36; C07D 498/04; C07D 207/36; A01N 43/36; C07D 491/107

(57) ABSTRACT

The present invention relates to a compound of formula (I), wherein CKE is of sub-formula (A) and/or (B), wherein: R¹ is methyl or methoxy; R² is hydrogen, methyl, ethyl, n-propyl, n-butyl, cyclopropyl, ethynyl, cyano, C₁-C₃alkoxy, C₁-C₂fluoroalkoxy, (difluoro)vinyloxy, C₁-C₂alkoxy-C₁-C₃alkoxy-, or C₁fluoroalkoxy-C₁C₃alkoxy-; and X is NR⁴ or CR⁵R⁶; wherein R3, R4, R5 and R6 are as defined herein; wherein the compound of formula (I) is optionally present as an agrochemically acceptable salt thereof. These compounds are suitable for use as herbicides. The invention therefore also relates to a method of controlling weeds, especially grassy monocotyledonous weeds, in crops of useful plants, comprising applying a compound of formula (I), or a herbicidal composition comprising such a compound, to the plants or to the locus thereof.

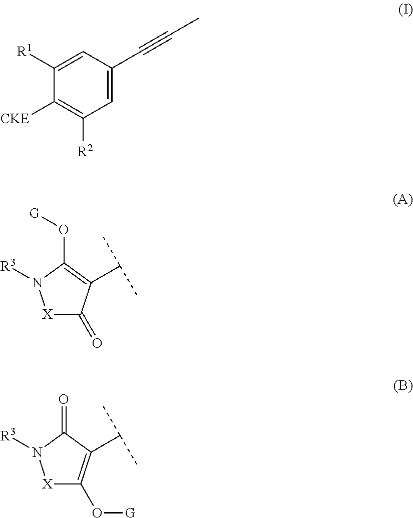

The present invention relates to herbicidally active heterocyclic diones, in particular pyrazolidine-dione (more particularly pyrazolidine-3,5-diones) or pyrrolidine-dione (more particularly pyrrolidine-2,4-dione) compounds or derivatives thereof (e.g. enol ketone tautomer derivatives thereof), to processes for their preparation, to herbicidal compositions comprising those compounds, and to their use in controlling weeds such as grassy monocotyledonous weeds, especially in crops of useful plants, or in inhibiting undesired plant growth.

EP 0 456 063 A2 discloses 3-(substituted-phenyl)-pyrrolidine-2,4-dione derivatives, and their use as insecticides, acaricides and herbicides. EP 0 355 599 A1 discloses fused 3-(substituted-phenyl)-pyrrolidin-2,4-dione derivatives, and their use as herbicides, fungicides, antimycotics, insecticides and acaricides.

WO 98/05638 A2 discloses phenyl-substituted heterocyclic keto-enols or derivatives thereof, for example 3-(2- or 2,5-substituted-phenyl)-pyrrolidin-2,4-dione derivatives, and their use as pesticides.

WO 92/16510 A1 discloses 4-aryl-pyrazolidine-3,5-dione herbicides, and their use as herbicides, acaricides and insecticides. EP 0 508 126 A1 discloses 3-hydroxy-4-aryl-5-oxo-pyrazoline derivatives, and their use as insecticides, acaricides and herbicides. WO 96/21652 A1 discloses 4-aryl- and 4-heteroaryl-5-oxopyrazoline derivatives having pesticidal properties.

WO 99/47525 A1 discloses herbicidally active 3-hydroxy-4-aryl-5-oxypyrazoline derivatives. In WO 99/47525 A1, the 3-hydroxy-5-oxy-pyrazoline ring system is fused, through the two ring-nitrogens of the pyrazole, to a further ring which also contains a ring-oxygen atom.

M. Muehlebach et al., Bioorganic and Medicinal Chemistry, 2009, 17, 4241-4256, discloses a class of 3-hydroxy-4-phenyl-5-oxo-pyrazolines, in particular aryl (here, phenyl) diones incorporating a [1,4,5]oxadiazepane ring, and most particularly the cereal herbicide pinoxaden.

WO 01/17972 A2 discloses phenyl-substituted (such as 4-methyl-2,6-diethyl-phenyl-substituted) carbocycles or heterocycles suitable for use as herbicides.

WO 01/74770 discloses C₂-phenyl-substituted cyclic ketoenols and their use as pesticides and herbicides.

WO 03/013249 A1 discloses selective herbicidal compositions comprising (a) a (substituted-phenyl)-substituted cyclic ketoenol and (b) a compound which improves crop plant compatibility, in particular cloquintocet-mexyl or mefenpyr-diethyl. In WO 03/013249 A1 the cyclic ketoenol (whose tautomer is a cyclic dione) can in particular be a 3-(substituted-phenyl)-pyrrolidine-2,4-dione, a 3-(substituted-phenyl)-tetrahydrofuran-2,4-dione, a 3-(substituted-phenyl)-pyran-2,4-dione derivative, a 2-(substituted-phenyl)-cyclopentane-1,3-dione, or a 2-(substituted-phenyl)-cyclohexane-1,3-dione, et al., or a derivative (e.g. ester or carbonate derivative) of these cyclic ketoenols/cyclic diones.

WO 2007/068427 A2 discloses a composition comprising (a) a (substituted-phenyl)-substituted cyclic ketoenol as a herbicide, and (b) an ammonium and/or phosphonium salt allegedly to boost activity. In WO 2007/068427 A2, the cyclic ketoenol (whose tautomer is a cyclic dione) can in particular be a 3-(substituted-phenyl)-pyrrolidine-2,4-dione, a 3-(substituted-phenyl)-tetrahydrofuran-2,4-dione, a 3-(substituted-phenyl)-pyran-2,4-dione derivative, a 2-(substituted-phenyl)-cyclopentane-1,3-dione, or a 2-(substituted-phenyl)-cyclohexane-1,3-dione, a 4-(substituted-phenyl)-pyrazolidine-3,5-dione, et al., or a derivative (e.g. ester or carbonate derivative) of these cyclic ketoenols/cyclic diones.

WO 2006/089633 A2 discloses spiroketal-substituted heterocyclic ketoenols, more particularly 5-spirocyclic-3-(substituted-phenyl)-pyrrolidine-2,4-diones or tetrahydrofuran-2,4-diones, or derivatives thereof, and their use as pesticides, microbicides and/or herbicides.

WO 2007/121868 A1 discloses 5-[alkoxyalkyl- or heterocyclyl(alkyl)-]-substituted-3-(substituted-phenyl)-pyrrolidine-2,4-diones, or derivatives thereof, and their use as pesticides, microbicides and/or herbicides.

WO 2012/175666 A1 discloses certain substituted N-oxy pyrazolo-triazepine-dione derivatives having pesticidal and herbicidal properties.

WO 2011/151199 A1 discloses new 5-spiroheterocyclic-3-(substituted-phenyl)-pyrrolidine-2,4-dione compounds, or derivatives, in which the phenyl has at least one C₂-C₆alkenyl, C₂-C₆alkynyl, —CHO, C₁-C₆alkylcarbonyl or C₁-C₆alkoxycarbonyl substituent, and their use as insecticides, acaricides, molluscicides and nematicides.

WO 2013/079672 A1 discloses that certain substituted spiroheterocyclic pyrrolidine dione compounds, having an alkynyl-phenyl-headgroup, have herbicidal properties.

WO 2013/079708 A1 discloses cyclopentane-1,3-dione compounds and derivatives (e.g. fused and/or spirocyclic bicyclic derivatives) thereof, which are substituted at the 2-position of the cyclopentane-1,3-dione by a phenyl which itself is substituted at the 4-position by (specifically) either prop-1-ynyl or chloroethynyl and at the 2-position by (specifically) either methyl or chlorine, and derivatives of the enol ketone tautomer of such cyclopentanediones, which have herbicidal activity and/or plant-growth-inhibiting properties, especially in the control of grassy monocotyledonous weeds and/or when used post-emergence.

Heterocyclic dione compounds are now discovered, in particular pyrazolidine-dione (more particularly pyrazolidine-3,5-dione) or pyrrolidine-dione (more particularly pyrrolidine-2,4-dione) compounds or fused bicyclic derivatives of such diones, which are substituted, at the ring-carbon atom of the heterocyclic dione which is between the two oxo-substituted ring-carbons of the heterocyclic dione, by a phenyl which itself is substituted (a) at the 4-position of the phenyl by (specifically) prop-1-ynyl and (b) at a 2-position of the phenyl by (specifically) methyl or methoxy; or derivatives of the enol ketone tautomer of such heterocyclic diones. Most of the examples of these compounds which have been made and herbicidally-tested exhibit potent herbicidal activity and/or plant-growth-inhibiting properties in the control of grassy monocotyledonous weeds, e.g. after post-emergence application of the compounds (see e.g. Biological Example 1A hereinafter).

Therefore, in a first aspect of the present invention, there is provided a compound of formula (I):

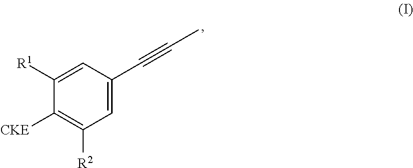

wherein CKE is of sub-formula (A) and/or (B):

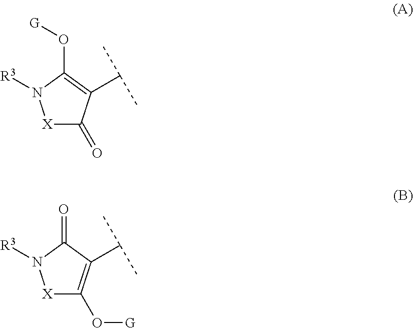

wherein:
R¹ is methyl or methoxy;
R² is hydrogen, methyl, ethyl, n-propyl, n-butyl, cyclopropyl, ethynyl, cyano, C₁-C₃alkoxy, C₁-C₂fluoroalkoxy, (difluoro)vinyloxy, C₁-C₂alkoxy-C₁-C₃alkoxy-, or C₁fluoroalkoxy-C₁-C₃alkoxy-;
X is NR⁴ or CR⁵R⁶;
wherein:
R³, R⁴, and R⁵, independently of each other, are:
hydrogen, C₁-C₄alkyl (in particular C₁-C₃alkyl, more particularly methyl or ethyl), C₁-C₂fluoroalkyl, C₁-C₃alkoxyC₁-C₃alkyl (in particular C₁-C₂alkoxyC₁-C₂alkyl such as MeOCH₂CH₂—), C₁-C₃alkylthioC₁-C₃alkyl (in particular C₁-C₂alkylthioC₁-C₂alkyl such as MeSCH₂CH₂—), C₁-C₄alkoxy (in particular methoxy), R⁹—C≡C—C(R⁷)(R⁸)—, (R¹¹)(R¹²)C═C(R¹⁰)—C(R⁷)(R⁸)—, Het-CH₂—, or Het; or
benzyl optionally substituted on its phenyl ring by 1 or 2 substituents independently being halogen (preferably fluorine, chlorine or bromine), C₁-C₂alkyl (preferably methyl), C₁fluoroalkyl (preferably trifluoromethyl), C₁-C₂alkoxy (preferably methoxy), C₁fluoroalkoxy (preferably trifluoromethoxy or difluoromethoxy), —C≡C—R¹³, —C(R¹⁴)═C(R¹⁵)(R¹⁶), —C(O)Me, —C(O)—C₁fluoroalkyl, —S(O)ₙ₄-Me, —S(O)ₙ₄—C₁fluoroalkyl, —NR¹⁹R²⁰, or cyano; or
phenyl optionally substituted by 1 or 2 substituents independently being halogen (preferably fluorine, chlorine or bromine), C₁-C₂alkyl (preferably methyl), C₁fluoroalkyl (preferably trifluoromethyl), —C≡C—R¹³, —C(R¹⁴)═C(R¹⁵)(R¹⁶), C₁-C₂alkoxy (preferably methoxy), C₁fluoroalkoxy (preferably trifluoromethoxy or difluoromethoxy), —C(O)Me, —C(O)—C₁fluoroalkyl, —S(O)ₙ₅-Me, —S(O)ₙ₅—C₁fluoroalkyl, —NR¹⁹R²⁰, or cyano;
provided that when X is NR⁴, then at least one of R³ and R⁴ (preferably R³) is hydrogen or C₁-C₃alkyl; and provided that when X is CR⁵R⁶, then at least one of R³ and R⁵ (preferably R³) is hydrogen or C₁-C₃alkyl;
   or R³ and R⁴ are taken together, and/or R³ and R⁵ are taken together, to form a chain which is (a), (b), (c), (d), (e), (f) or (g), as shown below:

—C(R²¹)(R²²)—C(R²³)(R²⁴)—Y—C(R²⁵)(R²⁶)—C(R²⁷)(R²⁸)— (a)

—C(R²¹)(R²²)—Y—C(R^{21A})(R^{22A})—C(R^{21A})(R^{22A})—C(R²⁷)(R²⁸)— (b)

—CH(R²⁹)—Y—C(R^{21A})(R^{22A})—CH(R³²)— (c)

—CH(R²⁹)—Y—CH(R³²)— (d)

—C(R²¹)(R²²)—C(R²³)(R²⁴)—C(R³⁰)(R³¹)—C(R²⁵)(R²⁶)—C(R²⁷)(R²⁸)— (e)

—C(R²¹)(R²²)—C(R^{21A})(R^{22A})—C(R³⁰)(R³¹)—C(R²⁷)(R²⁸)— (f)

—CH(R²⁹)—C(R³⁰)(R³¹)—CH(R³²)— (g)

wherein Y is O or NR⁴⁰;
and R⁶ is hydrogen or C₁-C₃alkyl;
or R⁵ and R⁶ taken together are —(CH₂)ₙ₁— or —(CH₂)ₙ₂—X¹—(CH₂)ₙ₃—, in which case R³ is as defined herein (preferably hereinabove) provided that R³ and R⁵ are not taken together;
wherein X¹ is O, C(H)(C₁-C₂alkyl), C(C₁-C₂alkyl)₂ or C(H)(C₁-C₂alkoxy);
n1 is 2, 3, 4 or 5 (preferably n1 is 4 or 5); and
n2 and n3 are independently 1, 2 or 3 provided that n2+n3 is 2, 3 or 4 (preferably n2+n3 is 3 or 4);
and wherein:
Het is a heteroaryl, attached at a ring-carbon, which is optionally substituted by 1, 2 or 3 (preferably 1 or 2, more preferably 1) ring-carbon substituents independently being halogen (preferably fluorine or chlorine), C₁-C₃alkyl (preferably C₁-C₂alkyl), C₁-C₂fluoroalkyl (preferably C₁fluoroalkyl), —C≡C—R¹³ (preferably ethynyl or prop-1-ynyl), —C(R¹⁴)═C(R¹⁵)(R¹⁶) (preferably ethenyl or prop-1-enyl), C₁-C₃alkoxy (preferably C₁-C₂alkoxy such as methoxy), C₁-C₂fluoroalkoxy (preferably C₁fluoroalkoxy), cyclopropyloxy, CH₂═CH—CH₂—O—, HC≡C—CH₂—O—, —C(O)—R¹⁷, —S(O)ₙ₁₈—R¹⁸, —NR¹⁹R²⁰, cyano or nitro; provided that any non-fluorine halogen, alkoxy, fluoroalkoxy, cyclopropyloxy, CH₂═CH—CH₂—O— or HC≡C—CH₂—O— is not substituted at any ring-carbon bonded directly to a ring-nitrogen of the heteroaryl;
and/or, when Het is a 5-membered heteroaryl ring containing a ring-nitrogen atom not partaking in a C═N ring double bond, the heteroaryl is optionally substituted on the ring-nitrogen atom not partaking in a C═N ring double bond by one C₁-C₃alkyl, C₁-C₂fluoroalkyl, C₁-C₃alkyl-C(O)—, C₁-C₂fluoroalkyl-C(O)— or C₁-C₂alkyl-S(O)₂— substituent;
and wherein:
R⁷ and R⁸, independently of each other, are hydrogen or methyl;
R⁹ is hydrogen, methyl, ethyl or chlorine; and
R¹⁰, R¹¹ and R¹², independently of each other, are hydrogen, methyl or ethyl, provided that R¹⁰, R¹¹ and R¹² in total contain no more than three carbon atoms;
R¹³ is hydrogen, methyl, C₁fluoroalkyl (in particular trifluoromethyl), fluorine or chlorine;
R¹⁴, R¹⁵ and R¹⁶ independently are hydrogen, methyl, C₁fluoroalkyl (in particular trifluoromethyl), fluorine or chlorine; provided that R¹⁴, R¹⁵ and R¹⁶ in total contain no more than one carbon atom, and R¹⁴, R¹⁵ and R¹⁶ in total comprise no more than one chlorine; and
n4 is 0, 1 or 2;
n5 is 0, 1 or 2;
R¹⁷ and R¹⁸ independently are C₁-C₃alkyl, C₁fluoroalkyl, or —NR^{19A}R^{20A};
n18 is 0, 1 or 2;
R¹⁹ is —C(O)—C₁-C₂alkyl, —C(O)—C₁fluoroalkyl, —S(O)₂—C₁-C₂alkyl, —S(O)₂—C₁fluoroalkyl, C₁-C₂alkyl, or C₁fluoroalkyl;
R²⁰ and R^{20A} independently are hydrogen, methyl or C₁fluoroalkyl; and
R^{19A} is hydrogen, C₁-C₂alkyl or C₁fluoroalkyl;
and wherein:
R²¹, R²², R²⁷ and R²⁸ are independently hydrogen or methyl; provided that two, three or all of R²¹, R²², R²⁷ and R²⁸ are hydrogen; and
R^{21A} and R^{22A} are independently hydrogen or methyl; and
R²³, R²⁴, R²⁵, and R²⁶ are independently hydrogen, methyl, ethyl or methoxymethyl; provided that two, three or all of R²³, R²⁴, R²⁵ and R²⁶ are hydrogen;
or R²³ and R²⁵ are taken together and form a —CH₂— or —CH₂CH₂— bridge, and R²⁴ and R²⁶ are hydrogen; and
R²⁹ and R³² are independently hydrogen or methyl; and
R³⁰ is hydrogen, C₁-C₃alkyl, C₁-C₂fluoroalkyl, C₁-C₃alkoxy (preferably methoxy), C₁-C₃fluoroalkoxy (in particular CF₃CH₂O—), C₁-C₃alkyl-S(O)ₙ₃₀—, C₁-C₂alkoxyC₁-C₂alkyl (in particular methoxymethyl), or C₁-C₂alkylthioC₁-C₂alkyl (preferably R³⁰ is hydrogen or methoxy, more preferably hydrogen);
wherein n30 is 0, 1 or 2 (preferably 0); and R³¹ is hydrogen, methyl or ethyl (preferably R³¹ is hydrogen);
or R³⁰ and R³¹ taken together are oxo (═O), ═N—O—H, or ═N—O—(C₁-C₃alkyl);
provided that, in all cases wherein R³ and R⁴ are taken together, and/or R³ and R⁵ are taken together, to form a chain which is (a), (b), (c), (d), (e), (f) or (g), then the chain contains a total of no more than three substituents (preferably a total of none, one or two substituents) on carbon in the ring which is formed by NR³, X, and the chain (a), (b), (c), (d), (e), (f) or (g); wherein if a bridge is formed by R²³ and R²⁵ being taken together then this bridge is counted as two ring-carbon-substituents;
and wherein R⁴⁰ is C₁-C₃alkoxy, C₁-C₂fluoroalkoxy (in particular C₁fluoroalkoxy or CF₃CH₂—), cyclopropyloxy, CH₂═CH—CH₂—O—, HC≡C—CH₂—O—, N≡C—CH₂O—, CH₃OCH₂O—, CH₃OCH₂CH₂O—, CH₃CH₂OCH₂O—, C₁-C₃alkyl or C₁fluoroalkyl;
and wherein:
G is hydrogen; an agriculturally acceptable metal, or an agriculturally acceptable sulfonium or ammonium group; or
G is —C(X^{a})—R^{a}, —C(X^{b})—X^{c}—R^{b}, —C(X^{d})—N(R^{c})—R^{d}, —SO₂—R^{e}, —P(X^{e})(R^{f})—R^{g}, —CH₂—X^{f}—R^{h}; or phenyl-CH₂— or phenyl-CH(C₁-C₂alkyl)- (in each of which the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or heteroaryl-CH₂— or heteroaryl-CH(C₁-C₂alkyl)- (in each of which the heteroaryl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or phenyl-C(O)—CH₂— (wherein the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro); or C₁-C₆alkoxy-C(O)—CH₂—, C₁-C₆alkoxy-C(O)—CH═CH—, C₂-C₇alken-1-yl-CH₂—, C₂-C₇alken-1-yl-CH(C₁-C₂alkyl)-, C₂-C₄fluoroalken-1-yl-CH₂—, C₂-C₇alkyn-1-yl-CH₂—, or C₂-C₇alkyn-1-yl-CH(C₁-C₂alkyl)-;
wherein X^{a}, X^{b}, X^{c}, X^{d}, X^{e} and X^{f} are independently of each other oxygen or sulfur (preferably oxygen); and wherein
R^{a} is H, C₁-C₂₁alkyl, C₂-C₂₁alkenyl, C₂-C₁₈alkynyl, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro;
R^{b} is C₁-C₁₈alkyl, C₃-C₁₈alkenyl, C₃-C₁₈alkynyl, C₂-C₁₀fluoroalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroarylC₁-C₅alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkyl-thio, C₁-C₃alkylsulfinyl, C₁-C₃alkylsulfonyl, halogen, cyano, or nitro), C₃-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; and
R^{c} and R^{d} are each independently of each other hydrogen, C₁-C₁₀alkyl, C₃-C₁₀alkenyl, C₃-C₁₀alkynyl, C₂-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; diheteroarylamino or diheteroarylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; phenylamino or phenylamino substituted by 1, 2 or 3 of, independently, C₁-C₃alkyl, C₁-C₃fluoroalkyl, C₁-C₃alkoxy, C₁-C₃fluoroalkoxy, halogen, cyano or by nitro; diphenylamino or diphenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or C₃-C₇cycloalkylamino, di(C₃-C₇cycloalkyl)amino or C₃-C₇cycloalkoxy;
or R^{c} and R^{d}, together with the nitrogen to which they are bonded, to form an unsubstituted 4, 5, 6 or 7 (e.g. 5 or 6) membered ring, optionally containing one heteroatom selected from O or S; and
R^{e} is C₁-C₁₀alkyl, C₂-C₁₀alkenyl, C₂-C₁₀alkynyl, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; diheteroarylamino or diheteroarylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; phenylamino or phenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; diphenylamino or diphenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or C₃-C₇cycloalkylamino, di(C₃-C₇cycloalkyl)amino, C₃-C₇cycloalkoxy, C₁-C₁₀alkoxy, C₁-C₁₀fluoroalkoxy, C₁-C₅alkylamino or di(C₁-C₄alkyl)amino;
R^{f} and R^{g} are each independently of each other C₁-C₁₀alkyl, C₂-C₁₀alkenyl, C₂-C₁₀alkynyl, C₁-C₁₀alkoxy, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; diheteroarylamino or diheteroarylamino substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; phenylamino or phenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; diphenylamino or diphenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or C₃-C₇cycloalkylamino, di(C₃-C₇cycloalkyl)amino, C₃-C₇cycloalkoxy, C₁-C₁₀fluoroalkoxy, C₁-C₅alkylamino or di(C₁-C₄alkyl)amino; or benzyloxy or phenoxy, wherein the benzyl and phenyl groups are in turn optionally substituted by 1, 2 or 3 of, independently, C₁-C₃alkyl, C₁-C₃fluoroalkyl, C₁-C₃alkoxy, C₁-C₃fluoroalkoxy, halogen, cyano or nitro; and
R^{h} is C₁-C₁₀alkyl, C₃-C₁₀alkenyl, C₃-C₁₀alkynyl, C₁-C₁₀fluoroalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), phenoxy(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), heteroaryloxy(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), C₃-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; C₁-C₆alkyl-C(O)—; or phenyl-C(O)— wherein the phenyl is optionally substituted by 1 or 2 of, independently, C₁-C₂alkyl, C₁fluoroalkyl, C₁-C₂alkoxy, C₁fluoroalkoxy, fluorine, chlorine, bromine, cyano or nitro;
wherein “heteroaryl” means an aromatic ring system containing at least one ring heteroatom and consisting either of a single ring or of two fused rings;
and wherein the compound of formula (I) is optionally present (e.g. where chemically possible) as an agrochemically acceptable salt thereof.

In the substituent definitions of the compounds of the formula I, each alkyl moiety either alone or as part of a larger group (such as alkoxy, alkylthio, alkoxycarbonyl, alkylcarbonyl, alkylaminocarbonyl, or dialkylaminocarbonyl, et al.) can be straight-chained or branched. Typically, the alkyl is, for example, methyl, ethyl, n-propyl, isopropyl, n-butyl, sec-butyl, isobutyl, tert-butyl, n-pentyl, neopentyl, or n-hexyl. The alkyl groups can e.g. be C₁-C₆alkyl groups (except where already defined more narrowly), but are preferably C₁-C₄alkyl or C₁-C₃alkyl groups (except where already defined more narrowly), and, more preferably, are C₁-C₂alkyl groups such as methyl.

Alkenyl and alkynyl moieties can be in the form of straight or branched chains, and the alkenyl moieties, where appropriate, can be of either the (E)- or (Z)-configuration. The alkenyl or alkynyl are typically C₂-C₃alkenyl or C₂-C₃alkynyl such as vinyl, allyl, ethynyl, propargyl or prop-1-ynyl. Alkenyl and alkynyl moieties can contain one or more double and/or triple bonds in any combination; but preferably contain only one double bond (for alkenyl) or only one triple bond (for alkynyl).

Halogen is fluorine, chlorine, bromine or iodine. Preferred halogens are fluorine, chlorine or bromine.

Fluoroalkyl groups are alkyl groups which are substituted with one or more (e.g. 1, 2, 3, 4 or 5; in particular 1, 2 or 3; e.g. 1 or 2) fluorine atoms. Fluoroalkyl is typically C₁-C₃fluoroalkyl or C₁-C₂fluoroalkyl (preferably C₁fluoroalkyl), such as CF₃, CHF₂, CH₂F, CH₃CHF—, CF₃CH₂—, CHF₂CH₂—, CH₂FCH₂—, CHF₂CF₂— or (CH₃)₂CF—. Fluoroalkoxy is typically C₁-C₃fluoroalkoxy or C₁-C₂fluoroalkoxy (preferably C₁fluoroalkoxy), such as CF₃O, CHF₂O, CH₂FO, CH₃CHFO—, CF₃CH₂O—, CHF₂CH₂O— or CH₂FCH₂O—.

In the context of the present specification the term “aryl” means phenyl or naphthyl. A preferred aryl group is phenyl.

The term “heteroaryl” as used herein means an aromatic ring system containing at least one ring heteroatom and consisting either of a single ring or of two fused rings. Preferably, single rings will contain 1, 2 or 3 ring heteroatoms and bicyclic systems 1, 2, 3 or 4 ring heteroatoms which will preferably be selected from nitrogen, oxygen and sulfur. Typically, a “heteroaryl” is furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl or indolizinyl; optionally present, where chemically possible, as an agrochemically acceptable salt thereof.

The term “heterocyclyl” as used herein, except where explicitly stated otherwise, means a 4, 5, 6 or 7 (in particular 5, 6 or 7) membered monocyclic organic ring or a 8, 9, 10 or 11 (in particular 8, 9 or 10) membered fused bicyclic organic ring system, which is fully saturated, and which has one or two (preferably one) ring heteroatoms independently selected from oxygen, sulfur and nitrogen. Where the heterocyclyl has two ring heteroatoms, preferably, the two ring heteroatoms are separated by at least two ring carbon atoms. Preferably, the heterocyclyl is attached at a ring carbon atom within the heterocyclyl. In particular, the heterocyclyl can be tetrahydrofuranyl, tetrahydropyranyl, tetrahydrothiophenyl, 1,4-dioxanyl, 1,4-dithianyl, morpholinyl, thiomorpholinyl, pyrrolidinyl, piperidinyl or piperazinyl; more particularly tetrahydrofuranyl (e.g. tetrahydrofuran-2-yl or particularly tetrahydrofuran-3-yl), tetrahydropyranyl (e.g. tetrahydropyran-2-yl, tetrahydropyran-3-yl or particularly tetrahydropyran-4-yl), morpholinyl, pyrrolidinyl (e.g. pyrrolidin-2-yl or particularly pyrrolidin-3-yl), piperidinyl (e.g. piperidin-2-yl, piperidin-3-yl or particularly piperidin-4-yl) or piperazinyl. In a particular embodiment, the heterocyclyl, when optionally substituted, is optionally substituted by 1 or 2 (e.g. 1) ring-carbon substituents independently being C₁-C₃alkyl (e.g. C₁-C₂alkyl), C₁-C₂fluoroalkyl or oxo (═O), and/or is optionally substituted by one C₁-C₃alkyl (e.g. C₁-C₂alkyl), C₁-C₂fluoroalkyl or C₁-C₃alkoxy (e.g. C₁-C₂alkyl or C₁-C₂fluoroalkyl) substituent on a ring nitrogen if present, and/or is optionally substituted by one or two oxo (═O) substituents on a ring sulfur if present.

Preferably, a cycloalkyl is cyclopropyl, cyclobutyl, cyclopentyl or cyclohexyl. (Cycloalkyl)alkyl is preferably (cycloalkyl)methyl such as (C₃-C₆cycloalkyl)methyl in particular cyclopropylmethyl. Preferably, cycloalkenyl is cyclopentenyl or cyclohexenyl.

The invention relates also to the agriculturally acceptable salts which the compounds of formula I are able to form with transition metal, alkali metal and alkaline earth metal bases, amines, quaternary ammonium bases or tertiary sulfonium bases.

Among the transition metal, alkali metal and alkaline earth metal salt formers, special mention should be made of the hydroxides of copper, iron, lithium, sodium, potassium, magnesium and calcium, and preferably the hydroxides, bicarbonates and carbonates of sodium and potassium.

Examples of amines suitable for ammonium salt formation include ammonia as well as primary, secondary and tertiary C₁-C₁₈alkylamines, C₁-C₄hydroxyalkylamines and C₂-C₄alkoxyalkyl-amines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, di-isopropylamine, di-n-butylamine, di-n-amylamine, di-isoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, trimethylamine, triethylamine, tri-n-propylamine, tri-isopropylamine, tri-n-butylamine, tri-isobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and di-isopropylamine.

Preferred quaternary ammonium bases suitable for salt formation correspond, for example, to the formula [N(RₐR_{b}R_{c}R_{d})]OH, wherein Rₐ, R_{b}, R_{c} and R_{d} are each independently of the others hydrogen, C₁-C₄alkyl. Further suitable tetraalkylammonium bases with other anions can be obtained, for example, by anion exchange reactions.

Preferred tertiary sulfonium bases suitable for salt formation correspond, for example, to the formula [SRₑR_{f}R_{g}]OH, wherein Rₑ, R_{f} and R_{g} are each independently of the others C₁-C₄ alkyl. Trimethylsulfonium hydroxide is especially preferred. Suitable sulfonium bases may be obtained from the reaction of thioethers, in particular dialkylsulfides, with alkylhalides, followed by conversion to a suitable base, for example a hydroxide, by anion exchange reactions.

It should be understood that in those compounds of formula I, where G is a metal, ammonium or sulfonium as mentioned above and as such represents a cation, the corresponding negative charge is largely delocalised across the O—C═C—C═O unit.

The compounds of formula I according to the invention also include hydrates which may be formed during the salt formation.

The latentiating groups (i.e. leaving or removable groups) within G (for example, without limitation, the latentiating groups where G is —C(X^{a})—R^{a} or —C(X^{b})—X^{c}—R^{b}, et al.) are generally selected to allow their removal, typically by one or a combination of biochemical, chemical or physical processes, to afford the corresponding compound of formula (I) where G is H, before, during or following (preferably during or following) application of the compound of formula (I) to the treated area (e.g. field) or to plants. Examples of these processes include enzymatic cleavage or other in/on-plant cleavage (e.g. cleavage of ester and/or carbonate moieties), chemical hydrolysis, and/or photoloysis. Some compounds bearing such groups G occasionally offer certain advantages or different technical properties, such as improved and/or more consistent and/or different penetration of the cuticula of the plants treated, increased and/or different tolerance of certain crops, improved and/or different compatibility or stability in formulated mixtures containing other herbicides, herbicide safeners, plant growth regulators, fungicides or insecticides, or reduced and/or different leaching properties in soils.

The preferred (including more or most preferred et al.), particular, suitable, alternative and/or optional values of the substituents in, or other features of, the compound of formula (I), in particular G, X, X¹, Y, R¹, R², R^{2A}, R^{2B}, R^{2C}, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R^{21A}, R²², R^{22A}, R²³, R²⁴, R²⁵, R²⁶, R²⁷, R²⁸, R²⁹, R³⁰, R³¹, R³², R³³, R⁴⁰, R^{a}, R^{b}, R^{c}, R^{d}, R^{e}, R^{f}, R^{g}, R^{h}, X^{a}, X^{b}, X^{c}, X^{d}, X^{e}, X^{f}, Het, n1, n2, n3, n4, n5, n18, and/or n30, and/or all other features of or within the compound of formula (I), are set out below (and/or in some cases generally herein e.g. hereinabove), and can be either taken alone or taken together with one or more of any other preferred, particular, suitable, alternative and/or optional features in any combination(s) thereof.

When X is NR⁴ and CKE is of sub-formula (A), which is the generally preferred option when X is NR⁴, then the compound of formula (I) is a compound of formula (I-A1):

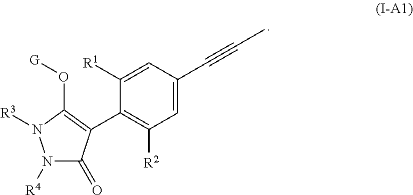

When X is NR⁴ and CKE is of sub-formula (B), then the compound of formula (I) is a compound of formula (I-B1):

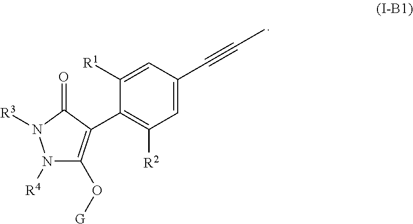

When X is CR⁵R⁶ and CKE is of sub-formula (A), then the compound of formula (I) is a compound of formula (I-A2):

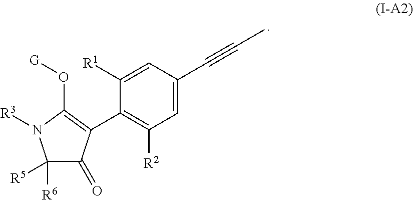

When X is CR⁵R⁶ and CKE is of sub-formula (B), which is the preferred option when X is CR⁵R⁶, then the compound of formula (I) is a compound of formula (I-B2):

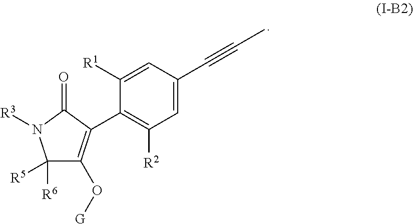

In the compounds of the invention (e.g. in the compounds of formula (I), in one preferred embodiment, G is hydrogen; an agriculturally acceptable metal (e.g. an agriculturally acceptable alkali metal or alkaline earth metal), or an agriculturally acceptable sulfonium or ammonium group; or G is —C(X^{a})—R^{a} or —C(X^{b})—X^{c}—R^{b}, wherein X^{a}, R^{a}, X^{b}, X^{c} and R^{b} are as defined herein.

In a particular embodiment, G is a group —C(X^{a})—R^{a} or —C(X^{b})—X^{c}—R^{b}, wherein X^{a}, R^{a}, X^{b}, X^{c} and R^{b} are as defined herein.

Preferably, X^{a}, X^{b}, X^{c}, X^{d}, X^{e} and/or X^{f} are oxygen. More preferably, X^{a}, X^{b}, X^{c}, X^{d}, X^{e} and X^{f} are oxygen.

Preferably, R^{a} is C₁-C₁₀alkyl (e.g. C₁-C₆alkyl), C₂-C₆alkenyl (e.g. C₂-C₄alkenyl), C₂-C₆alkynyl (e.g. C₂-C₄alkynyl), C₃-C₆cycloalkyl or C₁-C₄alkoxyC₁-C₄alkyl.

Preferably, R^{b} is C₁-C₁₀alkyl (e.g. C₁-C₆alkyl), C₂-C₅alkenyl-CH₂— (e.g. C₂-C₃alkenyl-CH₂—), C₂-C₄alkenyl-CH(Me)- (e.g. C₂-C₃alkenyl-CH(Me)-), C₂-C₅alkynyl-CH₂— (e.g. C₂-C₃alkynyl-CH₂—), C₂-C₄alkynyl-CH(Me)- (e.g. C₂-C₃alkynyl-CH(Me)-), C₃-C₆cycloalkyl or C₁-C₄alkoxyC₁-C₄alkyl.

When G is —C(X^{a})—R^{a} or —C(X^{b})—X^{c}—R^{b}, then preferably X^{a}, X^{b} and X^{c} are oxygen, R^{a} is C₁-C₁₀alkyl (e.g. C₁-C₆alkyl), C₂-C₆alkenyl (e.g. C₂-C₄alkenyl), C₂-C₆alkynyl (e.g. C₂-C₄alkynyl), C₃-C₆cycloalkyl or C₁-C₄alkoxyC₁-C₄alkyl; and R^{b} is C₁-C₁₀alkyl (e.g. C₁-C₆alkyl), C₂-C₅alkenyl-CH₂— (e.g. C₂-C₃alkenyl-CH₂—), C₂-C₄alkenyl-CH(Me)- (e.g. C₂-C₃alkenyl-CH(Me)-), C₂-C₅alkynyl-CH₂— (e.g. C₂-C₃alkynyl-CH₂—), C₂-C₄alkynyl-CH(Me)- (e.g. C₂-C₃alkynyl-CH(Me)-), C₃-C₆cycloalkyl or C₁-C₄alkoxyC₁-C₄alkyl.

In a preferable embodiment, G is hydrogen, or an agriculturally acceptable alkali metal or alkaline earth metal, or an agriculturally acceptable sulfonium or ammonium group. More preferably, G is hydrogen, or an agriculturally acceptable alkali metal or alkaline earth metal.

In a preferable embodiment, G is hydrogen, —C(X^{a})—R^{a} or —C(X^{b})—X^{c}—R^{b}.

Most preferably G is hydrogen.

In one preferred embodiment of the invention, R¹ is methyl.

In another preferred embodiment of the invention, R¹ is methoxy.

In the invention, R² is hydrogen, methyl, ethyl, n-propyl, n-butyl, cyclopropyl, ethynyl, cyano, C₁-C₃alkoxy (e.g. methoxy, ethoxy, n-propoxy or isopropoxy), C₁-C₂fluoroalkoxy (e.g. C₂fluoroalkoxy such as C₁fluoroalkylmethoxy e.g. CF₃CH₂O—, or C₁fluoroalkoxy such as difluoromethoxy or trifluoromethoxy), (difluoro)vinyloxy (i.e. CF₂═CH—O—), C₁-C₂alkoxy-C₁-C₃alkoxy-, or C₁fluoroalkoxy-C₁-C₃alkoxy-.

Preferably, when R² is C₁-C₂alkoxy-C₁-C₃alkoxy- or C₁fluoroalkoxy-C₁-C₃alkoxy-, then R² is R^{2A}O—CH(R^{2B})—CH(R^{2C})—O—;

wherein R^{2A} is C₁-C₂alkyl (in particular methyl) or C₁fluoroalkyl; and
R^{2B} and R^{2C} are independently hydrogen or methyl, provided that one or both of R^{2B} and R^{2C} are hydrogen.

Preferably, R^{2A} is methyl or C₁fluoroalkyl, more preferably methyl.

Preferably, both of R^{2B} and R^{2C} are hydrogen.

More preferably, when R² is C₁-C₂alkoxy-C₁-C₃alkoxy- or C₁fluoroalkoxy-C₁-C₃alkoxy- (in particular when R² is R^{2A}O—CH(R^{2B})—CH(R^{2C})—O—), then R² is MeO—CH₂—CH₂—O—.

Preferably, e.g. in all aspects and/or embodiments of the invention, R² is hydrogen, methyl, ethyl, n-propyl, n-butyl, cyclopropyl, ethynyl, cyano, methoxy, ethoxy, C₁fluoroalkoxy, C₁fluoroalkylmethoxy (in particular CF₃CH₂O—), (difluoro)vinyloxy (i.e. CF₂═CH—O—), or MeO—CH₂—CH₂—O—.

Alternatively or additionally, preferably, e.g. in all aspects and/or embodiments of the invention, R² is hydrogen, methyl, ethyl, ethynyl, methoxy, ethoxy, C₁fluoroalkoxy (e.g. monofluoromethoxy, difluoromethoxy or trifluoromethoxy), C₁fluoroalkylmethoxy (in particular CF₃CH₂O—), or MeO—CH₂—CH₂—O—.

Preferably, e.g. in all aspects and/or embodiments of the invention, when R¹ is methyl then R² is not hydrogen.

More preferably, e.g. in all aspects and/or embodiments of the invention:

R¹ is methyl or methoxy, and R² is methyl, ethyl, ethynyl, methoxy, ethoxy, C₁fluoroalkoxy (e.g. monofluoromethoxy, difluoromethoxy or trifluoromethoxy), C₁fluoroalkylmethoxy (in particular CF₃CH₂O—), or MeO—CH₂—CH₂—O—;
or R¹ is methoxy and R² is hydrogen.

Even more preferably, e.g. in all aspects and/or embodiments of the invention:

R¹ is methyl or methoxy, and R² is methyl, ethyl, methoxy or ethoxy;
or R¹ is methoxy and R² is hydrogen.

Still more preferably, e.g. in all aspects and/or embodiments of the invention:

R¹ is methyl or methoxy, and R² is methyl, ethyl or methoxy;
or R¹ is methoxy and R² is hydrogen.

Most preferably, e.g. in all aspects and/or embodiments of the invention, R¹ is methyl and R² is methyl; or R¹ is methoxy and R² is hydrogen or methoxy.

Preferably, e.g. in all aspects and/or embodiments of the invention,

R¹ is methyl, and
R² is hydrogen, methyl, ethyl, ethynyl, ethoxy, C₁fluoroalkoxy (e.g. monofluoromethoxy, difluoromethoxy or trifluoromethoxy), C₁fluoroalkylmethoxy (in particular CF₃CH₂O—), or MeO—CH₂—CH₂—O—.

More preferably, e.g. in all aspects and/or embodiments of the invention:

R¹ is methyl, and
R² is methyl, ethyl, ethynyl, ethoxy, C₁fluoroalkoxy (e.g. monofluoromethoxy, difluoromethoxy or trifluoromethoxy), C₁fluoroalkylmethoxy (in particular CF₃CH₂O—), or MeO—CH₂—CH₂—O—; even more preferably methyl, ethyl or ethoxy; most preferably methyl.

Most preferably e.g. in all aspects and/or embodiments of the invention:

R¹ is methoxy, and
R² is hydrogen, methyl, ethyl, ethynyl, methoxy, ethoxy, C₁fluoroalkoxy (e.g. monofluoromethoxy, difluoromethoxy or trifluoromethoxy), C₁fluoroalkylmethoxy (in particular CF₃CH₂O—), or MeO—CH₂—CH₂—O—; even more preferably hydrogen, methyl, ethyl, methoxy or ethoxy; most preferably hydrogen or methoxy.

Particularly preferably, e.g. in all aspects and/or embodiments of the invention, X is NR⁴.

It is particularly preferable, e.g. in all aspects and/or embodiments of the invention, that R⁵ and R⁶ are not taken together to be —(CH₂)ₙ₁— or —(CH₂)ₙ₂—X¹—(CH₂)ₙ₃—. Therefore, preferably R⁶ is hydrogen or methyl. Most preferably, R⁶ is hydrogen.

Preferably, e.g. in all aspects and/or embodiments of the invention, R³ is hydrogen, C₁-C₃alkyl, C₁-C₂fluoroalkyl (in particular C₁fluoroalkyl), or Het-CH₂—; or R³ and R⁴ are taken together, and/or R³ and R⁵ are taken together, to form a chain which is (a), (b), (c), (d), (e), (f) or (g), as defined herein.

More preferably, R³ is hydrogen, C₁-C₂fluoroalkyl (in particular C₁fluoroalkyl), or C₁-C₃alkyl; or R³ and R⁴ are taken together, and/or R³ and R⁵ are taken together, to form a chain which is (a), (b), (c), (d), (e), (f) or (g), as defined herein.

Preferably, e.g. in all aspects and/or embodiments of the invention, R⁴ and/or R⁵, independently of each other, is or are:
hydrogen, C₁-C₃alkyl (in particular methyl or ethyl), C₁-C₂fluoroalkyl (in particular C₁fluoroalkyl), C₁-C₂alkoxyC₁-C₂alkyl (in particular MeOCH₂CH₂—), C₁-C₂alkylthioC₁-C₂alkyl (in particular MeSCH₂CH₂—), C₁-C₂alkoxy (in particular methoxy), R⁹—C≡C—C(R⁷)(R⁸)—, or Het-CH₂—; or
benzyl optionally substituted on its phenyl ring by 1 or 2 substituents independently being fluorine, chlorine, bromine, C₁-C₂alkyl (preferably methyl), C₁fluoroalkyl (preferably trifluoromethyl), C₁-C₂alkoxy (preferably methoxy), C₁fluoroalkoxy (preferably trifluoromethoxy or difluoromethoxy), —C≡C—R¹³, —C(R¹⁴)═C(R¹⁵)(R¹⁶), —C(O)Me, —C(O)—C₁fluoroalkyl, —S(O)ₙ₄-Me, —S(O)ₙ₄—C₁fluoroalkyl, —NR¹⁹R²⁰, or cyano;
provided that when X is NR⁴, then at least one of R³ and R⁴ (preferably R³) is hydrogen or C₁-C₃alkyl; and provided that when X is CR⁵R⁶, then at least one of R³ and R⁵ (preferably R³) is hydrogen or C₁-C₃alkyl;
   or R³ and R⁴ are taken together, and/or R³ and R⁵ are taken together, to form a chain which is (a), (b), (c), (d), (e), (f) or (g), as defined herein.

More preferably, e.g. in all aspects and/or embodiments of the invention, R⁴ and/or R⁵, independently of each other, is or are:
hydrogen, C₁-C₃alkyl (in particular methyl or ethyl), C₁-C₂fluoroalkyl (in particular C₁fluoroalkyl), MeOCH₂CH₂—, MeSCH₂CH₂—, C₁-C₂alkoxy (in particular methoxy), R⁹—C≡C—C(R⁷)(R⁸)—, or Het-CH₂—; or
benzyl optionally substituted on its phenyl ring by 1 or 2 substituents independently being fluorine, chlorine, methyl, C₁fluoroalkyl (preferably trifluoromethyl), methoxy, C₁fluoroalkoxy (preferably trifluoromethoxy or difluoromethoxy), —C≡C—R¹³, or cyano;
provided that when X is NR⁴, then at least one of R³ and R⁴ (preferably R³) is hydrogen or C₁-C₃alkyl; and provided that when X is CR⁵R⁶, then at least one of R³ and R⁵ (preferably R³) is hydrogen or C₁-C₃alkyl;
   or R³ and R⁴ are taken together, and/or R³ and R⁵ are taken together, to form a chain which is (a), (b), (c), (d), (e), (f) or (g), as defined herein.

Even more preferably, e.g. in all aspects and/or embodiments of the invention, R⁴ and/or R⁵, independently of each other, is or are: hydrogen, C₁-C₃alkyl (in particular methyl or ethyl), R⁹—C≡C—C(R⁷)(R⁸)—, or Het-CH₂—;
provided that when X is NR⁴, then at least one of R³ and R⁴ (preferably R³) is hydrogen or C₁-C₃alkyl; and provided that when X is CR⁵R⁶, then at least one of R³ and R⁵ (preferably R³) is hydrogen or C₁-C₃alkyl;
   or R³ and R⁴ are taken together, and/or R³ and R⁵ are taken together, to form a chain which is (a), (b), (c), (d), (e), (f) or (g), as defined herein.

Preferably, e.g. in all aspects and/or embodiments of the invention, R³ is not hydrogen; and/or, preferably, R⁴ and/or R⁵ is or are not hydrogen.

In one particularly preferred embodiment of the invention:

R³ is C₁-C₃alkyl (preferably methyl or ethyl) or C₁-C₂fluoroalkyl (in particular C₁fluoroalkyl); and/or
R⁴ and/or R⁵ is or are, independently, C₁-C₃alkyl (preferably methyl or ethyl) or C₁-C₂fluoroalkyl (in particular C₁fluoroalkyl).

Preferably, e.g. in all aspects and/or embodiments of the invention, Het is a heteroaryl (in particular monocyclic heteroaryl), attached at a ring-carbon, which is optionally substituted by 1, 2 or 3 (in particular 1 or 2, e.g. 1) ring-carbon substituents independently being C₁-C₂alkyl, C₁fluoroalkyl, C₁-C₂alkyl-C(O)—, C₁fluoroalkyl-C(O)—, ethynyl, prop-1-ynyl, C₁-C₂alkoxy, C₁fluoroalkoxy, fluorine, chlorine, bromine, cyano or nitro, provided that any chlorine, bromine, alkoxy or fluoroalkoxy is not substituted at any ring-carbon bonded directly to a ring-nitrogen of the heteroaryl;

and/or, in the case of a 5-membered heteroaryl ring containing a ring-nitrogen atom not partaking in a C═N ring double bond, the heteroaryl is optionally substituted on the ring-nitrogen atom not partaking in a C═N ring double bond by one C₁-C₃alkyl, C₁-C₂fluoroalkyl, C₁-C₃alkyl-C(O)—, C₁-C₂fluoroalkyl-C(O)— or C₁-C₂alkyl-S(O)₂— substituent.

More preferably, e.g. in all aspects and/or embodiments of the invention, Het is a heteroaryl (in particular monocyclic heteroaryl), attached at a ring-carbon, which is optionally substituted by 1 or 2 (in particular 1) ring-carbon substituents independently being C₁-C₂alkyl (in particular methyl), C₁fluoroalkyl (in particular CF₃), C₁-C₂alkyl-C(O)— (in particular Me-C(O)—), C₁fluoroalkyl-C(O)—, ethynyl, prop-1-ynyl, fluorine or cyano;

and/or, in the case of a 5-membered heteroaryl ring containing a ring-nitrogen atom not partaking in a C═N ring double bond, the heteroaryl is optionally substituted on the ring-nitrogen atom not partaking in a C═N ring double bond by one C₁-C₂alkyl (e.g. methyl), C₁fluoroalkyl, methyl-C(O)— or C₁fluoroalkyl-C(O)— substituent.

More preferably, e.g. in all aspects and/or embodiments of the invention, Het is a heteroaryl (in particular monocyclic heteroaryl), attached at a ring-carbon, which is optionally substituted by 1 or 2 (in particular 1) ring-carbon substituents independently being C₁-C₂alkyl (in particular methyl), C₁fluoroalkyl (in particular CF₃), fluorine or cyano;

and/or, in the case of a 5-membered heteroaryl ring containing a ring-nitrogen atom not partaking in a C═N ring double bond, the heteroaryl is optionally substituted on the ring-nitrogen atom not partaking in a C═N ring double bond by one methyl substituent.

Preferably, e.g. in all aspects and/or embodiments of the invention, Het is an optionally substituted monocyclic heteroaryl, attached at a ring-carbon. Such as monocyclic heteroaryl can be 5-membered or 6-membered monocyclic heteroaryl.

More preferably, e.g. in all aspects and/or embodiments of the invention, Het is an optionally substituted monocyclic heteroaryl, attached at a ring-carbon, which is:

pyridinyl (preferably pyridin-3-yl or most preferably pyridin-2-yl), pyrazolyl (preferably pyrazol-5-yl or pyrazol-4-yl, or most preferably pyrazol-3-yl), imidazolyl (preferably imidazol-2-yl), pyrazinyl, pyrimidinyl (preferably pyrimidin-4-yl), pyridazinyl (preferably pyridazin-3-yl), triazolyl (e.g. 1,2,3-triazolyl), tetrazol-5-yl, oxazolyl, thiazolyl, isoxazolyl, isothiazolyl or oxadiazolyl.

Even more preferably, e.g. in all aspects and/or embodiments of the invention, Het is an optionally substituted monocyclic heteroaryl, attached at a ring-carbon, which is:

pyridinyl (preferably pyridin-3-yl or most preferably pyridin-2-yl), pyrazolyl (preferably pyrazol-5-yl or pyrazol-4-yl, or most preferably pyrazol-3-yl), imidazolyl (preferably imidazol-2-yl), pyrazinyl, pyrimidinyl (preferably pyrimidin-4-yl), pyridazinyl (preferably pyridazin-3-yl), triazolyl (e.g. 1,2,3-triazolyl), or tetrazol-5-yl.

Still more preferably, e.g. in all aspects and/or embodiments of the invention, Het is an optionally substituted monocyclic heteroaryl, attached at a ring-carbon, which is: pyridinyl (preferably pyridin-3-yl or most preferably pyridin-2-yl), pyrazolyl (preferably pyrazol-5-yl or pyrazol-4-yl, or most preferably pyrazol-3-yl), imidazolyl (preferably imidazol-2-yl), pyrazinyl, pyrimidinyl (preferably pyrimidin-4-yl), or pyridazinyl (preferably pyridazin-3-yl).

Yet more preferably, e.g. in all aspects and/or embodiments of the invention, Het is an optionally substituted monocyclic heteroaryl, attached at a ring-carbon, which is: pyridin-3-yl, pyridin-2-yl, pyrazinyl, or pyrazolyl (preferably pyrazol-5-yl or pyrazol-4-yl, or most preferably pyrazol-3-yl).

Most preferably, e.g. in all aspects and/or embodiments of the invention, Het is an optionally substituted monocyclic heteroaryl, attached at a ring-carbon, which is: optionally substituted pyridin-2-yl, optionally substituted pyrazinyl or optionally substituted pyrazol-3-yl.

When Het or Het-CH₂— is present in the compound of formula (I) and Het is a basic heteroaryl group (e.g. pyridinyl, e.g. pyridin-2-yl, or another basic heteroaryl group), then the compound of formula (I) can be optionally present as an agrochemically acceptable salt thereof (and preferably can be present as an agrochemically acceptable acid addition salt thereof such as a hydrohalide, e.g. hydrochloride, salt thereof).

It is particularly preferred (e.g. in all aspects and/or embodiments of the invention) that, in Het, any ring-carbon atom, which is directly bonded to the ring-carbon atom which is the point of attachment (e.g. or i.e. which is the point of attachment to the central carbon atom within the Y═CR⁸R⁹ moiety (for Het), or which is the point of attachment to the —CH₂— moiety (for Het-CH₂—), is unsubstituted. Therefore, for example, preferably, when Het is an optionally substituted pyridin-2-yl (optionally present as an agrochemically acceptable salt thereof), then the ring-carbon atom at the 3-position of the ring (calculated with respect to the pyridine ring nitrogen atom) is unsubstituted.

Preferably, e.g. in all aspects and/or embodiments of the invention, Het is one of the heteroaryls illustrated below:

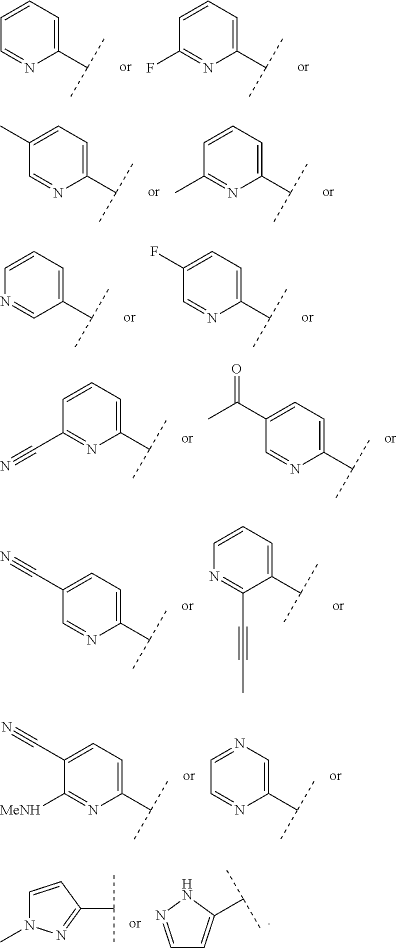

Particularly preferably, e.g. in all aspects and/or embodiments of the invention, Het is one of the heteroaryls illustrated below:

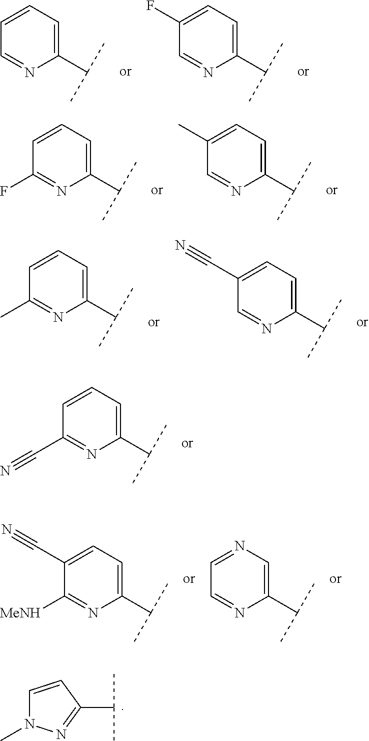

Most preferably, e.g. in all aspects and/or embodiments of the invention, Het is one of the heteroaryls illustrated below:

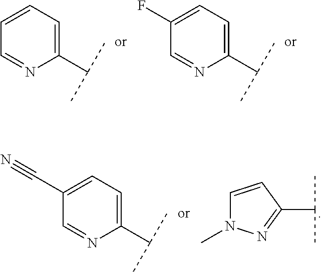

Preferably, e.g. in all aspects and/or embodiments of the invention, R⁷ and R⁸ are both hydrogen.

Preferably, e.g. in all aspects and/or embodiments of the invention, R⁹ is hydrogen or methyl, more preferably hydrogen.

Preferably, e.g. in all aspects and/or embodiments of the invention, R¹⁰ is hydrogen.

Preferably, e.g. in all aspects and/or embodiments of the invention, R¹¹ and R¹², independently of each other, are hydrogen or methyl. More preferably, one or both of R¹¹ and R¹² is or are hydrogen.

Preferably, e.g. in all aspects and/or embodiments of the invention, R¹³ is hydrogen or methyl, more preferably hydrogen.

Preferably, e.g. in all aspects and/or embodiments of the invention, R¹⁴ is hydrogen; and/or, preferably, R¹⁵ is hydrogen or fluorine; and/or, preferably, R¹⁶ is hydrogen, methyl or fluorine; provided that, in each case, R¹⁴, R¹⁵ and R¹⁶ in total contain no more than one carbon atom, and R¹⁴, R¹⁵ and R¹⁶ in total comprise no more than one chlorine.

Preferably, e.g. in all aspects and/or embodiments of the invention, n4 is 2.

Preferably, e.g. in all aspects and/or embodiments of the invention, n5 is 2.

Preferably, e.g. in all aspects and/or embodiments of the invention, R¹⁷ and/or R¹⁸ independently are C₁-C₂alkyl (in particular methyl), C₁fluoroalkyl (in particular trifluoromethyl), or —NR^{19A}R^{20A}.

More preferably, R¹⁷ and/or R¹⁸ independently are methyl or C₁fluoroalkyl (in particular methyl or trifluoromethyl).

Preferably, e.g. in all aspects and/or embodiments of the invention, n18 is 2.

Preferably, e.g. in all aspects and/or embodiments of the invention, R¹⁹ is —C(O)-methyl, —C(O)—C₁fluoroalkyl (in particular —C(O)-trifluoromethyl), —S(O)₂-methyl, —S(O)₂—C₁fluoroalkyl (in particular —S(O)₂-trifluoromethyl), methyl, or C₁fluoroalkyl (in particular trifluoromethyl).

Preferably, e.g. in all aspects and/or embodiments of the invention, R²⁰ and R^{20A} independently are hydrogen, methyl or trifluoromethyl.

Preferably, e.g. in all aspects and/or embodiments of the invention, R^{19A} is hydrogen, methyl or trifluoromethyl.

In one preferred embodiment of the invention, R³ and R⁴ are taken together, and/or R³ and R⁵ are taken together, to form a chain which is (a), (b), (c), (d), (e), (f) or (g), as shown below:

—C(R²¹)(R²²)—C(R²³)(R²⁴)—Y—C(R²⁵)(R²⁶)—C(R²⁷)(R²⁸)— (a)

—C(R²¹)(R²²)—Y—C(R^{21A})(R^{22A})—C(R^{21A})(R^{22A})—C(R²⁷)(R²⁸)— (b)

—CH(R²⁹)—Y—C(R^{21A})(R^{22A})—CH(R³²)— (c)

—CH(R²⁹)—Y—CH(R³²)— (d)

—C(R²¹)(R²²)—C(R²³)(R²⁴)—C(R³⁰)(R³¹)—C(R²⁵)(R²⁶)—C(R²⁷)(R²⁸)— (e)

—C(R²¹)(R²²)—C(R^{21A})(R^{22A})—C(R³⁰)(R³¹)—C(R²⁷)(R²⁸)— (f)

—CH(R²⁹)—C(R³⁰)(R³¹)—CH(R³²)— (g)

wherein Y is O or NR⁴⁰.

R³ and R⁴ being taken together, and/or R³ and R⁵ being taken together, to form a chain is a preferred embodiment of the invention (i.e. is preferred in all aspects and/or embodiments of the invention).

Preferably, when R³ and R⁴ are taken together, and/or R³ and R⁵ are taken together, to form a chain, then the chain is (a), (d), (e), (f) or (g), more preferably (a), (e), (f) or (g), more preferably (a), (e) or (g) or (a), (f) or (g), most preferably (a) or (f), in particular (a), as defined herein.

Preferably, e.g. in all aspects and/or embodiments of the invention, Y is O; and/or R⁴⁰ is C₁-C₃alkoxy such as methoxy.

Preferably, e.g. in all aspects and/or embodiments of the invention, all of R²¹, R²², R²⁷ and R²⁸ are hydrogen. In this preferred embodiment, more preferably Y is O and/or R⁴⁰ is C₁-C₃alkoxy; and/or more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Preferably, e.g. in all aspects and/or embodiments of the invention, R^{21A} and R^{22A} are both hydrogen. In this preferred embodiment, more preferably Y is O and/or R⁴⁰ is C₁-C₃alkoxy; and/or more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Preferably, e.g. in all aspects and/or embodiments of the invention, R²⁹ and R³² are both hydrogen. In this preferred embodiment, more preferably Y is O and/or R⁴⁰ is C₁-C₃alkoxy; and/or more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Preferably, e.g. in all aspects and/or embodiments of the invention, R³⁰ is hydrogen or methoxy, more preferably hydrogen. Preferably, e.g. in all aspects and/or embodiments of the invention, R³¹ is hydrogen. In these preferred embodiments of R³⁰ and/or R³¹, more preferably Y is O and/or R⁴⁰ is C₁-C₃alkoxy; and/or more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Preferably, e.g. in all aspects and/or embodiments of the invention, all of R²³, R²⁴, R²⁵ and R²⁶ are hydrogen. In this preferred embodiment, more preferably Y is O and/or R⁴⁰ is C₁-C₃alkoxy; and/or more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

More preferably, e.g. in all aspects and/or embodiments of the invention, all of R²¹, R²², R²⁷ and R²⁸ are hydrogen, R^{21A} and R^{22A} are both hydrogen, and R²⁹ and R³² are both hydrogen. In this more preferred embodiment, even more preferably, Y is O and/or R⁴⁰ is C₁-C₃alkoxy; and/or even more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Most preferably, e.g. in all aspects and/or embodiments of the invention, all of R²¹, R^{21A}, R²², R^{22A}, R²³, R²⁴, R²⁵, R²⁶, R²⁷, R²⁸, R²⁹, R³⁰, R³¹ and R³² are hydrogen. In this most preferred embodiment, even more preferably, Y is O and/or R⁴⁰ is C₁-C₃alkoxy; and/or even more preferably R³ and R⁴ are taken together, and/or R³ and R⁵ are taken together, to form a chain which is (a), (d), (e), (f) or (g), more preferably (a), (e), (f) or (g), more preferably (a), (e) or (g), most preferably (a) or (e), as defined herein.

Preferably, e.g. in all aspects and/or embodiments of the invention, R⁴⁰ is C₁-C₃alkoxy. More preferably, R⁴⁰ is methoxy.

In a preferable embodiment of the invention (which e.g. can apply to all aspects and/or embodiments of the invention), the compound of formula (I) is a compound as described and/or illustrated in any of Tables 1 to 47 herein (e.g. hereinafter), optionally present (e.g. where chemically possible) as an agrochemically acceptable salt thereof. In one particular embodiment of this embodiment, the compound is one in which R¹ is methyl. In another particular embodiment of this embodiment, the compound is one in which R¹ is methoxy.

In a more preferable embodiment of the invention (which e.g. can apply to all aspects and/or embodiments of the invention), the compound of formula (I) is a compound as described and/or illustrated in any of Tables 1 to 37 herein (e.g. hereinafter), optionally present (e.g. where chemically possible) as an agrochemically acceptable salt thereof. In one particular embodiment of this embodiment, the compound is one in which R¹ is methyl. In another particular embodiment of this embodiment, the compound is one in which R¹ is methoxy.

In a more preferable embodiment of the invention (which e.g. can apply to all aspects and/or embodiments of the invention), the compound of formula (I) is a compound as described and/or illustrated in any of Tables 1, 3, 4, 5, 8, 9, 10, 11, 12, 14, 15, 16, 17, 18, 19, 20, 21, 22, 23, 24, 25, 26, 27, 28, 29, 30, 31, 32, 33, 34, 35, 36 or 37 herein (e.g. hereinafter), optionally present (e.g. where chemically possible) as an agrochemically acceptable salt thereof. In one particular embodiment of this embodiment, the compound is one in which R¹ is methyl. In another particular embodiment of this embodiment, the compound is one in which R¹ is methoxy. In an alternative more preferable embodiment of the invention (which e.g. can apply to all aspects and/or embodiments of the invention), the compound of formula (I) is one of compounds P1 to P7, as described and/or illustrated herein, optionally present (e.g. where chemically possible) as an agrochemically acceptable salt thereof.

In a particularly preferable embodiment of the invention (which e.g. can apply to all aspects and/or embodiments of the invention), the compound of formula (I) is one of compounds A1 to A95, as described and/or illustrated herein, optionally present (e.g. where chemically possible) as an agrochemically acceptable salt thereof. In an alternative particularly preferable embodiment of the invention (which e.g. can apply to all aspects and/or embodiments of the invention), the compound of formula (I) is one of compounds P1 to P7, as described and/or illustrated herein, optionally present (e.g. where chemically possible) as an agrochemically acceptable salt thereof.

In a more particularly preferable embodiment of the invention (which e.g. can apply to all aspects and/or embodiments of the invention), the compound of formula (I) is one of compounds A1 to A93, as described and/or illustrated herein, optionally present (e.g. where chemically possible) as an agrochemically acceptable salt thereof. In an alternative more particularly preferable embodiment of the invention (which e.g. can apply to all aspects and/or embodiments of the invention), the compound of formula (I) is one of compounds P1 to P7, as described and/or illustrated herein, optionally present (e.g. where chemically possible) as an agrochemically acceptable salt thereof.

In one yet more particularly preferable embodiment of the invention (which e.g. can apply to all aspects and/or embodiments of the invention), the compound of formula (I) is one of compounds A1, A2, A4, A5, A6, A7, A9, A10, A13, A14, A16, A17, A18, A19, A21, A22, A23, A24, A26, A27, A28, A29, A31, A32, A33, A34, A36, A37, A38, A39, A41, A42, A43, A44, A46 or A47, or is one of compounds P1, P2, P3, P4, P5, P6 or P7, as described and/or illustrated herein, optionally present (e.g. where chemically possible) as an agrochemically acceptable salt thereof. In one embodiment, these compounds are applied to, or at the locus of, crops of useful plants comprising leguminous crops (in particular soybean, peanut, and/or pulse crops), cotton, rape (in particular oilseed rape or canola), sunflower, sugarbeet, fodder beet, potato, and/or vegetables (in particular dicotyledonous vegetables).

In another yet more particularly preferable embodiment of the invention (which e.g. can apply to all aspects and/or embodiments of the invention), the compound of formula (I) is one of compounds A1, A3, A6, A8, A11, A12, A13, A15, A18, A20, A23, A25, A28, A30, A33, A35, A38, A40, A43, A45, A48, A49, A50, A51, A52, A53, A54, A55, A56, A57, A58, A59, A60, A61, A62, A63, A64, A65, A66, A67, A68, A69, A70, A71, A72, A73, A74, A75, A76, A77, A78, A79, A80, A81, A82, A83, A84, A85, A86, A87, A88, A89, A90, A91, A92 or A93, or is one of compounds P1, P2, P3, P4, P5, P6 or P7, as described and/or illustrated herein, optionally present (e.g. where chemically possible) as an agrochemically acceptable salt thereof. In one embodiment, these compounds are applied to, or at the locus of, crops of useful plants comprising cereals, in particular non-oat cereals, more particularly wheat (e.g. summer or winter wheat, or durum wheat), barley (e.g. summer or winter barley), rye and/or triticale.

Depending on the nature of the substituents G, R¹, R², R³, X, R⁴, R⁵, and R⁶, compounds of formula (I) may exist in different isomeric forms. When G is hydrogen, for example, compounds of formula (I) may exist in different tautomeric forms (A), (AA), and/or (B):

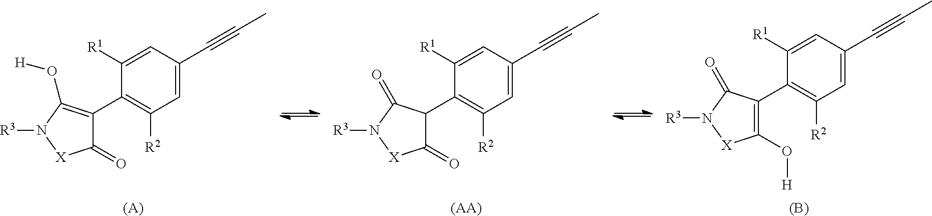

Also, when substituents contain double bonds, cis- and trans-isomers can exist. The compounds of formula (I) of the present invention cover all such tautomers and/or isomers, and mixtures thereof, in all proportions.

Processes for Preparation of Compounds, e.g. Compounds of Formula (I)

The present invention covers a compound of formula (I) (which optionally can be a agrochemically acceptable salt thereof):

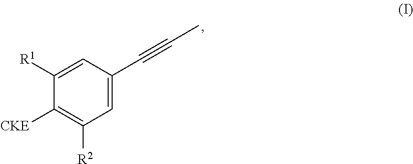

wherein CKE is either of sub-formula (A) or of sub-formula (B):

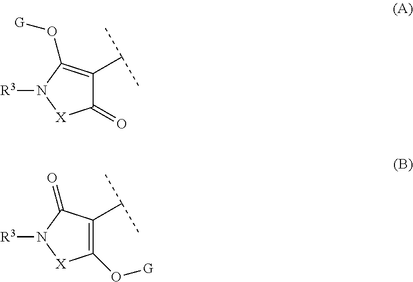

or wherein CKE is a mixture of sub-formulae (A) and (B) e.g. in any molar ratio.

Processes for preparation of a compound of formula (I) (which optionally can be an agrochemically acceptable salt thereof), are now described, and these processes form further aspects of the present invention.

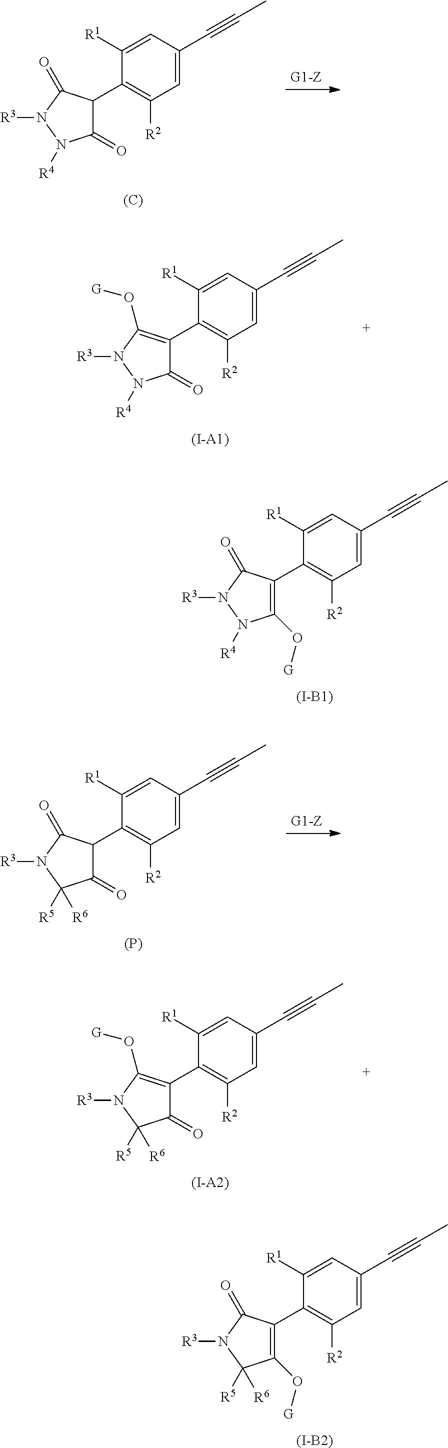

A compound of formula (I), in particular a compound of formula (I-A1) and/or (I-B1) or a compound of formula (I-A2) and/or (I-B2), wherein G is:

—C(X^{a})—R^{a}, —C(X^{b})—X^{c}—R^{b}, —C(X^{d})—N(R^{c})—R^{d}, —SO₂—R^{e}, —P(X^{e})(R^{f})—R^{g}, —CH₂—X^{f}—R^{h}; or phenyl-CH₂— or phenyl-CH(C₁-C₂alkyl)- (in each of which the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or heteroaryl-CH₂— or heteroaryl-CH(C₁-C₂alkyl)- (in each of which the heteroaryl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or phenyl-C(O)—CH₂— (wherein the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro); or C₁-C₆alkoxy-C(O)—CH₂—, C₁-C₆alkoxy-C(O)—CH═CH—, C₂-C₇alken-1-yl-CH₂—, C₂-C₇alken-1-yl-CH(C₁-C₂alkyl)-, C₂-C₄fluoroalken-1-yl-CH₂—, C₂-C₇alkyn-1-yl-CH₂—, or C₂-C₇alkyn-1-yl-CH(C₁-C₂alkyl)-;
may be prepared:
(to prepare a compound of formula (I-A1) and/or (I-B1)) either by treating a compound of formula (C), which is a compound of formula (I-A1) or (I-B1) wherein G is H, or
(to prepare a compound of formula (I-A2) and/or (I-B2)) by treating a compound of formula (P), which is a compound of formula (I-A2) or (I-B2) wherein G is H,
   (a) with a reagent G1-Z, wherein G1-Z is an alkylating agent (wherein G1 is an organic group according to G within the compound of formula (I) and which is linked by a non-carbonyl, non-thiocarbonyl carbon atom) such as an organic halide (in which Z=halogen such as chlorine, bromine or iodine); wherein the organic halide (e.g. chloride) can typically be a substituted alkyl halide (e.g. chloride) such as a chloromethyl alkyl ether Cl—CH₂—X^{f}—R^{h} wherein X^{f} is oxygen, a chloromethyl alkyl sulfide Cl—CH₂—X^{f}—R^{h} wherein X^{f} is sulphur, a suitable optionally substituted benzyl halide (e.g. chloride) such as Cl—CH₂-[optionally substituted phenyl], [optionally substituted phenyl]-C(O)—CH₂-[halogen e.g. Cl], C₁-C₆alkoxy-C(O)—CH₂-[halogen e.g. Cl], C₁-C₆alkoxy-C(O)—CH═CH-[halogen e.g. Cl], a suitable alkenyl or alkynyl halide (e.g. chloride) such as C₂-C₇alken-1-yl-CH₂-[halogen e.g. Cl] or C₂-C₇alkyn-1-yl-CH₂-[halogen e.g. Cl], or another organic halide suitable for preparing a (non-carbonyl, non-thiocarbonyl carbon)-linked G (or G1) group; or
   (b) [e.g. to prepare carbonyl-carbon-linked or thiocarbonyl-carbon-linked G groups] with an acylating agent such as a carboxylic acid, HO—C(X^{a})R^{a}, wherein X^{a} is oxygen, an acid chloride, Cl—C(X^{a})R^{a}, wherein X^{a} is oxygen, or an acid anhydride, [R^{a}C(X^{a})]₂O, wherein X^{a} is oxygen, or an isocyanate, R^{c}N═C═O, or a carbamoyl chloride, Cl—C(X^{d})—N(R^{c})—R^{d} (wherein X^{d} is oxygen and with the proviso that neither R^{c} or R^{d} is hydrogen), or a thiocarbamoyl chloride Cl—(X^{d})—N(R^{c})—R^{d} (wherein X^{d} is sulfur and with the proviso that neither R^{c} or R^{d} is hydrogen), or a chloroformate, Cl—C(X^{b})—X^{c}—R^{b} (wherein X^{b} and X^{c} are oxygen), or a chlorothioformate Cl—C(X^{b})—X^{c}—R^{b} (wherein X^{b} is oxygen and X^{c} is sulfur), or a chlorodithioformate Cl—C(X^{b})—X^{c}—R^{b} (wherein X^{b} and X^{c} are sulfur), or an isothiocyanate, R^{c}N═C═S; or
   (c) by sequential treatment with carbon disulfide and an alkylating agent; or
   (d) with a phosphorylating agent such as a phosphoryl chloride, Cl—P(X^{e})(R^{f})—R^{g}; or
   (e) with a sulfonylating agent such as a sulfonyl chloride Cl—SO₂—R^{e}, preferably in the presence of at least one equivalent of base.

The O-alkylation of cyclic 1,3-diones is known; suitable methods are described, for example, by T. Wheeler, U.S. Pat. No. 4,436,666. Alternative procedures have been reported by M. Pizzorno and S. Albonico, Chem. Ind. (London), (1972), 425-426; H. Born et al., J. Chem. Soc., (1953), 1779-1782; M. G. Constantino et al., Synth. Commun., (1992), 22 (19), 2859-2864; Y. Tian et al., Synth. Commun., (1997), 27 (9), 1577-1582; S. Chandra Roy et al., Chem. Letters, (2006), 35 (1), 16-17; P. K. Zubaidha et al., Tetrahedron Lett., (2004), 45, 7187-7188.

The O-acylation of cyclic 1,3-diones may be effected e.g. by procedures similar to those described, for example, by R. Haines, U.S. Pat. No. 4,175,135, and by T. Wheeler, U.S. Pat. No. 4,422,870, U.S. Pat. No. 4,659,372 and U.S. Pat. No. 4,436,666. Typically diones of formula (A) may be treated with an acylating agent preferably in the presence of at least one equivalent of a suitable base, and optionally in the presence of a suitable solvent. The base may be inorganic, such as an alkali metal carbonate or hydroxide, or a metal hydride, or an organic base such as a tertiary amine or metal alkoxide. Examples of suitable inorganic bases include sodium carbonate, sodium or potassium hydroxide, sodium hydride, and suitable organic bases include trialkylamines, such as trimethylamine and triethylamine, pyridines or other amine bases such as 1,4-diazobicyclo[2.2.2]-octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent. For cases where the acylating agent is a carboxylic acid, acylation is preferably effected in the presence of a known coupling agent such as 2-chloro-1-methylpyridinium iodide, N,N′-dicyclohexylcarbodiimide, 1-(3-dimethylaminopropyl)-3-ethylcarbodiimide and N,N′-carbodiimidazole, and optionally in the presence of a base such as triethylamine or pyridine in a suitable solvent such as tetrahydrofuran, dichloromethane or acetonitrile. Suitable procedures are described, for example, by W. Zhang and G. Pugh, Tetrahedron Lett., (1999), 40 (43), 7595-7598; T. Isobe and T. Ishikawa, J. Org. Chem., (1999), 64 (19), 6984-6988 and K. Nicolaou, T. Montagnon, G. Vassilikogiannakis, C. Mathison, J. Am. Chem. Soc., (2005), 127(24), 8872-8888.

Phosphorylation of cyclic 1,3-diones may be effected e.g. using a phosphoryl halide or thiophosphoryl halide and a base e.g. by procedures analogous to those described by L. Hodakowski, U.S. Pat. No. 4,409,153.

Sulfonylation of a compound of formula (A) may be achieved e.g. using an alkyl or aryl sulfonyl halide, preferably in the presence of at least one equivalent of base, for example by the procedure of C. Kowalski and K. Fields, J. Org. Chem., (1981), 46, 197-201.

Processes for the Preparation of Compounds of Formula (I) in which X═NR⁴, Such as a Compound of Formula (C)

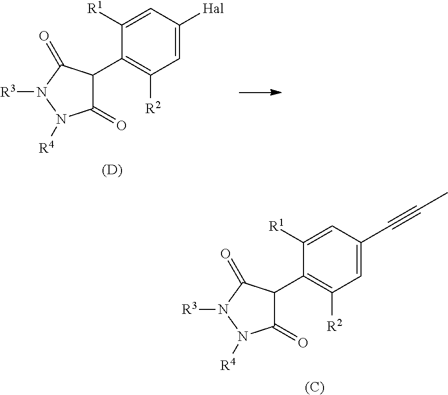

A compound of formula (C) can be prepared by reacting a compound of formula (D) with propyne, preferably in the presence of a suitable catalyst, optionally in the presence of a suitable additive, optionally in a suitable solvent e.g. at a suitable temperature. Suitable catalysts include transition metal salts or complexes of transition metal salts (for example palladium acetate, bis(triphenylphosphine) palladium(II) dichloride, tetrakis(triphenylphosphine)palladium(0), bis(triphenylphosphine) nickel(II) dichloride and tris(acetylacetonato) iron(III)), in an amount typically 0.001-25% with respect to a compound of formula (D). Suitable additives include copper salts (for example copper(I) iodide in an amount typically 0.001-50% with respect to a compound of formula (D)), and tetraalkyl ammonium salts. Suitable bases include diethylamine, triethylamine, piperidine and pyrrolidine, and suitable solvents include 1,4-dioxane, N,N-dimethylacetamide or N,N-dimethylformamide. Preferably the reaction is carried out using 0.05-10% bis(triphenylphosphine) palladium(II) dichloride (with respect to a compound of formula (D)), 0.05-10% triphenylphosphine (with respect to a compound of formula (D)), 0.05-25% copper(I) iodide (with respect to a compound of formula (D)), 5-200% tetrabutyl ammonium iodide (with respect to a compound of formula (D)), triethylamine and N,N-dimethylformamide at a temperature between 25° C. to 150° C. Such a reaction is an example of a Sonogashira coupling and similar reactions are known in the literature (see for example F. Labrie, S. Gauthier, J. Cloutier, J. Mailhot, S. Potvin, S. Dion, J-Y. Sanceau, WO 2008124922; M. S. Viciu, S. P. Nolan, Modern Arylation Methods (2009), 183-220; R. Chinchilla, C. Najera, Chemical Reviews (2007), 107(3), 874-922; I. P. Beletskaya, G. V. Latyshev, A. V. Tsvetkov, N. V. Lukashev, Tetrahedron Letters (2003), 44(27), 5011-5013 and J. Mao, G. Xie, M. Wu, J. Guo, S. Ji, Advanced Synthesis & Catalysis (2008), 350(16), 2477-2482). In an alternative approach a compound of formula (C) may be prepared from a compound of formula (D) by reaction with a propynyl transfer reagent such as 1-propynyllithium, 1-propynylmagnesium bromide, 1-propynylmagnesium chloride, 1-propynylmagnesium iodide, 1-propynylzinc chloride, 1-propynylzinc bromide, 1-propynylzinc iodide, tributylpropynylstannane, 1-propyne-1-boronic acid (or ester thereof), 2-butynoic acid or 1-(trimethylsilyl)propyne, with a transition metal catalyst system under suitable conditions (see for example P. Wessig, G. Mueller, C. Pick, A. Matthes, Synthesis (2007), (3), 464-477; J. H. Chaplin, G. S. Gill, D. W. Grobelny, B. L. Flynn, G. Kremmidiotis, WO07087684; A. Akao, T. Tsuritani, S. Kii, K. Sato, N. Nonoyama, T. Mase, N. Yasuda, Synlett (2007), (1), 31-36. A. Coelho Coton, E. Sotelo Perez, F. Guitian Rivera, A. Gil Gonzalez, WO 2011048247; C. H. Oh, S. H. Jung, Tetrahedron Letters (2000), 41(44), 8513-8516; D. Zhao, C. Gao, X. Su, Y. He, J. You, Y. Xue, Chemical Communications (2010), 46(47), 9049-9051; C. Yang, S. P. Nolan, Organometallics (2002), 21(6), 1020-1022). In another set of preferred conditions a compound of formula (D) is reacted with 1-propynylmagnesium bromide in the presence of 0.05-10% bis(triphenylphosphine) palladium(II) dichloride (with respect to a compound of formula (D)), in tetrahydrofuran at a temperature between 25° C. and 100° C., as described by J. H. Chaplin, G. S. Gill, D. W. Grobelny, B. L. Flynn, G. Kremmidiotis, WO 07087684.

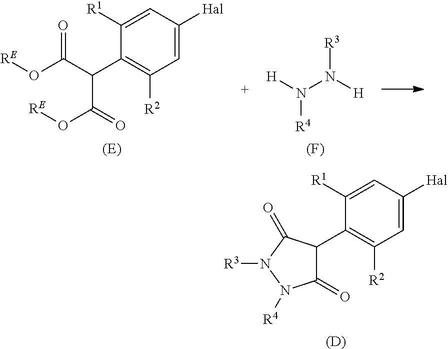

A compound of formula (D) can be prepared by reacting a compound of formula (E) wherein each R^{E} is independently C₁-C₆alkyl (preferably independently methyl or ethyl), and a hydrazine of formula (F), preferably by treatment in a suitable solvent, e.g. at a suitable temperature (including microwave heating), with or without the addition of a suitable base. Suitable solvents include alcohols, such as methanol or ethanol, tetrahydrofuran, or high boiling solvents such as toluene or xylene. Suitable bases, in greater than or sub-stoichiometric amounts, can be metal alkoxides, such as sodium methoxide, potassium methoxide, sodium ethoxide, potassium ethoxide, sodium tert-butoxide or potassium tert-butoxide, or organic amine bases such as 1,8-diazabicyclo[5.4.0]-undec-7-ene, triethylamine or diisopropylethylamine. Suitable temperatures can be from cooled below 0° C. up to the boiling point of the solvent used. Similar reactions are known in the literature (see for example M. Muehlebach et al, Bioorganic and Medicinal Chemistry 2009, 17(12), 4241, Sherif A. F. Rostom, Bioorganic and Medicinal Chemistry 2010, 18(7), 2767, K. H. Dudley et al, Journal of Heterocyclic Chemistry 1978, 15(6), 923). Hydrazines of formula (F) are known in the literature or can be prepared from known reagents using known methods.

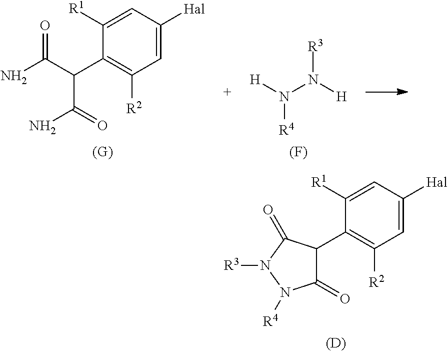

A compound of formula (D) can alternatively be prepared by reacting a compound of formula (G) and a hydrazine of formula (F), preferably by treatment in a suitable solvent, e.g. at a suitable temperature (including microwave heating), preferably with a suitable base. Suitable solvents include high boiling solvents such as toluene or xylene. Suitable bases, in greater than or sub-stoichiometric amounts, can be organic amine bases such as 1,8-diazabicyclo[5.4.0]-undec-7-ene, triethylamine or diisopropylethylamine. Suitable temperatures can be from cooled below 0° C. up to the boiling point of the solvent used. Similar reactions are known in the literature (see for example Maetzke, Thomas et al, WO00/078881).

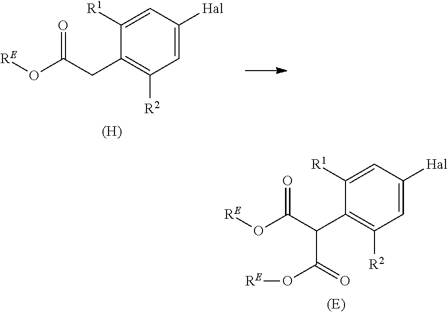

A compound of formula (E) can be prepared by reacting a compound of formula (H), wherein R^{E} is C₁-C₅alkyl (preferably methyl or ethyl), and a carboxylic acid or ester equivalent, preferably by treatment in a suitable solvent, e.g. at a suitable temperature, preferably with a suitable base. Suitable solvents include, for example, 1,2-dimethoxyethane, tetrahydrofuran, 1,4-dioxane, diethyl ether, dibutyl ether, acetonitrile, dimethyl sulfoxide, N, N-dimethylformamide, benzene, toluene, methanol, ethanol, isopropanol or tert-butanol, and is chosen to be compatible with the base under the reaction conditions. Suitable bases include metal alkoxides, such as sodium methoxide, sodium ethoxide, potassium tert-butoxide or sodium tert-butoxide, metal hydrides, such as sodium hydride or potassium hydride, alkyl metals, such as n-butyl lithium, or metal amides, such as lithium diisopropylamide, lithium diisopropylamide, lithium hexamethyldisilazide or lithium 2,2,6,6-tetramethylpiperidide. Suitable temperatures can be in a temperature range of −100° C. up to the boiling point of the solvent used. Suitable carboxylic acid or ester equivalents are known or can be prepared from known reagents using known methods (see for example Behera, Manoranjan et al, Tetrahedron Letters (2009) 53(9), 1060, Volonterio, Alessandro and Zanda, Matteo, Organic Letters (2007), 9(5) 841, Levene, P. A. and Meyer, G. M., Organic Syntheses (1936) No. 16, Shintani, Ryo et al, Organic Letters (2009) 11(2), 457, Matulenko, Mark A. et al, Bioorganic and Medicinal Chemistry (2007), 15(4), 1586) and may include carbon dioxide, chloroformates, for example methyl chloroformate or ethyl chloroformate, cyanoformates, such as methyl cyanoformate or ethyl cyanoformate, carbonates, for example dimethyl carbonate or diethyl carbonate, oxalates, such as diethyloxalate or dimethyloxalate, other equivalents include, for example, ethyl-1-imidazole carbamate. Compounds of formula (H) are known in the literature or can be prepared from known reagents using known methods.

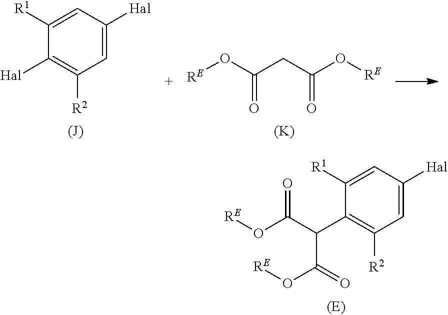

A compound of formula (E), wherein each R^{E} is independently C₁-C₆alkyl (preferably independently methyl or ethyl), can alternatively be prepared by reacting a compound of formula (J) and a compound of formula (K), wherein each R^{E} is independently C₁-C₆alkyl (preferably independently methyl or ethyl), preferably by treatment in a suitable solvent, e.g. at a suitable temperature (including microwave heating), optionally with a suitable base, and preferably in the presence of a suitable catayst system. Suitable solvents include, for example, 1,2-dimethoxyethane, tetrahydrofuran, 1,4-dioxane, acetonitrile, dimethyl sulfoxide, N, N-dimethylformamide, benzene, toluene, 1-methyl-2-pyrrolidone or N, N-dimethylacetamide. Suitable bases, in greater than or sub-stoichiometric amounts, can be metal carbonates, such as potassium carbonate and cesium carbonate, metal hydroxides, such as barium hydroxide, metal hydrides, such as sodium hydride, or metal phosphates, such as tripotassium phosphate. Suitable catalyst systems include using metal salts and complexes of metal salts, such as copper (I) iodide, ferric acetylacetonate, palladium acetate, palladium (II) chloride, bis(triphenylphosphine) palladium(II) dichloride, tetrakis(triphenylphosphine)palladium(0), sodium tetrachloropalladate, with or without additives, such as organo phosphines, such as triphenyl phosphine or t-butylphosphine and salts thereof, copper (II) oxide or organic carboxylates, such as (S)-proline or 2-pyridine carboxylic acid. Similar reactions are known in the literature (for example Reddy Chidipudi, Suresh et al, Angewandte Chemie International Edition (2012) 51(48) 12115, Breslow, Ronald et al, WO2011146855, Yip, Sau Fan et al, Organic Letters (2007), 9(17), 3469, Aramaendia, Maria A. et al, Tetrahedron Letters (2002), 43(15), 2847, Taillefer, Marc et al, WO2008004088. Compounds of formula (J) are known or can be prepared from known reagents using known methods. Compounds of formula (K) are known, for example dimethyl malonate or diethyl malonate, or can be prepared from known reagents using known methods.

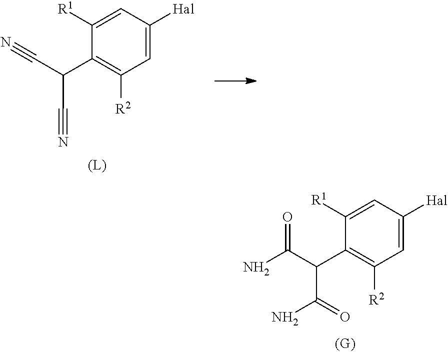

A compound of formula (G) can be prepared by treatment of a compound of formula (L) with an acid (e.g a suitable acid), preferably in a suitable solvent, e.g. at a suitable temperature. Suitable solvents include water. Suitable acids are mineral acids, for example concentrated sulfuric acid. Suitable temperatures can be from cooled below 0° C. up to the boiling point of the solvent used. Similar reactions are known in the literature (see for example Maetzke, Thomas et al, WO00/078881).

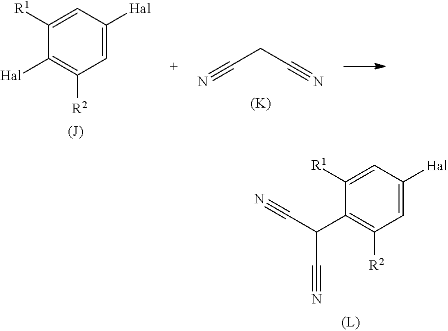

A compound of formula (L) can be prepared by reacting a compound of formula (J) and malononitrile (CAS Reg. No. 109-77-3), for example using methods similar to those previously described. Similar reactions are known in the literature (see for example Zeller, Martin, WO2004/050607; Schnyder, Anita et al, Synlett (2006), 18, 3167; Gao, Chengwei et al, Synlett (2003) 11, 1716; Makosza, Mieczyslaw and Chesnokov, Alexey, Tetrahedron (2008), 64(25), 5925).

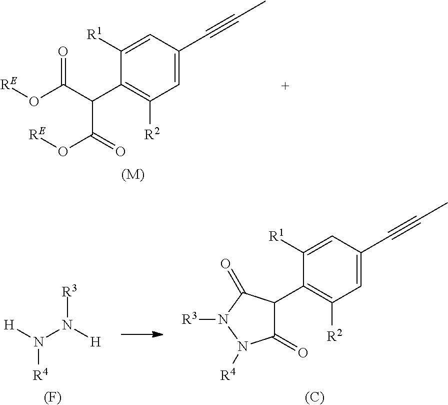

Compounds of formula (C) can also be prepared by reacting compounds of formula (M) wherein each R^{E} is independently C₁-C₆alkyl (preferably independently methyl or ethyl), with hydrazines of formula (F), for example under conditions as previously described.

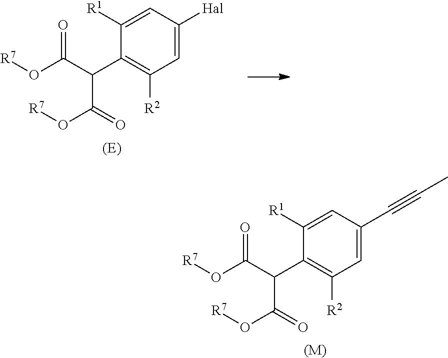

Compounds of formula (M) can be prepared from compounds of formula (E), e.g. under conditions as previously described e.g. in the preparation of a compound of formula (C) from a compound of formula (D).

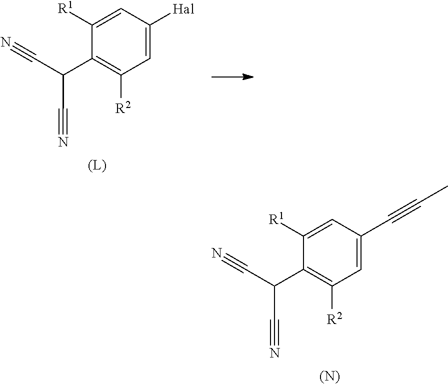

Compounds of formula (N) can be prepared from compounds of formula (L), e.g. under conditions as previously described e.g. in the preparation of a compound of formula (C) from a compound of formula (D).

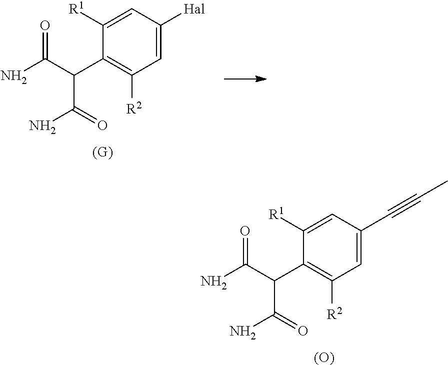

Compounds of formula (0) can be prepared from compounds of formula (G), e.g. under conditions as previously described e.g. in the preparation of a compound of formula (C) from a compound of formula (D).

Processes for the Preparation of Compounds of Formula (I) in which X═CR⁵R⁶, Such as a Compound of Formula (P)

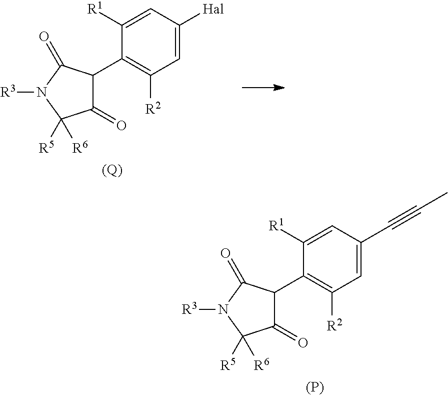

A compound of formula (P) can be prepared from a compound of formula (Q), in particular under conditions as previously described (i.e. as described in the section entitled “Processes for the preparation of compounds of formula (I) in which X═NR⁴, such as a compound of formula (C)”).

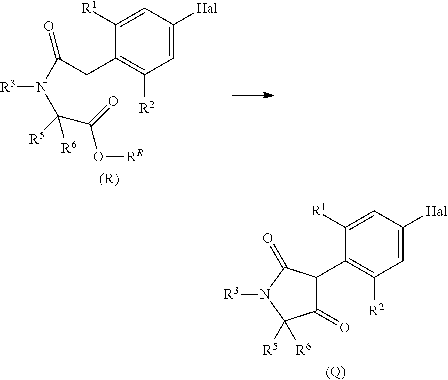

A compound of formula (Q) can be prepared by treatment of a compound of formula (R) wherein R^{R} is C₁-C₆alkyl (preferably methyl or ethyl), with a base (e.g. a suitable base), preferably in a suitable solvent, e.g. at a suitable temperature. The base may be inorganic such as an alkali metal carbonate or hydroxide or a metal hydride, or an organic base such as metal alkoxide. Suitable solvents for this reaction are selected to be compatible with the reagents and include toluene, ethers, such as tetrahydrofuran and 1,2-dimethoxyethane and halogenated solvents, such as dichloromethane and chloroform. Similar reactions are known in the literature (see for example WO2009/049851).

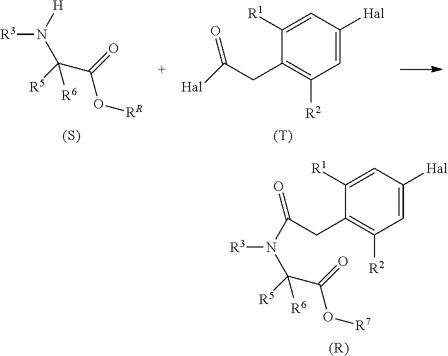

A compound of formula (R) can be prepared by reacting a compound of formula (S), wherein R^{R} is C₁-C₆alkyl (preferably methyl or ethyl), and a compound of formula (T) in the presence of base (e.g. triethylamine), preferably in a suitable solvent. Suitable solvents for this reaction are selected to be compatible with the reagents and include ethers, such as tetrahydrofuran and 1,2-dimethoxyethane, and halogenated solvents, such as dichloromethane and chloroform. Similar reactions are known in the literature (see for example WO2013/079672).

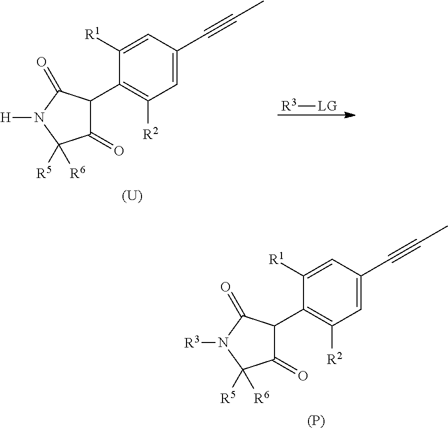

A compound of formula (P) can be prepared by reacting a compound of formula (U) with reagent R3-LG, where in LG represents a leaving group (e.g. halogen), in the presence of base in a suitable solvent. The base may be inorganic, such as a metal hydride, or an organic base, such as metal alkoxide. Suitable solvents for this reaction are selected to be compatible with the reagents and include toluene, ethers, such as tetrahydrofuran and 1,2-dimethoxyethane, and halogenated solvents, such as dichloromethane and chloroform.

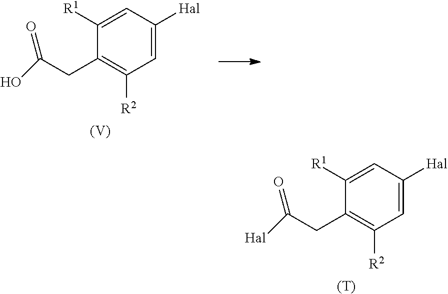

A compound of formula (T) can be prepared by reacting a compound of formula (V) with a suitable halogenating reagent (preferably phosphorous oxychloride, thionyl chloride or oxalyl chloride), preferably in a suitable solvent (e.g. dichloromethane). Compounds of formula (V) are known in the literature or can be prepared from known reagents using known methods.

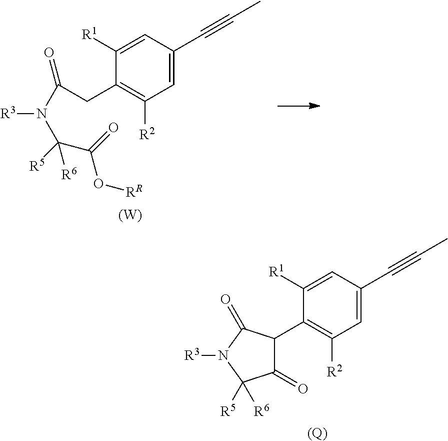

Compounds of formula (P) can be prepared from a compound of formula (W), wherein R^{R} is C₁-C₆alkyl (preferably methyl or ethyl), in particular under conditions as previously described.

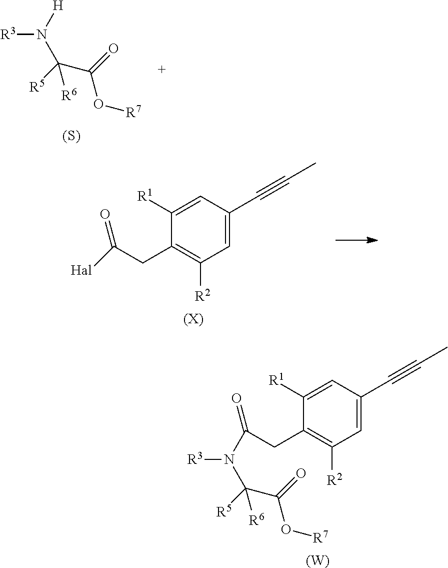

Compounds of formula (W) can be prepared from a compound of formula (S) and a compound of formula (X), wherein Hal is a halogen (preferably chlorine) in particular under conditions as previously described.

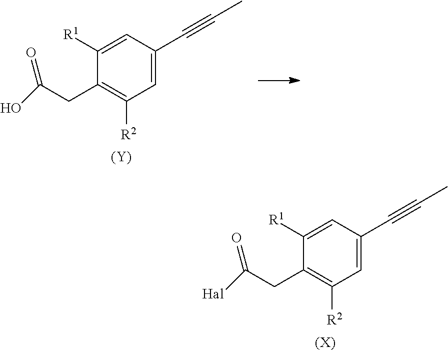

Compounds of formula (X) can be prepared from a compound of formula (Y), in particular under conditions as previously described.

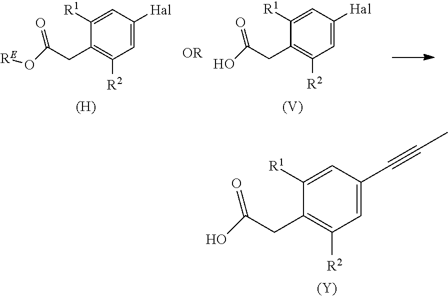

Compounds of formula (Y) can be prepared:

either (i) from a compound of formula (H) (by replacing the halogen group on the phenyl with a prop-1-ynyl group, in particular under conditions as previously described, and then hydrolysing the ester to the carboxylic acid e.g. under conventional ester hydrolysis conditions),
or (ii) from a compound of formula (V), in particular under conditions as previously described.

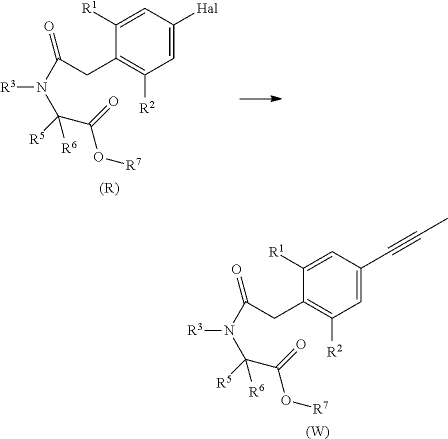

Compounds of formula (W) can be prepared from a compound of formula (R), in particular under conditions as previously described.

Further Processes for the Preparation of Compounds of Formula (I) in which X═NR⁴, Such as a Compound of Formula (C)

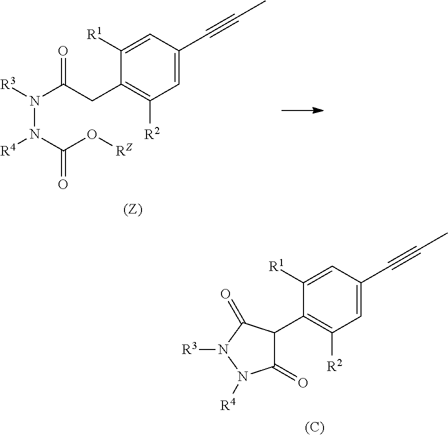

A compound of formula (C) can be prepared by treatment of a compound of formula (Z), wherein R^{Z} is C₁-C₆alkyl (preferably methyl or ethyl), with a base (e.g. a suitable base), preferably in a suitable solvent, e.g. at a suitable temperature and/or as described previously.

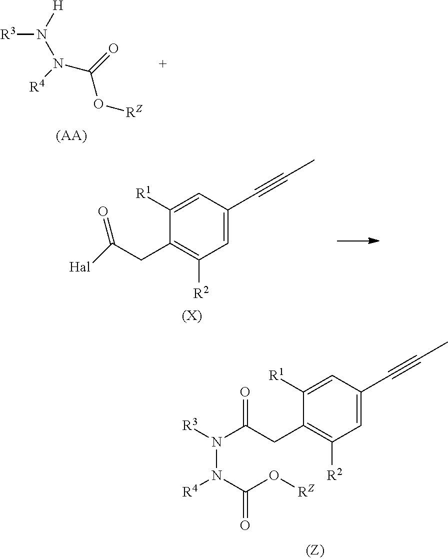

A compound of formula (Z) can be prepared by treatment of a compound of formula (AA), wherein R^{Z} is C₁-C₆alkyl (preferably methyl or ethyl), with a compound of formula (X), wherein Hal is a halogen (preferably chlorine), e.g. using conditions previously described. Compounds of formula (AA) are known in the literature or can be prepared from known reagents using known methods.

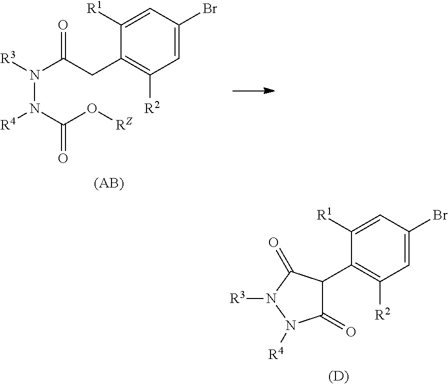

A compound of formula (D) can be prepared by treatment of a compound of formula (AB), wherein R^{Z} is C₁-C₆alkyl (preferably methyl or ethyl), with a base (e.g. a suitable base), preferably in a suitable solvent, e.g. at a suitable temperature and/or as described previously.

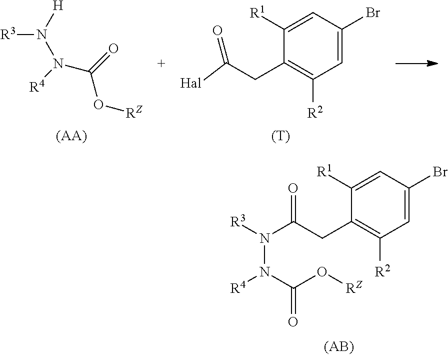

A compound of formula (AB) can be prepared by treatment of a compound of formula (AA), wherein R^{Z} is C₁-C₆alkyl (preferably methyl or ethyl), with a compound of formula (T), wherein Hal is a halogen (preferably chlorine), e.g. using conditions previously described.

### Herbicidal Compositions

In another aspect, the present invention provides a herbicidal composition, e.g. for use in a method of controlling weeds (e.g. preferably monocotyledonous weeds such as more preferably grassy monocotyledonous weeds) in crops of useful plants, which composition comprises a compound of formula (I) as defined herein (e.g. a herbicidally effective amount thereof), and a substantially-inert agrochemically acceptable substance (e.g. an agrochemically acceptable carrier, diluent and/or solvent, an agrochemically acceptable adjuvant, an agrochemically acceptable emulsifier/surfactant/surface-active substance, and/or another agrochemically acceptable additive).

In a further aspect, the present invention provides a herbicidal composition, e.g. for use in a method of controlling weeds (preferably monocotyledonous such as more preferably grassy monocotyledonous weeds) in crops of useful plants, comprising a compound of formula (I) as defined herein (e.g. a herbicidally effective amount thereof), and an agrochemically acceptable carrier, diluent and/or solvent.

In all aspects of the invention, the compound of the formula (I) is optionally present (e.g. where chemically possible) as an agrochemically acceptable salt thereof.

The compounds of formula (I) according to the invention can be used as crop protection agents in unmodified form, as obtained by synthesis, but, for use as herbicides, they are generally formulated into herbicidal compositions (formulations), e.g. in a variety of ways, containing one or more substantially-inert agrochemically acceptable substances (e.g. an agrochemically acceptable carrier, diluent and/or solvent, an agrochemically acceptable adjuvant, an agrochemically acceptable emulsifier/surfactant/surface-active substance, and/or another agrochemically acceptable additive).

The formulations (herbicidal compositions) can be in various physical forms, for example in the form of dusting powders, gels, wettable powders, coated or impregnated granules for manual or mechanical distribution on target sites, water-dispersible granules, water-soluble granules, emulsifiable granules, water-dispersible tablets, effervescent compressed tablets, water-soluble tapes, emulsifiable concentrates, microemulsifiable concentrates, oil-in-water (EW) or water-in-oil (WO) emulsions, other multiphase systems such as oil/water/oil and water/oil/water products, oil flowables, aqueous dispersions, oily dispersions, suspoemulsions, capsule suspensions, soluble liquids, water-soluble concentrates (with water or a water-miscible organic solvent as carrier), impregnated polymer films or in other forms known, for example, from the Manual on Development and Use of FAO Specifications for Plant Protection Products, 5th Edition, 1999. The active ingredient may be incorporated into microfibers or micro-rods formed of polymers or polymerizable monomers and having diameter of about 0.1 to about 50 microns and aspect ratio of between about 10 and about 1000.

Such formulations (herbicidal compositions) can either be used directly or are diluted prior to use. They can then be applied through suitable ground or aerial application spray equipment or other ground application equipment such as central pivot irrigation systems or drip/trickle irrigation means.

Diluted formulations can be prepared, for example, with water, liquid fertilisers, micro-nutrients, biological organisms, oil or solvents.

The formulations (herbicidal compositions) can be prepared, for example, by mixing the active ingredient with formulation adjuvants in order to obtain compositions in the form of finely divided solids, granules, solutions, dispersions or emulsions. The active ingredients can also be contained in fine microcapsules consisting of a core and a polymeric shell. Microcapsules usually have a diameter of from 0.1 to 500 microns. They contain active ingredients in an amount of about from 25 to 95% by weight of the capsule weight. The active ingredients can be present in the form of liquid technical material, in the form of a suitable solution, in the form of fine particles in solid or liquid dispersion or as a monolithic solid. The encapsulating membranes comprise, for example, natural and synthetic gums, cellulose, styrene-butadiene copolymers or other similar suitable membrane forming material, polyacrylonitrile, polyacrylate, polyester, polyamides, polyureas, polyurethane, aminoplast resins or chemically modified starch or other polymers that are known to the person skilled in the art in this connection.

Alternatively it is possible for fine so called “microcapsules” to be formed wherein the active ingredient is present in the form of finely divided particles in a solid matrix of a base substance, but in that case the microcapsule is not encapsulated with a diffusion limiting membrane as outlined in the preceding paragraph.

The active ingredients may be adsorbed on a porous carrier. This may enable the active ingredients to be released into their surroundings in controlled amounts (e.g. slow release). Other forms of controlled release formulations are granules or powders in which the active ingredient is dispersed or dissolved in a solid matrix consisting of a polymer, a wax or a suitable solid substance of lower molecular weight. Suitable polymers are polyvinyl acetates, polystyrenes, polyolefins, polyvinyl alcohols, polyvinyl pyrrolidones, alkylated polyvinyl pyrrolidones, copolymers of polyvinyl pyrrolidones and maleic anhydride and esters and half-esters thereof, chemically modified cellulose esters like carboxymethyl cellulose, methyl cellulose, hydroxyethyl cellulose, examples of suitable waxes are polyethylene wax, oxidized polyethylene wax, ester waxes like montan waxes, waxes of natural origin like carnauba wax, candelilla wax, bees wax etc. Other suitable matrix materials for slow release formulations are starch, stearin, or lignin.

The formulation ingredients (e.g. inert ingredients) suitable for the preparation of the compositions according to the invention are generally known per se.

As a liquid carrier and/or solvent (e.g. organic solvent), e.g. for use in the herbicidal composition(s) according to the invention, there may be used: water, an aromatic solvent such as toluene, m-xylene, o-xylene, p-xylene or a mixture thereof, cumene, an aromatic hydrocarbon blend with a boiling range between 140 and 320° C. (e.g. known under various trademarks such as Solvesso®, Shellsol A®, Caromax®, Hydrosol®), a paraffinic or isoparaffinic carrier such as paraffin oil, mineral oil, a de-aromatized hydrocarbon solvent with a boiling range between 50 and 320° C. (e.g. known for instance under the trademark Exxsol®), a non-dearomatized hydrocarbon solvent with a boiling range between 100 and 320° C. (e.g. known under the tradename Varsol®), an isoparaffinic solvent with a boiling range between 100 and 320° C. (e.g. known under tradenames like Isopar® or Shellsol T®), a hydrocarbon such as cyclohexane, tetrahydronaphthalene (tetralin), decahydronaphthalene, alpha-pinene, d-limonene, hexadecane, isooctane; an ester solvent such as ethyl acetate, n- or iso-butyl acetate, amyl acetate, i-bornyl acetate, 2-ethylhexyl acetate, a C₆-C₁₈ alkyl ester of acetic acid (e.g. known under the tradename Exxate®), lactic acid ethylester, lactic acid propylester, lactic acid butylester, benzyl benzoate, benzyl lactate, dipropyleneglycol dibenzoate, or a dialkyl ester of succinic, maleic or fumaric acid; a polar solvent such as N-methyl pyrrolidone, N-ethyl pyrrolidone, C₃-C₁₈-alkyl pyrrolidones, gamma-butyrolactone, dimethylsulfoxide, N,N-dimethylformamide, N,N-dimethylacetamide, N,N-dimethyllactamide, a C₄-C₁₈ fatty acid dimethylamide, benzoic acid dimethylamide, acetonitrile, acetone, methyl ethyl ketone, methyl-isobutyl ketone, isoamyl ketone, 2-heptanone, cyclohexanone, isophorone, methyl isobutenyl ketone (mesityl oxide), acetophenone, ethylene carbonate, propylene carbonate, or butylene carbonate;

an alcoholic solvent or diluent such as methanol, ethanol, propanol, n- or iso-butanol, n- or iso-pentanol, 2-ethyl hexanol, n-octanol, tetrahydrofurfuryl alcohol, 2-methyl-2,4-pentanediol, 4-hydroxy-4-methyl-2-pentanone, cyclohexanol, benzyl alcohol, ethylene glycol, ethylene glycol butyl ether, ethylene glycol methyl ether, diethylene glycol, diethylene glycol butyl ether, diethylene glycol monoethyl ether, diethylene glycol monomethyl ether, propylene glycol, dipropylene glycol, dipropylene glycol monomethyl ether, or another similar glycol monoether solvent based on a ethylene glycol, propylene glycol or butylene glycol feedstock, triethylene glycol, polyethylene glycol (e.g. PEG 400), a polypropylenglycol with a molecular mass of 400-4000, or glycerol;
glycerol acetate, glycerol diacetate, glycerol triacetate, 1,4-dioxane, diethylene glycol abietate, chlorobenzene, chlorotoluene; a fatty acid ester such as methyl octanoate, isopropyl myristate, methyl laurate, methyl oleate, a mixture of C₈-C₁₀ fatty acid methyl esters, rapeseed oil methyl ester, rapeseed oil ethyl ester, soybean oil methyl ester, soybean oil ethyl ester; a vegetable oil (e.g. rapeseed oil or soybean oil); a fatty acid such as oleic acid, linoleic acid, or linolenic acid; or an ester of phosphoric or phosphonic acid such as triethyl phosphate, a C₃-C₁₈-tris-alkyl phosphate, an alkylaryl phosphate, or bis-octyl-octyl phosphonate.

Water is generally the liquid carrier of choice for the dilution of the concentrates.

Suitable solid carriers are, for example, talc, titanium dioxide, pyrophyllite clay, silica (fumed or precipated silica and optionally functionalised or treated, for instance silanised), attapulgite clay, kieselguhr, limestone, calcium carbonate, bentonite, calcium montomorillonite, cottonseed husks, wheatmeal, soybean flour, pumice, wood flour, ground walnut shells, lignin and similar materials, as described, for example, in the EPA CFR 180.1001. (c) & (d). Powdered or granulated fertilisers can also be used as solid carriers.

A large number of surface-active substances can advantageously be used both in solid and in liquid formulations (herbicidal compositions), especially in those formulations (herbicidal compositions) which can be diluted with a carrier prior to use. Surface-active substances may be anionic, cationic, amphoteric, non-ionic or polymeric and they may be used as emulsifiying, wetting, dispersing or suspending agents or for other purposes. Typical surface-active substances include, for example, salts of alkyl sulfates, such as diethanolammonium lauryl sulfate; or sodium lauryl sulfate, salts of alkylarylsulfonates, such as calcium or sodium dodecylbenzenesulfonate; alkylphenol-alkylene oxide addition products, such as nonylphenol ethoxylates; alcohol-alkylene oxide addition products, such as tridecyl alcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono- and di-alkyl phosphate esters; and also further substances described e.g. in “McCutcheon's Detergents and Emulsifiers Annual”, MC Publishing Corp., Ridgewood, N.J., 1981.

Further formulation ingredients (e.g. inert ingredients) which can typically be used in formulations (herbicidal compositions) include crystallisation inhibitors, viscosity-modifying substances, suspending agents, dyes, anti-oxidants, foaming agents, light absorbers, mixing aids, anti-foams, complexing agents, neutralising or pH-modifying substances and/or buffers, corrosion-inhibitors, fragrances, wetting agents, absorption improvers, micronutrients, plasticisers, glidants, lubricants, dispersants, thickeners, anti-freezes, microbiocides, compatibility agents and/or solubilisers; and/or also liquid and/or solid fertilisers.

The compositions (formulations) may also comprise additional active substances, for example further herbicides, herbicide safeners, plant growth regulators, fungicides or insecticides.

The compositions according to the invention can additionally include an additive (commonly referred to as an adjuvant), comprising a mineral oil, an oil of vegetable or animal origin, alkyl (e.g. C₁-C₆alkyl) esters of such oils or mixtures of such oils and oil derivatives/oil esters. The amount of oil additive (oil adjuvant) used in the composition according to the invention is generally from 0.01 to 10%, based on the spray mixture. For example, the oil additive (oil adjuvant) can be added to the spray tank in the desired concentration after the spray mixture has been prepared. Preferred oil additives (oil adjuvants) comprise mineral oils or an oil of vegetable origin, for example rapeseed oil, olive oil or sunflower oil, emulsifiable vegetable oil, such as AMIGO® (Loveland Products Inc.), C₁-C₆alkyl esters of oils of vegetable origin, for example the methyl esters, or an oil of animal origin, such as fish oil or beef tallow. A preferred oil additive (oil adjuvant) contains methylated rapeseed oil (rapeseed oil methyl ester). Another preferred oil additive (oil adjuvant) contains, for example, as active components essentially 80% by weight alkyl esters of fish oils and 15% by weight methylated rapeseed oil (rapeseed oil methyl ester), and also 5% by weight of customary emulsifiers and pH modifiers. Especially preferred oil additives (oil adjuvants) comprise C₁-C₆alkyl ester(s) of C₈-C₂₂ fatty acid(s), especially the methyl ester(s) of C₈-C₂₂ (especially C₁₂-C₁₈) fatty acid(s); preferably the methyl ester of lauric acid, of palmitic acid, or of oleic acid. Those esters are known as methyl laurate (CAS-111-82-0), methyl palmitate (CAS-112-39-0) and methyl oleate (CAS-112-62-9) respectively. A preferred fatty acid methyl ester derivative is AGNIQUE ME 18 RD-F® (e.g. available from Cognis). Those and other oil derivatives are also known from the Compendium of Herbicide Adjuvants, 5th Edition, Southern Illinois University, 2000.

The application and action of the above-mentioned oil additives (oil adjuvants) can be further improved by combining them with surface-active substances, such as non-ionic, anionic, cationic or amphoteric surfactants. Examples of suitable anionic, non-ionic, cationic or amphoteric surfactants, e.g. for this purpose, are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. As non-ionic surfactants, special preference is given to ethoxylated C₁₂-C₂₂ fatty alcohols preferably having a degree of ethoxylation of from 5 to 40. Examples of commercially available surfactants are the Genapol types (Clariant). Also preferred are silicone surfactants, especially polyalkyl-oxide-modified heptamethyltrisiloxanes, which are commercially available e.g. as SILWET L-77®, and also perfluorinated surfactants. The concentration of surface-active substances in relation to the total oil additive (oil adjuvant) is generally from 1 to 50% by weight of the oil additive (oil adjuvant). Examples of oil additives (oil adjuvants) that consist of mixtures of oils and/or mineral oils and/or derivatives thereof with surfactants are TURBOCHARGE®, ADIGOR® (both (Syngenta Crop Protection AG), ACTIPRON® (BP Oil UK Limited), AGRI-DEX® (Helena Chemical Company).

The above-mentioned surface-active substances may also be used in the formulations alone, that is to say without oil additives (oil adjuvants).

Furthermore, the addition of an organic solvent to the oil additive (oil adjuvant)/surfactant mixture can contribute to a further enhancement of action. Suitable solvents are, for example, heavy aromatic hydrocarbon solvents such as SOLVESSO® or AROMATIC® solvents (Exxon Corporation). The concentration of such solvents can typically be e.g. from 10 to 80% by weight of the oil additive (oil adjuvant). Such oil additives (oil adjuvants), which may be in admixture with solvents, are described, for example, in U.S. Pat. No. 4,834,908. A commercially available oil additive (oil adjuvant) disclosed therein is known by the name MERGE® (BASF). Further such oil additives (oil adjuvants) that are preferred according to the invention are SCORE® and ADIGOR® (both Syngenta Crop Protection AG).

In addition to the oil additives (oil adjuvants) listed above, in order to enhance the activity of the compositions according to the invention it is also possible for formulations of alkylpyrrolidones, (e.g. AGRIMAX® from ISP) to be added to the spray mixture. Formulations of synthetic latices, such as, for example, polyacrylamide, polyvinyl compounds or poly-1-p-menthene (e.g. BOND®, COURIER® or EMERALD®) can also be used.

A particularly preferred oil adjuvant (oil additive), e.g. for use in the herbicidal compositions of the invention, is an emulsifiable concentrate which consists of:

(i) ethoxylated alcohols, which preferably includes ethoxylated C₁₂-C₂₂ fatty alcohols (preferably having a degree of ethoxylation of from 5 to 40); and
(ii) a mixture of heavy aromatic hydrocarbons, which preferably includes (or more preferably includes 50% or more by weight of the heavy aromatic hydrocarbons of) a mixture of naphthalenes each of which is substituted by one or more alkyls wherein the alkyl(s) in total have 1-4 carbon atoms per naphthalene molecule (e.g. Solvesso 200 ND™); and
(iii) methylated rapeseed oil (rapeseed oil methyl ester) (e.g. Agnique ME 18 RD-F™), as an adjuvant; preferably present at about 47% w/w and/or about 45% w/v of the oil adjuvant/oil additive/emulsifiable concentrate. One example of such a emulsifiable concentrate oil adjuvant (oil additive) is ADIGOR™, currently available in many countries from Syngenta.

When the above emulsifiable concentrate oil adjuvant is used, it is preferably added to the herbicidal composition after dilution (e.g. with water and/or in a spray tank), typically before application to weeds and/or to crops of useful plants and/or to the locus thereof. In one particular embodiment, the herbicidal composition, e.g. after dilution (e.g. with water and/or in a spray tank), contains the above emulsifiable concentrate oil adjuvant, and additionally ammonium sulphate and/or isopropyl alcohol.

Such adjuvant oils as described in the preceding paragraphs may be employed as the carrier liquid in which an active compound is dissolved, emulsified or dispersed as appropriate to the physical form of the active compound.

In an alternative particular embodiment, the herbicidal composition of the invention comprises an agrochemically acceptable adjuvant comprising 1,2-cyclohexane dicarboxylic acid di-isononyl ester (e.g. CAS Registry no. 166412-78-8), e.g. as available from BASF as Hexamoll™ DINCH™. “Isononyl” in this context is thought to mean one or more, preferably a mixture of two or more, branched isomers of C₉H₁₉. In one particular embodiment, the herbicidal composition, e.g. after dilution (e.g. with water and/or in a spray tank), contains 1,2-cyclohexane dicarboxylic acid di-isononyl ester, and additionally ammonium sulphate and/or isopropyl alcohol.

In an alternative particular embodiment, the herbicidal composition of the invention comprises an agrochemically acceptable adjuvant comprising an organic phosphate and/or organic phosphonate adjuvant. Preferably, the phosphate adjuvant is a tris-[C₄-C₁₂alkyl or 2-(C₂-C₆alkoxy)ethyl-] ester of phosphoric acid, or more preferably is tris-(2-ethylhexyl) phosphate, tris-n-octyl phosphate and/or tris-[2-(n-butoxy)ethyl]phosphate, or most preferably is tris-(2-ethylhexyl) phosphate. Preferably, the phosphonate adjuvant is a bis-(C₃-C₁₂alkyl) ester of a C₃-C₁₂alkyl-phosphonic acid, or more preferably is bis-(2-ethylhexyl) (2-ethylhexyl)phosphonate, bis-(2-ethylhexyl) (n-octyl)phosphonate and/or di-n-butyl (n-butyl)phosphonate.

The formulations (herbicidal compositions) generally contain from 0.1 to 99% by weight, especially from 0.1 to 95% by weight, of a compound of formula I and from 1 to 99.9% by weight of a substantially-inert agrochemically acceptable substance, which preferably includes a formulation adjuvant and/or from 0 to 30% or from 0 to 25% (e.g. from 0.5 to 30% or from 0.5 to 25%) by weight of a surface-active substance. Whereas herbicidal compositions (especially commercial products) will preferably be formulated as concentrates, the end user will normally employ dilute formulations (compositions), e.g. formulations (compositions) diluted with water, in particular when applying the herbicidal composition to weeds and/or to crops of useful plants and/or to the locus thereof.

The rate of application of the compounds of formula I may vary within wide limits and depends upon the nature of the soil, the method of application (pre- or post-emergence; seed dressing; application to the seed furrow; no tillage application etc.), the crop plant, the weed or grass to be controlled, the prevailing climatic conditions, and other factors governed by the method of application, the time of application and the target crop. The compounds of formula I according to the invention are generally applied (preferably post-emergence) at a rate of from 1 to 2000 g/ha, preferably from 1 to 1000 g/ha and most preferably at from 1 to 500 g/ha or from 5 to 500 g/ha.

Preferred formulations/compositions have especially the following representative compositions:

(%=percent by weight of the composition):

### Emulsifiable Concentrates:

active ingredient: 0.3 to 95%, preferably 0.5 to 60% such as 1 to 40%
surface-active agents: 1 to 30%, preferably 3 to 20% such as 5 to 15%
solvents as liquid carrier: 1 to 80%, preferably 1 to 60% such as 1 to 40%

### Dusts:

active ingredient: 0.1 to 10%, preferably 0.1 to 5%
solid carriers: 99.9 to 90%, preferably 99.9 to 99%

### Suspension Concentrates:

active ingredient: 1 to 75%, preferably 3 to 50% or 10 to 50%
water: 98 to 24%, preferably 95 to 30% or 88 to 30%
surface-active agents: 1 to 40%, preferably 2 to 30%

### Wettable Powders:

active ingredient: 0.5 to 90%, preferably 1 to 80%
surface-active agents: 0.5 to 20%, preferably 1 to 15%
solid carriers: 5 to 95%, preferably 15 to 90%

### Granules:

active ingredient: 0.1 to 30%, preferably 0.1 to 15%
solid carriers: 99.5 to 70%, preferably 97 to 85%

### Waterdispersible Granules:

active ingredient: 1 to 90%, preferably 10 to 80%
surface-active agents: 0.5 to 80%, preferably 5 to 30%
solid carriers: 90 to 10%, preferably 70 to 30%

The following Composition (Formulation) Examples further illustrate, but do not limit, the invention.

| F1. Emulsifiable concentrates | a) | b) | c) | d) |
|---|---|---|---|---|
| active ingredient | 5% | 10% | 25% | 50% |
| calcium dodecylbenzene- | 6% | 8% | 6% | 8% |
| sulfonate | | | | |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP (N-methyl-2-pyrrolidone) | — | 10% | — | 20% |
| aromatic hydrocarbon | 85% | 68% | 65% | 16% |
| mixture C₉-C₁₂ | | | | |

Emulsions of any desired concentration can be prepared from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | 40% | 50% | — | — |
| propoxy)-propane | | | | |
| polyethylene glycol MW 400 | 20% | 10% | — | — |
| NMP (N-methyl-2-pyrrolidone) | — | — | 50% | 10% |
| aromatic hydrocarbon | 35% | 30% | — | — |
| mixture C₉-C₁₂ | | | | |

The solutions are suitable for application undiluted or after dilution with water.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly disperse silicic acid | 1% | 3% | 5% | 10% |
| kaolin | 88% | 62% | 35% | — |

The active ingredient is thoroughly mixed with the adjuvants and the mixture is thoroughly ground in a suitable mill, yielding wettable powders which can be diluted with water to give suspensions of any desired concentration.

| F4. Coated granules | a) | b) | c) |
|---|---|---|---|
| active ingredient | 0.1% | 5% | 15% |
| highly dispersed silica | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride, the solution is sprayed onto the carrier and the solvent is subsequently evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silica | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is applied uniformly, in a mixer, to the carrier moistened with polyethylene glycol. Non-dusty coated granules are obtained in this manner.

| F6. Extruded granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants and the mixture is moistened with water. The resulting mixture is extruded and then dried in a stream of air.

| F7. Water-dispersible granules | a) | b) | c) | d) |
|---|---|---|---|---|
| active ingredient | 5% | 10% | 40% | 90% |
| sodium lignosulfonate | 20% | 20% | 15% | 7% |
| dibutyl naphthalene sulfonate | 5% | 5% | 4% | 2% |
| Gum arabic | 2% | 1% | 1% | 1% |
| Diatomaceous earth | 20% | 30% | 5% | — |
| Sodium sulfate | — | 4% | 5% | — |
| kaolin | 48% | 30% | 30% | — |

The active ingredient is mixed and ground with the adjuvants and the mixture is moistened with water. The resulting mixture is extruded and then dried in a stream of air.

| F8. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F9. Suspension concentrates | a) | b) | c) | d) |
|---|---|---|---|---|
| active ingredient | 3% | 10% | 25% | 50% |
| propylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 7% | 6% |
| heteropolysacharide (Xanthan) | 0.2% | 0.2% | 0.2% | 0.2% |
| 1,2-benzisothiazolin-3-one | 0.1% | 0.1% | 0.1% | 0.1% |
| silicone oil emulsion | 0.7% | 0.7% | 0.7% | 0.7% |
| water | 88% | 80% | 60% | 38% |

The finely ground active ingredient is intimately mixed with the adjuvants, yielding a suspension concentrate from which suspensions of any desired concentration can be prepared by dilution with water.

### Herbicidal Uses—Crops of Useful Plants, Weeds, Application Rates, et al.

In a further aspect, the present invention provides a method of controlling weeds (preferably monocotyledonous such as more preferably grassy monocotyledonous weeds) in crops of useful plants, which comprises applying a compound of the formula (I), or a herbicidal composition comprising such a compound, to the weeds and/or to the plants and/or to the locus thereof. Preferably, in this further aspect, the herbicidal composition can be as described hereinabove or hereinbelow, e.g. as described in the “Herbicidal compositions”, “Herbicidal uses”, “Combinations and mixtures” and/or Claims sections hereinabove or hereinbelow.)

In a further aspect, the present invention provides a herbicidal composition, in particular for use in a method of controlling weeds (preferably monocotyledonous weeds such as more preferably grassy monocotyledonous weeds) in crops of useful plants, comprising a compound of formula (I) as defined herein (e.g. a herbicidally effective amount thereof), and an agrochemically acceptable carrier, diluent and/or solvent.

In all aspects of the invention, the compound of the formula (I) is optionally present (e.g. where chemically possible) as an agrochemically acceptable salt thereof (e.g. agrochemically acceptable metal, sulfonium or ammonium salt).

In one embodiment, the herbicidal composition also comprises one or more further herbicides, e.g. as mixture partner(s) for the compound of formula (I), and/or a safener. See the combinations and mixtures section herein for more details of examples of these.

In all aspects of the invention (e.g. the methods of use of the invention), crops of useful plants, e.g. on or in which or at the locus of which the compounds or compositions according to the invention can be used, comprise (e.g. are), in particular: cereals (e.g. non-oat cereals, in particular non-oat non-sorghum non-millet cereals, more particularly wheat, barley, rye and/or triticale), rice, corn (maize), sugarcane, leguminous crops [preferably soybean, peanut, and/or pulse crops; more preferably soybean; wherein typically the pulse crops comprise dry beans (e.g. kidney or haricot or pinto bean which is Phaseolus vulgaris, or mung bean which is Vigna radiata), chickpea, blackeye bean (i.e. black-eyed pea, Vigna unguiculata), lentil, dry broad beans, and/or dry peas such as garden peas], cotton, rape (in particular oilseed rape or canola), sunflower, linseed, sugarbeet, fodder beet, potato, vegetables (preferably dicotyledonous vegetables), flax, tobacco, plantation crops (such as conifer trees, olives and/or olive trees, oil palms, coffee, or vines), and/or fruit crops (in particular dicotyledonous and/or broadleaved fruit, and/or in particular pome fruit, stone fruit, bush fruit, citrus fruit, pineapple, banana, and/or strawberry); and/or turf and/or pastureland grass.

Preferably, in all aspects of the invention, the crops of useful plants, e.g. on or in which or at the locus of which the compounds or compositions according to the invention can be used, comprise (e.g. are): cereals (in particular non-oat cereals, more particularly non-oat non-sorghum non-millet cereals, even more particularly wheat, barley, rye and/or triticale), rice, corn (maize), sugarcane, leguminous crops [preferably soybean, peanut, and/or pulse crops (more preferably soybean)], cotton, rape (in particular oilseed rape or canola), sunflower, linseed, sugarbeet, fodder beet, potato, and/or vegetables (preferably dicotyledonous vegetables).

More preferably, in all aspects of the invention, the crops of useful plants, e.g. on or in which or at the locus of which the compounds or compositions according to the invention can be used, comprise (e.g. are): wheat (e.g. winter wheat, spring wheat, or durum wheat), barley (e.g. winter or spring barley), rye, triticale, sugarcane, leguminous crops [preferably soybean, peanut, and/or pulse crops (more preferably soybean)], cotton, rape (in particular oilseed rape or canola), sunflower, linseed, sugarbeet, fodder beet, potato, and/or vegetables (preferably dicotyledonous vegetables).

Even more preferably, in all aspects of the invention, the crops of useful plants, e.g. on or in which or at the locus of which the compounds or compositions according to the invention can be used, comprise (e.g. are): leguminous crops [preferably soybean, peanut, and/or pulse crops; more preferably soybean; wherein typically the pulse crops comprise dry beans (e.g. kidney or haricot or pinto bean which is Phaseolus vulgaris, or mung bean which is Vigna radiata), chickpea, blackeye bean (i.e. black-eyed pea, Vigna unguiculata), lentil, dry broad beans, and/or dry peas such as garden peas], cotton, rape (in particular oilseed rape or canola), sunflower, sugarbeet, fodder beet, potato, and/or vegetables (preferably dicotyledonous vegetables).

In an alternative more preferable embodiment, in all aspects of the invention, the crops of useful plants, e.g. on or in which or at the locus of which the compounds or compositions according to the invention can be used, comprise (e.g. are): cereals, even more preferably non-oat cereals, yet more preferably wheat (in particular summer or winter wheat, or durum wheat), barley (in particular summer or winter barley), rye and/or triticale.

The term “crops” is to be understood as also including crops that have been rendered tolerant to herbicides or classes of herbicides (for example ALS, GS, EPSPS, PPO and HPPD inhibitors, and/or 2,4-D or dicamba) as a result of conventional methods of breeding or genetic engineering. Examples of crops that have been rendered tolerant e.g. to imidazolinones (which are ALS inhibitors), such as imazamox, by conventional methods of breeding include Clearfield® summer rape (canola) and/or Clearfield® wheat and/or Clearfield® rice (all from BASF). Examples of crops that have been rendered tolerant to herbicides by genetic engineering methods include e.g. glyphosate-resistant or glufosinate-resistant maize or soybean varieties, in particular those commercially available under the trade name RoundupReady® or RoundupReady® 2 (both from Monsanto, both glyphosate-resistant) or LibertyLink® (from Bayer, glufosinate-resistant). Glufosinate-resistant rice (LibertyLink®) also has been published.

Other crops of useful plants include 2,4-D-tolerant soybean, e.g. soybean genetically-modified to be tolerant to the herbicide 2,4-D, or dicamba-tolerant soybean, e.g. soybean genetically-modified to be tolerant to the herbicide dicamba. Such 2,4-D-tolerant or dicamba-tolerant soybean crops can also, in particular, be tolerant to glyphosate or glufosinate. For example, crops of useful plants include soybeans containing a dicamba-tolerance trait combined (stacked) with a glyphosate-tolerance trait, such that these soybeans have tolerance to the herbicides glyphosate and dicamba (for example Genuity® Roundup Ready® 2 Xtend soybeans, currently under development by Monsanto).

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt-176 maize hybrids of NK® (Syngenta Seeds). The Bt toxin is a protein that is formed naturally by Bacillus thuringiensis soil bacteria. Examples of toxins and transgenic plants able to synthesise such toxins are described in EP-A-451 878, EP-A-374 753, WO 93/07278, WO 95/34656, WO 03/052073 and EP-A-427 529. Examples of transgenic plants that contain one or more genes which code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops and their seed material can be resistant to herbicides and at the same time also to insect feeding (“stacked” transgenic events). Seed can, for example, have the ability to express an insecticidally active Cry3 protein and at the same time be glyphosate-tolerant. The term “crops” is to be understood as also including crops obtained as a result of conventional methods of breeding or genetic engineering which contain so-called output traits (e.g. improved flavour, storage stability, nutritional content).

In all aspects of the invention, the weeds, e.g. to be controlled and/or growth-inhibited, may be either monocotyledonous (e.g. grassy) and/or dicotyledonous weeds. Preferably the weeds, e.g. to be controlled and/or growth-inhibited, comprise or are monocotyledonous weeds, more preferably grassy monocotyledonous weeds.

In all aspects of the invention, typically, the monocotyledonous (preferably grassy) weeds, e.g. to be controlled and/or growth-inhibited, comprise (e.g. are) weeds from the genus Agrostis, Alopecurus, Apera, Avena, Brachiaria, Bromus, Cenchrus, Cyperus (a genus of sedges), Digitaria, Echinochloa, Eleusine, Eriochloa, Fimbristylis (a genus of sedges), Juncus (a genus of rushes), Leptochloa, Lolium, Monochoria, Ottochloa, Panicum, Pennisetum, Phalaris, Poa, Rottboellia, Sagittaria, Scirpus (a genus of sedges), Setaria and/or Sorghum; in particular: Alopecurus myosuroides (ALOMY, English name “blackgrass”), Apera spica-venti, Avena fatua (AVEFA, English name “wild oats”), Avena ludoviciana, Avena sterilis, Avena sativa (English name “oats” (volunteer)), Brachiaria decumbens, Brachiaria plantaginea, Bromus tectorum, Digitaria horizontalis, Digitaria insularis, Digitaria sanguinalis (DIGSA), Echinochloa crus-galli (English name “common barnyard grass”, ECHCG), Echinochloa oryzoides, Echinochloa colona or colonum, Eleusine indica, Eriochloa villosa (English name “woolly cupgrass”), Leptochloa chinensis, Leptochloa panicoides, Lolium perenne (LOLPE, English name “perennial ryegrass”), Lolium multiflorum (LOLMU, English name “Italian ryegrass”), Lolium persicum (English name “Persian darnel”), Lolium rigidum, Panicum miliaceum (English name “wild proso millet”), Phalaris minor, Phalaris paradoxa, Poa annua (POAAN, English name “annual bluegrass”), Scirpus maritimus, Scirpus juncoides, Setaria viridis (SETVI, English name “green foxtail”), Setaria faberi (SETFA, English name “giant foxtail”), Setaria glauca, Setaria lutescens (English name “yellow foxtail”), Sorghum bicolor, and/or Sorghum halepense (English name “Johnson grass”); and/or in particular: Brachiaria platyphylla (BRAPP), Panicum dichotomiflorum (PANDI), and/or Sorghum vulgare. Alternatively or additionally, the monocotyledonous (preferably grassy) weeds, e.g. to be controlled and/or growth-inhibited, comprise volunteer corn (volunteer maize) weeds.

In one preferred embodiment of all aspects of the invention, the monocotyledonous weeds, e.g. to be controlled and/or growth-inhibited, are grassy monocotyledonous weeds; in which case they typically comprise (e.g. are) weeds from the genus Agrostis, Alopecurus, Apera, Avena, Brachiaria, Bromus, Cenchrus, Digitaria, Echinochloa, Eleusine, Eriochloa, Leptochloa, Lolium, Ottochloa, Panicum, Pennisetum, Phalaris, Poa, Rottboellia, Setaria and/or Sorghum. Alternatively or additionally, the monocotyledonous (preferably grassy) weeds, e.g. to be controlled and/or growth-inhibited, comprise volunteer corn (volunteer maize) weeds.

In one particular embodiment of all aspects of the invention, the grassy monocotyledonous weeds, e.g. to be controlled and/or growth-inhibited, are “warm-season” (warm climate) grassy weeds; in which case they preferably comprise (e.g. are) weeds from the genus Brachiaria, Cenchrus, Digitaria, Echinochloa, Eleusine, Eriochloa, Leptochloa, Ottochloa, Panicum, Pennisetum, Phalaris, Rottboellia, Setaria and/or Sorghum. Alternatively or additionally, the grassy monocotyledonous weeds, e.g. to be controlled and/or growth-inhibited, comprise volunteer corn (volunteer maize) weeds. More particularly, the grassy monocotyledonous weeds, e.g. to be controlled and/or growth-inhibited, are “warm-season” (warm climate) grassy weeds comprising (e.g. being) weeds from the genus Brachiaria, Cenchrus, Digitaria, Echinochloa, Eleusine, Eriochloa, Panicum, Setaria and/or Sorghum; and/or the grassy monocotyledonous weeds, e.g. to be controlled and/or growth-inhibited, comprise volunteer corn (volunteer maize) weeds.

In another particular embodiment of all aspects of the invention, the grassy monocotyledonous weeds, e.g. to be controlled and/or growth-inhibited, are “cool-season” (cool climate) grassy weeds; in which case they preferably comprise (e.g. are) weeds from the genus Agrostis, Alopecurus, Apera, Avena, Bromus, Lolium and/or Poa.

In non-oat cereal crops such as wheat and/or barley, control and/or growth inhibition of weeds from the genus Alopecurus, Apera, Avena, especially Avena fatua, Bromus, Lolium, Phalaris, and/or Setaria is preferred; in particular Alopecurus, Avena (especially Avena fatua), Lolium and/or Setaria (especially Setaria viridis, Setaria lutescens, Setaria faberi and/or Setaria glauca).

In all aspects of the invention, in a particular embodiment, the weeds, e.g. to be controlled and/or growth-inhibited e.g. by applying a compound of formula (I), may be grassy monocotyledonous weeds (e.g. Agrostis, Alopecurus, Apera, Avena, Brachiaria, Bromus, Cenchrus, Digitaria, Echinochloa, Eleusine, Eriochloa, Leptochloa, Lolium, Ottochloa, Panicum, Pennisetum, Phalaris, Poa, Rottboellia, Setaria and/or Sorghum weeds; more particularly Alopecurus, Apera, Avena, Brachiaria, Bromus, Digitaria, Echinochloa, Eriochloa, Lolium, Panicum, Phalaris, Poa, Setaria and/or Sorghum weeds),
which are resistant to one or more ACCase inhibitor herbicides (ACCase=acetyl-coenzyme A carboxylase) selected from the group consisting of pinoxaden, clodinafop-propargyl, fenoxaprop-P-ethyl, diclofop-methyl, fluazifop-P-butyl, haloxyfop-P-methyl, quizalofop-P-ethyl, propaquizafop, cyhalofop-butyl, clethodim, sethoxydim, cycloxydim, tralkoxydim and butroxydim;
and/or which are resistant to glyphosate;
and/or which are resistant to one or more ALS inhibitor herbicides (ALS=acetolactate synthase), such as one or more sulfonyl urea herbicides (e.g. iodosulfuron-methyl, mesosulfuron-methyl, tribenuron-methyl, triasulfuron, prosulfuron, sulfosulfuron, pyrazosulfuron-ethyl, bensulfuron-methyl, nicosulfuron, flazasulfuron, iofensulfuron, metsulfuron-methyl, or any other sulfonyl urea herbicide disclosed in The Pesticide Manual, 15th edition, (2009) or 16th Edition (2012), ed. C. D. S. Tomlin, British Crop Protection Council) and/or one or more triazolopyrimidine herbicides (e.g. florasulam, pyroxsulam or penoxsulam) and/or one or more pyrimidinyl-(thio or oxy)-benzoate herbicides (e.g. bispyribac-sodium or pyriftalid) and/or one or more sulfonylamino-carbonyl-triazolinone herbicides (e.g. thiencarbazone-methyl, propoxycarbazone-sodium or flucarbazone-sodium) and/or one or more imidazolinone herbicides (e.g. imazamox).

Such resistant (in particular ACCase-inhibitor-resistant, glyphosate-resistant, and/or ALS-inhibitor-resistant) grassy weeds can more particularly comprise Alopecurus myosuroides, Apera spica-venti, Avena fatua, Avena sterilis, Brachiaria decumbens, Brachiaria plantaginea, Digitaria horizontalis, Digitaria insularis, Digitaria sanguinalis, Echinochloa colona, Echinochloa crus-gaffi, Eleusine indica, Lolium multiflorum, Lolium rigidum, Lolium perenne, Phalaris minor, Phalaris paradoxa, Setaria viridis, Setaria faberi, Setaria glauca, and/or Sorghum halepense; or can more particularly comprise Alopecurus myosuroides, Apera spica-venti, Avena fatua, Avena sterilis, Digitaria sanguinalis, Echinochloa colona, Echinochloa crus-gaffi, Lolium multiflorum, Lolium rigidum, Lolium perenne, Phalaris minor, Phalaris paradoxa, Setaria viridis, Setaria faberi and/or Sorghum halepense.

In an even more particular embodiment of the invention, the compound of formula (I) can be applied to grassy monocotyledonous weeds (e.g. selected from one of the above-mentioned list(s) of grassy weeds):

(a1)) which are resistant to one or more ACCase inhibitor herbicides (e.g. selected from the above-mentioned list of ACCase inhibitor herbicides) at least partly by means of mutation (e.g. substitution) of one or more amino acids on the ACCase target site in the weed (e.g. see S. B. Powles and Qin Yu, “Evolution in Action: Plants Resistant to Herbicides”, Annu. Rev. Plant Biol., 2010, 61, pp. 317-347, e.g. see pages 325-327 therein in particular Table 3, incorporated herein by reference, for examples of such resistant weeds and/or amino acid substitutions); and/or
(a2) which are resistant to glyphosate at least partly by means of mutation (e.g. substitution) of one or more amino acids on the EPSPS target site in the weed targeted by glyphosate (e.g. see above-mentioned S. B. Powles and Qin Yu article, pp. 327-329); and/or
(a3) which are resistant to one or more ALS inhibitor herbicides (e.g. selected from the above-mentioned list of ALS inhibitor herbicides) at least partly by mutation (e.g. substitution) of one or more amino acids on the ALS target site in the weed (e.g. see S. B. Powles and Qin Yu, “Evolution in Action: Plants Resistant to Herbicides”, Annu. Rev. Plant Biol., 2010, 61, pp. 317-347, e.g. see pages 322-324 therein in particular Table 2, incorporated herein by reference, for examples of such resistant weeds and/or amino acid substitutions); and/or (b) which are resistant to: one or more ACCase inhibitor herbicides (e.g. selected from the above-mentioned list), and/or glyphosate, and/or one or more ALS inhibitor herbicides (e.g. selected from the above-mentioned list); at least partly by metabolic-type herbicidal resistance e.g. at least partly by cytochrome P450-mediated herbicide metabolism (e.g. see S. B. Powles and Qin Yu, “Evolution in Action: Plants Resistant to Herbicides”, Annu. Rev. Plant Biol., 2010, 61, pp. 317-347, e.g. see Table 4 on page 328 therein, incorporated herein by reference, for examples of such resistant weeds).

In one embodiment, dicotyledonous weeds, e.g. to be controlled, comprise (e.g. are) Abutilon, Amaranthus, Chenopodium, Chrysanthemum, Galium, Ipomoea, Kochia, Nasturtium, Polygonum, Sida, Sinapsis, Solanum, Stellaria, Viola, Veronica and/or Xanthium.

Areas under cultivation, and/or the locus (e.g. of weeds and/or of crops of useful plants), are to be understood as including land where the crop plants are already growing as well as land intended for the cultivation of those crop plants.

In all aspects of the invention, the rate of application (typically to the weeds and/or to the crops of useful plants and/or to the locus thereof) of the compound of formula (I) (which optionally may be an agrochemically acceptable salt thereof) is generally from 1 to 2000 g of the compound of formula (I) per hectare (ha) (measured as the salt-free compound, i.e. excluding the weight of any associated salt counterion(s)), in particular from 5 to 1000 g/ha or from 5 to 500 g/ha or from 10 to 500 g/ha, preferably from 10 to 400 g/ha or from 20 to 300 g/ha, of the compound of formula (I) (measured as the salt-free compound, i.e. excluding the weight of any associated salt counterion(s)). In a preferred embodiment, the above rates of application are for post-emergence application of the compound of formula (I) (which optionally may be an agrochemically acceptable salt thereof).

In all aspects of the invention, the compound of formula (I) can be applied (typically to the weeds and/or to the crops of useful plants and/or to the locus thereof) pre- and/or post-emergence, but preferably is applied post-emergence.

Other Possible Uses— e.g. Possible Insecticidal and/or Acaricidal Uses

The main use and purpose of the compounds of formula (I) according to the invention is their herbicidal use. However, at least some of the compounds of formula (I) may have activity against one or more types of pest (in particular pests associated with agriculture and/or food storage). For example, at least some of the compounds of formula (I) may have at least some insecticidal, acaricidal, molluscicidal and/or nematicidal activity.

At least some of the compounds of formula (I) may have activity against (and/or may help to control and/or combat) insect pests, such as one or more of: Coleoptera, Dictyoptera, Diptera, Hemiptera (including Homoptera), Hymenoptera, Isoptera, Lepidoptera, Orthoptera, Siphonaptera and/or Thysanoptera.

At least some of the compounds of formula (I) may have activity against (and/or may help to control and/or combat) acarine pests and/or pests from the order Acarina, such as one or more of: Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and/or Tetranychus spp.

At least some of the compounds of formula (I) may have activity against (and/or may help to control and/or combat) other (i.e. non-insect, non-acarine) invertebrate pests, for example, nematode and/or mollusc pests.

Insects, acarines, nematodes and/or molluscs are hereinafter collectively referred to as pests.

Examples of pest species, on and/or to which the compounds of formula (I) can be tried and/or applied, include one or more of: Myzus spp. such as Myzus persicae (aphid), Aphis spp. such as Aphis gossypii (aphid) or Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta_migratoria (locust), Chortiocetes terminifera (locust), Diabrotica spp. (rootworms), Panonychus ulmi (European red mite), Panonychus citri (citrus red mite), Tetranychus spp. such as Tetranychus urticae (two-spotted spider mite) or Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), of the Kalotermitidae (for example Neotermes spp.), of the Rhinotermitidae (for example Coptotermes formosanus, Reticulitermes flavipes, R. speratu, R. virginicus, R. hesperus, or R. santonensis) or of the Termitidae (for example Globitermes sulphureus), Solenopsis geminata (fire ant), Monomorium pharaonis (pharaoh's ant), Damalinia spp. or Linognathus spp. (biting lice or sucking lice), Meloidogyne spp. (root knot nematodes), Globodera spp. or Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans_(vinegar eelworm), Trichostrongylus spp. (gastro intestinal nematodes) and/or Deroceras reticulatum (slug).

### Combinations and Mixtures

In a further aspect, the present invention provides a herbicidal composition, e.g. for use in a method of controlling weeds (preferably monocotyledonous weeds such as more preferably grassy monocotyledonous weeds) in crops of useful plants, comprising a compound of formula (I) as defined herein (e.g. a herbicidally effective amount thereof), and an agrochemically acceptable carrier, diluent and/or solvent, and also comprising one or more further herbicides, and/or a safener.

In all aspects of the invention, the compound of the formula (I) is optionally present (e.g. where chemically possible) as an agrochemically acceptable salt thereof.

Examples of these mixtures/compositions, comprising one or more further herbicides and/or a safener, follow.

The compounds of formula (I) according to the invention can be used in combination with one or more further herbicides, e.g. as mixture partner(s) for the compound of formula (I). Preferably, in these mixtures (in particular in the specific mixtures disclosed hereinbelow), the compound of the formula (I) is one of those compounds listed in Tables 1 to 47 and/or one of the exemplified compounds (e.g. one of compounds A1 to A95 or P1 to P7) as disclosed herein e.g. hereinbelow.

In particular, the following mixtures of the compound of formula (I) with one or more further herbicides are particularly disclosed:

compound of formula I+acetochlor, compound of formula I+acifluorfen, compound of formula I+acifluorfen-sodium, compound of formula I+aclonifen, compound of formula I+acrolein, compound of formula I+alachlor, compound of formula I+alloxydim, compound of formula I+allyl alcohol, compound of formula I+ametryn, compound of formula I+amicarbazone, compound of formula I+amidosulfuron, compound of formula I+aminopyralid, compound of formula I+amitrole, compound of formula I+ammonium sulfamate, compound of formula I+anilofos, compound of formula I+asulam, compound of formula I+atraton, compound of formula I+atrazine, compound of formula I+azimsulfuron, compound of formula I+BCPC, compound of formula I+beflubutamid, compound of formula I+benazolin, compound of formula I+benfluralin, compound of formula I+benfuresate, compound of formula I+bensulfuron, compound of formula I+bensulfuron-methyl, compound of formula I+bensulide, compound of formula I+bentazone, compound of formula I+benzfendizone, compound of formula I+benzobicyclon, compound of formula I+benzofenap, compound of formula I+bifenox, compound of formula I+bilanafos, compound of formula I+bispyribac, compound of formula I+bispyribac-sodium, compound of formula I+borax, compound of formula I+bromacil, compound of formula I+bromobutide, compound of formula I+bromoxynil, compound of formula I+bromoxynil heptanoate, compound of formula I+bromoxynil octanoate, compound of formula I+bromoxynil heptanoate+bromoxynil octanoate, compound of formula I+butachlor, compound of formula I+butafenacil, compound of formula I+butamifos, compound of formula I+butralin, compound of formula I+butroxydim, compound of formula I+butylate, compound of formula I+cacodylic acid, compound of formula I+calcium chlorate, compound of formula I+cafenstrole, compound of formula I+carbetamide, compound of formula I+carfentrazone, compound of formula I+carfentrazone-ethyl, compound of formula I+CDEA, compound of formula I+CEPC, compound of formula I+chloransulam, compound of formula I+chloransulam-methyl, compound of formula I+chlorflurenol, compound of formula I+chlorflurenol-methyl, compound of formula I+chloridazon, compound of formula I+chlorimuron, compound of formula I+chlorimuron-ethyl, compound of formula I+chloroacetic acid, compound of formula I+chlorotoluron, compound of formula I+chlorpropham, compound of formula I+chlorsulfuron, compound of formula I+chlorthal, compound of formula I+chlorthal-dimethyl, compound of formula I+cinidon-ethyl, compound of formula I+cinmethylin, compound of formula I+cinosulfuron, compound of formula I+cisanilide, compound of formula I+clethodim, compound of formula I+clodinafop, compound of formula I+clodinafop-propargyl, compound of formula I+clomazone, compound of formula I+clomeprop, compound of formula I+clopyralid, compound of formula I+cloransulam, compound of formula I+cloransulam-methyl, compound of formula I+CMA, compound of formula I+4-CPB, compound of formula I+CPMF, compound of formula I+4-CPP, compound of formula I+CPPC, compound of formula I+cresol, compound of formula I+cumyluron, compound of formula I+cyanamide, compound of formula I+cyanazine, compound of formula I+cycloate, compound of formula I+cyclosulfamuron, compound of formula I+cycloxydim, compound of formula I+cyhalofop, compound of formula I+cyhalofop-butyl, compound of formula I+2,4-D, compound of formula I+2,4-D-dimethylammonium, compound of formula I+2,4-D-2-ethylhexyl, compound of formula I+a choline salt of 2,4-D (see e.g. Examples 2 and 3 of W02010/123871A1), compound of formula I+2,4-D+glyphosate, compound of formula I+2,4-D-dimethylammonium+glyphosate, compound of formula I+2,4-D-2-ethylhexyl+glyphosate, compound of formula I+a choline salt of 2,4-D+glyphosate (see e.g. Examples 2 and 3 of W02010/123871A1), compound of formula I+3,4-DA, compound of formula I+daimuron, compound of formula I+dalapon, compound of formula I+dazomet, compound of formula I+2,4-DB, compound of formula I+3,4-DB, compound of formula I+2,4-DEB, compound of formula I+desmedipham, compound of formula I+dicamba, compound of formula I+dicamba-dimethylammonium, compound of formula I+dicamba-potassium, compound of formula I+dicamba-sodium, compound of formula I+dicamba-diglycolamine, compound of formula I+a N,N-bis-[aminopropyl]methylamine salt of dicamba (see e.g. US2012/0184434A1), compound of formula I+dicamba+glyphosate, compound of formula I+dicamba-dimethylammonium+glyphosate, compound of formula I+dicamba-potassium+glyphosate, compound of formula I+dicamba-sodium+glyphosate, compound of formula I+dicamba-diglycolamine+glyphosate, compound of formula I+a N,N-bis-[aminopropyl]methylamine salt of dicamba+glyphosate, compound of formula I+dichlobenil, compound of formula I+ortho-dichlorobenzene, compound of formula I+para-dichlorobenzene, compound of formula I+dichlorprop, compound of formula I+dichlorprop-P, compound of formula I+diclofop, compound of formula I+diclofop-methyl, compound of formula I+diclosulam, compound of formula I+difenzoquat, compound of formula I+difenzoquat metilsulfate, compound of formula I+diflufenican, compound of formula I+diflufenzopyr, compound of formula I+dimefuron, compound of formula I+dimepiperate, compound of formula I+dimethachlor, compound of formula I+dimethametryn, compound of formula I+dimethenamid, compound of formula I+dimethenamid-P, compound of formula I+dimethipin, compound of formula I+dimethylarsinic acid, compound of formula I+dinitramine, compound of formula I+dinoterb, compound of formula I+diphenamid, compound of formula I+diquat, compound of formula I+diquat dibromide, compound of formula I+dithiopyr, compound of formula I+diuron, compound of formula I+DNOC, compound of formula I+3,4-DP, compound of formula I+DSMA, compound of formula I+EBEP, compound of formula I+endothal, compound of formula I+EPTC, compound of formula I+esprocarb, compound of formula I+ethalfluralin, compound of formula I+ethametsulfuron, compound of formula I+ethametsulfuron-methyl, compound of formula I+ethofumesate, compound of formula I+ethoxyfen, compound of formula I+ethoxysulfuron, compound of formula I+etobenzanid, compound of formula (I)+fenoxaprop, compound of formula (I)+fenoxaprop-ethyl, compound of formula I+fenoxaprop-P, compound of formula I+fenoxaprop-P-ethyl, compound of formula I+fenoxasulfone (CAS Reg. No. 639826-16-7), compound of formula I+fentrazamide, compound of formula I+ferrous sulfate, compound of formula I+flamprop-M, compound of formula I+flazasulfuron, compound of formula I+florasulam, compound of formula I+fluazifop, compound of formula I+fluazifop-butyl, compound of formula I+fluazifop-P, compound of formula I+fluazifop-P-butyl, compound of formula I+flucarbazone, compound of formula I+flucarbazone-sodium, compound of formula I+flucetosulfuron, compound of formula I+fluchloralin, compound of formula I+flufenacet, compound of formula I+flufenpyr, compound of formula I+flufenpyr-ethyl, compound of formula I+flumetsulam, compound of formula I+flumiclorac, compound of formula I+flumiclorac-pentyl, compound of formula I+flumioxazin, compound of formula I+fluometuron, compound of formula I+fluoroglycofen, compound of formula I+fluoroglycofen-ethyl, compound of formula I+flupropanate, compound of formula I+flupyrsulfuron, compound of formula I+flupyrsulfuron-methyl-sodium, compound of formula I+flurenol, compound of formula I+fluridone, compound of formula I+flurochloridone, compound of formula I+fluroxypyr, compound of formula I+fluroxypyr-meptyl, compound of formula I+fluroxypyr-butometyl, compound of formula I+flurtamone, compound of formula I+fluthiacet, compound of formula I+fluthiacet-methyl, compound of formula I+fomesafen, compound of formula I+foramsulfuron, compound of formula I+fosamine, compound of formula I+glufosinate, compound of formula I+glufosinate-ammonium, compound of formula I+glufosinate-P, compound of formula I+glyphosate, compound of formula I+glyphosate-diammonium, compound of formula I+glyphosate-isopropylammonium, compound of formula I+glyphosate-potassium, compound of formula I+halosulfuron, compound of formula I+halosulfuron-methyl, compound of formula I+haloxyfop, compound of formula I+haloxyfop-P, compound of formula (I)+haloxyfop-methyl, compound of formula (I)+haloxyfop-P-methyl, compound of formula I+HC-252, compound of formula I+hexazinone, compound of formula I+imazamethabenz, compound of formula I+imazamethabenz-methyl, compound of formula I+imazamox, compound of formula I+imazapic, compound of formula I+imazapyr, compound of formula I+imazaquin, compound of formula I+imazethapyr, compound of formula I+imazosulfuron, compound of formula I+indanofan, compound of formula I+iodomethane, compound of formula I+iodosulfuron, compound of formula I+iodosulfuron-methyl-sodium, compound of formula I+ioxynil, compound of formula I+ipfencarbazone (CAS Reg. No. 212201-70-2), compound of formula I+isoproturon, compound of formula I+isouron, compound of formula I+isoxaben, compound of formula I+isoxachlortole, compound of formula I+isoxaflutole, compound of formula I+karbutilate, compound of formula I+lactofen, compound of formula I+lenacil, compound of formula I+linuron, compound of formula I+MAA, compound of formula I+MAMA, compound of formula I+MCPA, compound of formula I+MCPA-thioethyl, compound of formula I+MCPB, compound of formula I+mecoprop, compound of formula I+mecoprop-P, compound of formula I+mefenacet, compound of formula I+mefluidide, compound of formula I+mesosulfuron, compound of formula I+mesosulfuron-methyl, compound of formula I+mesotrione, compound of formula I+metam, compound of formula I+metamifop, compound of formula I+metamitron, compound of formula I+metazachlor, compound of formula I+metazosulfuron (NC-620, CAS Reg. No. 868680-84-6), compound of formula I+methabenzthiazuron, compound of formula I+methylarsonic acid, compound of formula I+methyldymron, compound of formula I+methyl isothiocyanate, compound of formula I+metobenzuron, compound of formula I+metolachlor, compound of formula I+S-metolachlor, compound of formula I+metosulam, compound of formula I+metoxuron, compound of formula I+metribuzin, compound of formula I+metsulfuron, compound of formula I+metsulfuron-methyl, compound of formula I+MK-616, compound of formula I+molinate, compound of formula I+monolinuron, compound of formula I+MSMA, compound of formula I+naproanilide, compound of formula I+napropamide, compound of formula I+naptalam, compound of formula I+neburon, compound of formula I+nicosulfuron, compound of formula I+nonanoic acid, compound of formula I+norflurazon, compound of formula I+oleic acid (fatty acids), compound of formula I+orbencarb, compound of formula I+orthosulfamuron, compound of formula I+oryzalin, compound of formula I+oxadiargyl, compound of formula I+oxadiazon, compound of formula I+oxasulfuron, compound of formula I+oxaziclomefone, compound of formula I+oxyfluorfen, compound of formula I+paraquat, compound of formula I+paraquat dichloride, compound of formula I+pebulate, compound of formula I+pendimethalin, compound of formula I+penoxsulam, compound of formula I+pentachlorophenol, compound of formula I+pentanochlor, compound of formula I+pentoxazone, compound of formula I+pethoxamid, compound of formula I+petrolium oils, compound of formula I+phenmedipham, compound of formula I+phenmedipham-ethyl, compound of formula I+picloram, compound of formula I+picolinafen, compound of formula I+pinoxaden, compound of formula I+piperophos, compound of formula I+potassium arsenite, compound of formula I+potassium azide, compound of formula I+pretilachlor, compound of formula I+primisulfuron, compound of formula I+primisulfuron-methyl, compound of formula I+prodiamine, compound of formula I+profluazol, compound of formula I+profoxydim, compound of formula I+prometon, compound of formula I+prometryn, compound of formula I+propachlor, compound of formula I+propanil, compound of formula I+propaquizafop, compound of formula I+propazine, compound of formula I+propham, compound of formula I+propisochlor, compound of formula I+propoxycarbazone, compound of formula I+propoxycarbazone-sodium, compound of formula I+propyrisulfuron (TH-547, CAS Reg. No. 570415-88-2), compound of formula I+propyzamide, compound of formula I+prosulfocarb, compound of formula I+prosulfuron, compound of formula I+pyraclonil, compound of formula I+pyraflufen, compound of formula I+pyraflufen-ethyl, compound of formula I+pyrazolynate, compound of formula I+pyrazosulfuron, compound of formula I+pyrazosulfuron-ethyl, compound of formula I+pyrazoxyfen, compound of formula I+pyribenzoxim, compound of formula I+pyributicarb, compound of formula I+pyridafol, compound of formula I+pyridate, compound of formula I+pyriftalid, compound of formula I+pyriminobac, compound of formula I+pyriminobac-methyl, compound of formula I+pyrimisulfan, compound of formula I+pyrithiobac, compound of formula I+pyrithiobac-sodium, compound of formula I+quinclorac, compound of formula I+quinmerac, compound of formula I+quinoclamine, compound of formula I+quizalofop, compound of formula I+quizalofop-ethyl, compound of formula I+quizalofop-P, compound of formula I+quizalofop-P-ethyl, compound of formula I+quizalofop-P-tefuryl, compound of formula I+rimsulfuron, compound of formula I+sethoxydim, compound of formula I+siduron, compound of formula I+simazine, compound of formula I+simetryn, compound of formula I+SMA, compound of formula I+sodium arsenite, compound of formula I+sodium azide, compound of formula I+sodium chlorate, compound of formula I+sulcotrione, compound of formula I+sulfentrazone, compound of formula I+sulfometuron, compound of formula I+sulfometuron-methyl, compound of formula I+sulfosate, compound of formula I+sulfosulfuron, compound of formula I+sulfuric acid, compound of formula I+tar oils, compound of formula I+2,3,6-TBA, compound of formula I+TCA, compound of formula I+TCA-sodium, compound of formula I+tebuthiuron, compound of formula I+tepraloxydim, compound of formula I+terbacil, compound of formula I+terbumeton, compound of formula I+terbuthylazine, compound of formula I+terbutryn, compound of formula I+thenylchlor, compound of formula I+thiazopyr, compound of formula I+thifensulfuron, compound of formula I+thifensulfuron-methyl, compound of formula I+thiobencarb, compound of formula I+tiocarbazil, compound of formula I+topramezone, compound of formula I+tralkoxydim, compound of formula I+tri-allate, compound of formula I+triasulfuron, compound of formula I+triaziflam, compound of formula I+tribenuron, compound of formula I+tribenuron-methyl, compound of formula I+tricamba, compound of formula I+triclopyr, compound of formula I+trietazine, compound of formula I+trifloxysulfuron, compound of formula I+trifloxysulfuron-sodium, compound of formula I+trifluralin, compound of formula I+triflusulfuron, compound of formula I+triflusulfuron-methyl, compound of formula I+trihydroxytriazine, compound of formula I+tritosulfuron, compound of formula I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS Reg. No. 353292-31-6), compound of formula I+4-[(4,5-dihydro-3-methoxy-4-methyl-5-oxo)-1H-1,2,4-triazol-1-ylcarbonylsulfamoyl]-5-methylthiophene-3-carboxylic acid (BAY636), compound of formula I+BAY747 (CAS Reg. No. 335104-84-2), compound of formula I+topramezone (CAS Reg. No. 210631-68-8), compound of formula I+4-hydroxy-3-[[2-[(2-methoxyethoxy)-methyl]-6-(trifluoromethyl)-3-pyridinyl]carbonyl]bicyclo[3.2.1]oct-3-en-2-one (which is bicyclopyrone, CAS Reg. No. 352010-68-5), compound of formula I+4-hydroxy-3-[[2-(3-methoxypropyl)-6-(difluoromethyl)-3-pyridinyl]carbonyl]bicyclo[3.2.1]oct-3-en-2-one, compound of formula (I)+4-(4′-chloro-4-cyclopropyl-2′-fluorobiphenyl-3-yl)-2,2,6,6-tetramethyl-2H-pyran-3,5(4H,6H)-dione (which is the compound of Example P8 disclosed on pages 31-32 and 35-36 of WO 2010/136431 A9 (Syngenta Limited), and which is also compound A-13 disclosed in pages 4, 5, 7 and 11 of WO 2011/073616 A2 (Syngenta Limited), these parts of these publications being incorporated herein by reference), compound of formula (I)+4-(2′,4′-dichloro-4-cyclopropylbiphenyl-3-yl)-2,2,6,6-tetramethyl-2H-pyran-3,5(4H,6H)-dione (which is the compound of Example P9 disclosed on pages 36-37 and 40-41 of WO 2010/136431 A9 (Syngenta Limited), and which is also compound A-12 disclosed in page 10 of WO 2011/073616 A2 (Syngenta Limited), these parts of these publications being incorporated herein by reference), compound of formula (I)+4-(4′-chloro-4-ethyl-2′-fluorobiphenyl-3-yl)-2,2,6,6-tetramethyl-2H-pyran-3,5(4H,6H)-dione (which is compound A-66 disclosed on page 95 of WO 2008/071405 A1 (Syngenta Participations AG and Syngenta Limited), and which is also compound A-4 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-2,2,6,6-tetramethyl-2H-pyran-3,5(4H,6H)-dione (which is compound A-45 disclosed on page 93 of WO 2008/071405 A1 (Syngenta Participations AG and Syngenta Limited), and which is also the compound of Example P10 disclosed on pages 41 and 45 of WO 2010/136431 A9 (Syngenta Limited), and which is also compound A-7 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-5-(methoxycarbonyloxy)-2,2,6,6-tetramethyl-2H-pyran-3(6H)-one (which is compound D-26 disclosed on page 231 of WO 2008/071405 A1 (Syngenta Participations AG and Syngenta Limited), and which is also compound A-9 disclosed on page 8 of WO 2011/073615 A2 (Syngenta Limited), these parts of these publications being incorporated herein by reference), compound of formula (I)+one of the specific herbicidal compounds disclosed in WO 2010/059676 (Dow, e.g. as defined in one of the examples therein and/or e.g. can be plus cloquintocet-mexyl as safener) these parts of which are incorporated herein by reference, compound of formula (I)+one of the specific herbicidal compounds disclosed in WO 2010/059680 (Dow, e.g. as defined in one of the examples therein and/or e.g. can be plus cloquintocet-mexyl or another safener) these parts of which are incorporated herein by reference, and compound of formula (I)+one of the specific herbicidal compounds disclosed in WO 2010/059671 (Dow, e.g. as defined in one of the examples therein and/or e.g. can be plus a safener) these parts of which are incorporated herein by reference, compound of formula I+halauxifen (which is 4-amino-3-chloro-6-(4-chloro-2-fluoro-3-methoxyphenyl)pyridine-2-carboxylic acid, CAS Reg. No. 943832-60-8), compound of formula I+halauxifen-methyl (which is methyl 4-amino-3-chloro-6-(4-chloro-2-fluoro-3-methoxyphenyl)pyridine-2-carboxylate, CAS Reg. No. 943831-98-9), compound of formula I+aminocyclopyrachlor (which is 6-amino-5-chloro-2-cyclopropylpyrimidine-4-carboxylic acid, CAS Reg. No. 858956-08-8), compound of formula I+aminocyclopyrachlor-methyl (which is methyl 6-amino-5-chloro-2-cyclopropylpyrimidine-4-carboxylate, CAS Reg. No. 858954-83-3), compound of formula I+aminocyclopyrachlor-potassium (which is potassium 6-amino-5-chloro-2-cyclopropylpyrimidine-4-carboxylate, CAS Reg. No. 858956-35-1), compound of formula I+saflufenacil (which is N′-{2-chloro-4-fluoro-5-[1,2,3,6-tetrahydro-3-methyl-2,6-dioxo-4-(trifluoromethyl)pyrimidin-1-yl]benzoyl}-N-isopropyl-N-methylsulfamide, CAS Reg. No. 372137-35-4), compound of formula I+iofensulfuron (which is 1-(2-iodophenylsulfonyl)-3-(4-methoxy-6-methyl-1,3,5-triazin-2-yl)urea, CAS Reg. No. 1144097-22-2), compound of formula I+iofensulfuron-sodium (which is sodium N-(2-iodophenylsulfonyl)-N′-(4-methoxy-6-methyl-1,3,5-triazin-2-yl)carbamimidate, CAS Reg. No. 1144097-30-2), compound of formula I+clacyfos (which is dimethyl [(1RS)-1-(2,4-dichlorophenoxyacetoxy)ethyl]phosphonate, also named Ivxiancaolin or Iüxiancaolin, CAS Reg. No. 215655-76-8), compound of formula I+cyclopyrimorate (which is 6-chloro-3-(2-cyclopropyl-6-methylphenoxy)pyridazin-4-yl morpholine-4-carboxylate, CAS Reg. No. 499231-24-2), or compound of formula I+triafamone (which is N-[2-[(4,6-dimethoxy-1,3,5-triazin-2-yl)carbonyl]-6-fluorophenyl]-N-methyl-1,1-difluoromethanesulfonamide, CAS Reg. No. 874195-61-6).

The mixture partners for the compound of formula (I) are optionally in the form of an ester (in particular an agrochemically acceptable ester) or a salt (in particular an agrochemically acceptable salt) thereof (e.g. where chemically possible). The above-mentioned mixture partners for the compound of formula (I), are generally mentioned e.g. in The Pesticide Manual, 15th Edition, (2009), or 16th Edition (2012) ed. C. D. S. Tomlin, British Crop Production Council.

In the present patent specification, “CAS Reg. No.” or “CAS RN” means the Chemical Abstracts Service Registry Number of the stated compound.

For applications in cereals, the following mixtures are preferred: compound of formula I+aclonifen, compound of formula I+amidosulfuron, compound of formula I+aminopyralid, compound of formula I+beflubutamid, compound of formula I+benfluralin, compound of formula I+bifenox, compound of formula I+bromoxynil, compound of formula I+bromoxynil heptanoate, compound of formula I+bromoxynil octanoate, compound of formula I+bromoxynil heptanoate+bromoxynil octanoate, compound of formula I+butafenacil, compound of formula I+carbetamide, compound of formula I+carfentrazone, compound of formula I+carfentrazone-ethyl, compound of formula I+chlorotoluron, compound of formula I+chlorpropham, compound of formula I+chlorsulfuron, compound of formula I+cinidon-ethyl, compound of formula I+clodinafop, compound of formula I+clodinafop-propargyl, compound of formula I+clopyralid, compound of formula I+2,4-D, compound of formula I+2,4-D-dimethylammonium, compound of formula I+2,4-D-2-ethylhexyl, compound of formula I+a choline salt of 2,4-D (see e.g. Examples 2 and 3 of W02010/123871A1), compound of formula I+dicamba, compound of formula I+dicamba-dimethylammonium, compound of formula I+dicamba-potassium, compound of formula I+dicamba-sodium, compound of formula I+dicamba-diglycolamine, compound of formula I+a N,N-bis-[aminopropyl]methylamine salt of dicamba (see e.g. US2012/0184434A1), compound of formula I+dichlobenil, compound of formula I+dichlorprop, compound of formula I+diclofop, compound of formula I+diclofop-methyl, compound of formula I+difenzoquat, compound of formula I+difenzoquat metilsulfate, compound of formula I+diflufenican, compound of formula I+diquat, compound of formula I+diquat dibromide, compound of formula (I)+fenoxaprop, compound of formula (I)+fenoxaprop-ethyl, compound of formula I+fenoxaprop-P, compound of formula I+fenoxaprop-P-ethyl, compound of formula I+flamprop-M, compound of formula I+florasulam, compound of formula I+fluazifop-P-butyl, compound of formula I+flucarbazone, compound of formula I+flucarbazone-sodium, compound of formula I+flufenacet, compound of formula I+flupyrsulfuron, compound of formula I+flupyrsulfuron-methyl-sodium, compound of formula I+flurochloridone, compound of formula I+fluroxypyr, compound of formula I+fluroxypyr-meptyl, compound of formula I+fluroxypyr-butometyl, compound of formula I+flurtamone, compound of formula I+imazamethabenz-methyl, compound of formula I+imazamox, compound of formula I+iodosulfuron, compound of formula I+iodosulfuron-methyl-sodium, compound of formula I+ioxynil, compound of formula I+isoproturon, compound of formula I+linuron, compound of formula I+MCPA, compound of formula I+mecoprop, compound of formula I+mecoprop-P, compound of formula I+mesosulfuron, compound of formula I+mesosulfuron-methyl, compound of formula I+mesotrione, compound of formula I+metribuzin, compound of formula I+metsulfuron, compound of formula I+metsulfuron-methyl, compound of formula I+pendimethalin, compound of formula I+picolinafen, compound of formula I+pinoxaden, compound of formula I+prodiamine, compound of formula I+propanil, compound of formula I+propoxycarbazone, compound of formula I+propoxycarbazone-sodium, compound of formula I+prosulfocarb, compound of formula I+pyrasulfotole, compound of formula I+pyridate, compound of formula I+pyroxasulfone (KIH-485), compound of formula I+pyroxsulam compound of formula I+sulfosulfuron, compound of formula I+tembotrione, compound of formula I+terbutryn, compound of formula I+thifensulfuron, compound of formula I+thiencarbazone, compound of formula I+thifensulfuron-methyl, compound of formula I+topramezone, compound of formula I+tralkoxydim, compound of formula I+tri-allate, compound of formula I+triasulfuron, compound of formula I+tribenuron, compound of formula I+tribenuron-methyl, compound of formula I+trifluralin, compound of formula I+trinexapac-ethyl and compound of formula I+tritosulfuron, compound of formula I+4-hydroxy-3-[[2-[(2-methoxyethoxy)methyl]-6-(trifluoromethyl)-3-pyridinyl]carbonyl]-bicyclo[3.2.1]oct-3-en-2-one (which is bicyclopyrone, CAS Reg. No. 352010-68-5), compound of formula (I)+one of the specific herbicidal compounds disclosed in WO 2010/059676 (Dow, e.g. as defined in one of the examples therein and/or e.g. can be plus cloquintocet-mexyl as safener) these parts of which are incorporated herein by reference, compound of formula (I)+one of the specific herbicidal compounds disclosed in WO 2010/059680 (Dow, e.g. as defined in one of the examples therein and/or e.g. can be plus cloquintocet-mexyl or another safener) these parts of which are incorporated herein by reference, compound of formula I+halauxifen (which is 4-amino-3-chloro-6-(4-chloro-2-fluoro-3-methoxyphenyl)pyridine-2-carboxylic acid, CAS Reg. No. 943832-60-8), compound of formula I+halauxifen-methyl (which is methyl 4-amino-3-chloro-6-(4-chloro-2-fluoro-3-methoxyphenyl)pyridine-2-carboxylate, CAS Reg. No. 943831-98-9), compound of formula I+iofensulfuron (which is 1-(2-iodophenylsulfonyl)-3-(4-methoxy-6-methyl-1,3,5-triazin-2-yl)urea, CAS Reg. No. 1144097-22-2), or compound of formula I+iofensulfuron-sodium (which is sodium N-(2-iodophenylsulfonyl)-N′-(4-methoxy-6-methyl-1,3,5-triazin-2-yl)carbamimidate, CAS Reg. No. 1144097-30-2);

wherein the mixture partners for the compound of formula (I) may optionally be in the form of an ester (in particular an agrochemically acceptable ester) or a salt (in particular an agrochemically acceptable salt) thereof (e.g. where chemically possible).

For applications in cereals, more preferred is a mixture comprising: a compound of formula (I)+amidosulfuron, compound of formula (I)+aminopyralid, compound of formula (I)+beflubutamid, compound of formula (I)+bromoxynil, compound of formula (I)+bromoxynil heptanoate, compound of formula (I)+bromoxynil octanoate, compound of formula (I)+bromoxynil heptanoate+bromoxynil octanoate, compound of formula (I)+carfentrazone, compound of formula (I)+carfentrazone-ethyl, compound of formula (I)+chlorotoluron, compound of formula (I)+chlorsulfuron, compound of formula (I)+clodinafop, compound of formula (I)+clodinafop-propargyl, compound of formula (I)+clopyralid, compound of formula (I)+2,4-D, compound of formula (I)+2,4-D-dimethylammonium, compound of formula (I)+2,4-D-2-ethylhexyl, compound of formula (I)+a choline salt of 2,4-D (see e.g. Examples 2 and 3 of W02010/123871A1), compound of formula (I)+dicamba, compound of formula (I)+dicamba-dimethylammonium, compound of formula (I)+dicamba-potassium, compound of formula (I)+dicamba-sodium, compound of formula (I)+dicamba-diglycolamine, compound of formula (I)+a N,N-bis-[aminopropyl]methylamine salt of dicamba (see e.g. US2012/0184434A1), compound of formula (I)+difenzoquat, compound of formula (I)+difenzoquat metilsulfate, compound of formula (I)+diflufenican, compound of formula (I)+fenoxaprop-P, compound of formula (I)+fenoxaprop-P-ethyl, compound of formula (I)+florasulam, compound of formula (I)+flucarbazone, compound of formula (I)+flucarbazone-sodium, compound of formula (I)+flufenacet, compound of formula (I)+flupyrsulfuron, compound of formula (I)+flupyrsulfuron-methyl-sodium, compound of formula (I)+fluroxypyr, compound of formula I+fluroxypyr-meptyl, compound of formula I+fluroxypyr-butometyl, compound of formula (I)+flurtamone, compound of formula (I)+iodosulfuron, compound of formula (I)+iodosulfuron-methyl-sodium, compound of formula (I)+MCPA, compound of formula (I)+mesosulfuron, compound of formula (I)+mesosulfuron-methyl, compound of formula (I)+metsulfuron, compound of formula (I)+metsulfuron-methyl, compound of formula (I)+pendimethalin, compound of formula (I)+picolinafen, compound of formula (I)+pinoxaden, compound of formula (I)+prosulfocarb, compound of formula (I)+pyrasulfotole, compound of formula (I)+pyroxasulfone (KIH-485), compound of formula (I)+pyroxsulam, compound of formula (I)+sulfosulfuron, compound of formula (I)+thifensulfuron, compound of formula (I)+thifensulfuron-methyl, compound of formula I+topramezone, compound of formula (I)+tralkoxydim, compound of formula (I)+triasulfuron, compound of formula (I)+tribenuron, compound of formula (I)+tribenuron-methyl, compound of formula (I)+trifluralin, compound of formula (I)+trinexapac-ethyl, compound of formula (I)+tritosulfuron, compound of formula I+4-hydroxy-3-[[2-[(2-methoxyethoxy)-methyl]-6-(trifluoromethyl)-3-pyridinyl]carbonyl]-bicyclo[3.2.1]oct-3-en-2-one (which is bicyclopyrone, CAS Reg. No. 352010-68-5), compound of formula (I)+one of the specific herbicidal compounds disclosed in WO 2010/059676 (Dow, e.g. as defined in one of the examples therein and/or e.g. can be plus cloquintocet-mexyl as safener) these parts of which are incorporated herein by reference, compound of formula (I)+one of the specific herbicidal compounds disclosed in WO 2010/059680 (Dow, e.g. as defined in one of the examples therein and/or e.g. can be plus cloquintocet-mexyl or another safener) these parts of which are incorporated herein by reference, compound of formula I+halauxifen (which is 4-amino-3-chloro-6-(4-chloro-2-fluoro-3-methoxyphenyl)pyridine-2-carboxylic acid, CAS Reg. No. 943832-60-8), compound of formula I+halauxifen-methyl (which is methyl 4-amino-3-chloro-6-(4-chloro-2-fluoro-3-methoxyphenyl)pyridine-2-carboxylate, CAS Reg. No. 943831-98-9), compound of formula I+iofensulfuron (which is 1-(2-iodophenylsulfonyl)-3-(4-methoxy-6-methyl-1,3,5-triazin-2-yl)urea, CAS Reg. No. 1144097-22-2), or compound of formula I+iofensulfuron-sodium (which is sodium N-(2-iodophenylsulfonyl)-N′-(4-methoxy-6-methyl-1,3,5-triazin-2-yl)carbamimidate, CAS Reg. No. 1144097-30-2);

wherein the mixture partners for the compound of formula (I) may optionally be in the form of an ester (in particular an agrochemically acceptable ester) or a salt (in particular an agrochemically acceptable salt) thereof (e.g. where chemically possible).

For applications in rice, the following mixtures are preferred: compound of formula (I)+azimsulfuron, compound of formula (I)+bensulfuron, compound of formula (I)+bensulfuron-methyl, compound of formula (I)+benzobicyclon, compound of formula (I)+benzofenap, compound of formula (I)+bispyribac, compound of formula (I)+bispyribac-sodium, compound of formula (I)+butachlor, compound of formula (I)+cafenstrole, compound of formula (I)+cinosulfuron, compound of formula (I)+clomazone, compound of formula (I)+clomeprop, compound of formula (I)+cyclosulfamuron, compound of formula (I)+cyhalofop, compound of formula (I)+cyhalofop-butyl, compound of formula (I)+2,4-D, compound of formula (I)+2,4-D-dimethylammonium, compound of formula (I)+2,4-D-2-ethylhexyl, compound of formula (I)+a choline salt of 2,4-D (see e.g. Examples 2 and 3 of W02010/123871A1), compound of formula (I)+daimuron, compound of formula (I)+dicamba, compound of formula (I)+dicamba-dimethylammonium, compound of formula (I)+dicamba-potassium, compound of formula (I)+dicamba-sodium, compound of formula (I)+dicamba-diglycolamine, compound of formula (I)+a N,N-bis-[aminopropyl]methylamine salt of dicamba (see e.g. US2012/0184434A1), compound of formula (I)+diquat, compound of formula (I)+diquat dibromide, compound of formula (I)+esprocarb, compound of formula (I)+ethoxysulfuron, compound of formula (I)+fenoxaprop, compound of formula (I)+fenoxaprop-ethyl, compound of formula (I)+fenoxaprop-P, compound of formula (I)+fenoxaprop-P-ethyl, compound of formula I+fenoxasulfone (CAS Reg. No. 639826-16-7), compound of formula (I)+fentrazamide, compound of formula (I)+florasulam, compound of formula (I)+glufosinate-ammonium, compound of formula (I)+glyphosate, compound of formula (I)+glyphosate-diammonium, compound of formula (I)+glyphosate-isopropylammonium, compound of formula (I)+glyphosate-potassium, compound of formula (I)+halosulfuron, compound of formula (I)+halosulfuron-methyl, compound of formula (I)+imazosulfuron, compound of formula I+ipfencarbazone (CAS Reg. No. 212201-70-2), compound of formula (I)+MCPA, compound of formula (I)+mefenacet, compound of formula (I)+mesotrione, compound of formula (I)+metamifop, compound of formula I+metazosulfuron (NC-620, CAS Reg. No. 868680-84-6), compound of formula (I)+metsulfuron, compound of formula (I)+metsulfuron-methyl, compound of formula (I)+n-methyl glyphosate, compound of formula (I)+orthosulfamuron, compound of formula (I)+oryzalin, compound of formula (I)+oxadiargyl, compound of formula (I)+oxadiazon, compound of formula (I)+paraquat dichloride, compound of formula (I)+pendimethalin, compound of formula (I)+penoxsulam, compound of formula (I)+pretilachlor, compound of formula (I)+profoxydim, compound of formula (I)+propanil, compound of formula I+propyrisulfuron (TH-547, CAS Reg. No. 570415-88-2), compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyriftalid, compound of formula (I)+pyriminobac, compound of formula (I)+pyriminobac-methyl, compound of formula (I)+pyrimisulfan, compound of formula (I)+quinclorac, compound of formula (I)+tefuryltrione, compound of formula (I)+triasulfuron and compound of formula (I)+trinexapac-ethyl, compound of formula (I)+4-(4′-chloro-4-cyclopropyl-2′-fluorobiphenyl-3-yl)-2,2,6,6-tetramethyl-2H-pyran-3,5(4H,6H)-dione (which is the compound of Example P8 disclosed on pages 31-32 and 35-36 of WO 2010/136431 A9 (Syngenta Limited), and which is also compound A-13 disclosed in pages 4, 5, 7 and 11 of WO 2011/073616 A2 (Syngenta Limited), these parts of these publications being incorporated herein by reference), compound of formula (I)+4-(2′,4′-dichloro-4-cyclopropylbiphenyl-3-yl)-2,2,6,6-tetramethyl-2H-pyran-3,5(4H,6H)-dione (which is the compound of Example P9 disclosed on pages 36-37 and 40-41 of WO 2010/136431 A9 (Syngenta Limited), and which is also compound A-12 disclosed in page 10 of WO 2011/073616 A2 (Syngenta Limited), these parts of these publications being incorporated herein by reference), compound of formula (I)+4-(4′-chloro-4-ethyl-2′-fluorobiphenyl-3-yl)-2,2,6,6-tetramethyl-2H-pyran-3,5(4H,6H)-dione (which is compound A-66 disclosed on page 95 of WO 2008/071405 A1 (Syngenta Participations AG and Syngenta Limited), and which is also compound A-4 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-2,2,6,6-tetramethyl-2H-pyran-3,5(4H,6H)-dione (which is compound A-45 disclosed on page 93 of WO 2008/071405 A1 (Syngenta Participations AG and Syngenta Limited), and which is also the compound of Example P10 disclosed on pages 41 and 45 of WO 2010/136431 A9 (Syngenta Limited), and which is also compound A-7 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-5-(methoxycarbonyloxy)-2,2,6,6-tetramethyl-2H-pyran-3(6H)-one (which is compound D-26 disclosed on page 231 of WO 2008/071405 A1 (Syngenta Participations AG and Syngenta Limited), and which is also compound A-9 disclosed on page 8 of WO 2011/073615 A2 (Syngenta Limited), these parts of these publications being incorporated herein by reference), compound of formula (I)+one of the specific herbicidal compounds disclosed in WO 2010/059671 (Dow, e.g. as defined in one of the examples therein and/or e.g. can be plus a safener) these parts of which are incorporated herein by reference, compound of formula I+halauxifen (which is 4-amino-3-chloro-6-(4-chloro-2-fluoro-3-methoxyphenyl)pyridine-2-carboxylic acid, CAS Reg. No. 943832-60-8), compound of formula I+halauxifen-methyl (which is methyl 4-amino-3-chloro-6-(4-chloro-2-fluoro-3-methoxyphenyl)pyridine-2-carboxylate, CAS Reg. No. 943831-98-9), compound of formula I+iofensulfuron (which is 1-(2-iodophenylsulfonyl)-3-(4-methoxy-6-methyl-1,3,5-triazin-2-yl)urea, CAS Reg. No. 1144097-22-2), compound of formula I+iofensulfuron-sodium (which is sodium N-(2-iodophenylsulfonyl)-N′-(4-methoxy-6-methyl-1,3,5-triazin-2-yl)carbamimidate, CAS Reg. No. 1144097-30-2), or compound of formula I+triafamone (which is N-[2-[(4,6-dimethoxy-1,3,5-triazin-2-yl)carbonyl]-6-fluorophenyl]-N-methyl-1,1-difluoromethanesulfonamide, CAS Reg. No. 874195-61-6);

wherein the mixture partners for the compound of formula (I) may optionally be in the form of an ester (in particular an agrochemically acceptable ester) or a salt (in particular an agrochemically acceptable salt) thereof (e.g. where chemically possible).

For applications in rice, more preferred is a mixture comprising: a compound of formula (I)+azimsulfuron, compound of formula (I)+bensulfuron, compound of formula (I)+bensulfuron-methyl, compound of formula (I)+benzobicyclon, compound of formula (I)+benzofenap, compound of formula (I)+bispyribac, compound of formula (I)+bispyribac-sodium, compound of formula (I)+clomazone, compound of formula (I)+clomeprop, compound of formula (I)+cyhalofop, compound of formula (I)+cyhalofop-butyl, compound of formula (I)+2,4-D, compound of formula (I)+2,4-D-dimethylammonium, compound of formula (I)+2,4-D-2-ethylhexyl, compound of formula (I)+a choline salt of 2,4-D (see e.g. Examples 2 and 3 of W02010/123871A1), compound of formula (I)+daimuron, compound of formula (I)+dicamba, compound of formula (I)+dicamba-dimethylammonium, compound of formula (I)+dicamba-potassium, compound of formula (I)+dicamba-sodium, compound of formula (I)+dicamba-diglycolamine, compound of formula (I)+a N,N-bis-[aminopropyl]methylamine salt of dicamba (see e.g. US2012/0184434A1), compound of formula (I)+esprocarb, compound of formula (I)+ethoxysulfuron, compound of formula (I)+fenoxaprop-P, compound of formula (I)+fenoxaprop-P-ethyl, compound of formula I+fenoxasulfone (CAS Reg. No. 639826-16-7), compound of formula (I)+fentrazamide, compound of formula (I)+florasulam, compound of formula (I)+halosulfuron, compound of formula (I)+halosulfuron-methyl, compound of formula (I)+imazosulfuron, compound of formula I+ipfencarbazone (CAS Reg. No. 212201-70-2), compound of formula (I)+MCPA, compound of formula (I)+mefenacet, compound of formula (I)+mesotrione, compound of formula I+metazosulfuron (NC-620, CAS Reg. No. 868680-84-6), compound of formula (I)+metsulfuron, compound of formula (I)+metsulfuron-methyl, compound of formula (I)+orthosulfamuron, compound of formula (I)+oxadiargyl, compound of formula (I)+oxadiazon, compound of formula (I)+pendimethalin, compound of formula (I)+penoxsulam, compound of formula (I)+pretilachlor, compound of formula I+propyrisulfuron (TH-547, CAS Reg. No. 570415-88-2), compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyriftalid, compound of formula (I)+pyriminobac, compound of formula (I)+pyriminobac-methyl, compound of formula (I)+pyrimisulfan, compound of formula (I)+quinclorac, compound of formula (I)+tefuryltrione, compound of formula (I)+triasulfuron and compound of formula (I)+trinexapac-ethyl, compound of formula (I)+4-(4′-chloro-4-cyclopropyl-2′-fluorobiphenyl-3-yl)-2,2,6,6-tetramethyl-2H-pyran-3,5(4H,6H)-dione (which is the compound of Example P8 disclosed on pages 31-32 and 35-36 of WO 2010/136431 A9 (Syngenta Limited), and which is also compound A-13 disclosed in pages 4, 5, 7 and 11 of WO 2011/073616 A2 (Syngenta Limited), these parts of these publications being incorporated herein by reference), compound of formula (I)+4-(2′,4′-dichloro-4-cyclopropylbiphenyl-3-yl)-2,2,6,6-tetramethyl-2H-pyran-3,5(4H,6H)-dione (which is the compound of Example P9 disclosed on pages 36-37 and 40-41 of WO 2010/136431 A9 (Syngenta Limited), and which is also compound A-12 disclosed in page 10 of WO 2011/073616 A2 (Syngenta Limited), these parts of these publications being incorporated herein by reference), compound of formula (I)+4-(4′-chloro-4-ethyl-2′-fluorobiphenyl-3-yl)-2,2,6,6-tetramethyl-2H-pyran-3,5(4H,6H)-dione (which is compound A-66 disclosed on page 95 of WO 2008/071405 A1 (Syngenta Participations AG and Syngenta Limited), and which is also compound A-4 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-2,2,6,6-tetramethyl-2H-pyran-3,5(4H,6H)-dione (which is compound A-45 disclosed on page 93 of WO 2008/071405 A1 (Syngenta Participations AG and Syngenta Limited), and which is also the compound of Example P10 disclosed on pages 41 and 45 of WO 2010/136431 A9 (Syngenta Limited), and which is also compound A-7 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-5-(methoxycarbonyloxy)-2,2,6,6-tetramethyl-2H-pyran-3(6H)-one (which is compound D-26 disclosed on page 231 of WO 2008/071405 A1 (Syngenta Participations AG and Syngenta Limited), and which is also compound A-9 disclosed on page 8 of WO 2011/073615 A2 (Syngenta Limited), these parts of these publications being incorporated herein by reference), compound of formula (I)+one of the specific herbicidal compounds disclosed in WO 2010/059671 (Dow, e.g. as defined in one of the examples therein and/or e.g. can be plus a safener) these parts of which are incorporated herein by reference, compound of formula I+halauxifen (which is 4-amino-3-chloro-6-(4-chloro-2-fluoro-3-methoxyphenyl)pyridine-2-carboxylic acid, CAS Reg. No. 943832-60-8), compound of formula I+halauxifen-methyl (which is methyl 4-amino-3-chloro-6-(4-chloro-2-fluoro-3-methoxyphenyl)pyridine-2-carboxylate, CAS Reg. No. 943831-98-9), compound of formula I+iofensulfuron (which is 1-(2-iodophenylsulfonyl)-3-(4-methoxy-6-methyl-1,3,5-triazin-2-yl)urea, CAS Reg. No. 1144097-22-2), compound of formula I+iofensulfuron-sodium (which is sodium N-(2-iodophenylsulfonyl)-N′-(4-methoxy-6-methyl-1,3,5-triazin-2-yl)carbamimidate, CAS Reg. No. 1144097-30-2), or compound of formula I+triafamone (which is N-[2-[(4,6-dimethoxy-1,3,5-triazin-2-yl)carbonyl]-6-fluorophenyl]-N-methyl-1,1-difluoromethanesulfonamide, CAS Reg. No. 874195-61-6);

wherein the mixture partners for the compound of formula (I) may optionally be in the form of an ester (in particular an agrochemically acceptable ester) or a salt (in particular an agrochemically acceptable salt) thereof (e.g. where chemically possible).

For applications in soybean, the following mixtures are preferred:

compound of formula (I)+acifluorfen, compound of formula (I)+acifluorfen-sodium, compound of formula (I)+ametryn, compound of formula (I)+atrazine, compound of formula (I)+bentazone, compound of formula (I)+bicyclopyrone, compound of formula (I)+bromoxynil, compound of formula (I)+bromoxynil heptanoate, compound of formula (I)+bromoxynil octanoate, compound of formula (I)+bromoxynil heptanoate+bromoxynil octanoate, compound of formula (I)+carfentrazone, compound of formula (I)+carfentrazone-ethyl, compound of formula (I)+chloransulam, compound of formula (I)+chloransulam-methyl, compound of formula (I)+chlorimuron, compound of formula (I)+chlorimuron-ethyl, compound of formula (I)+clethodim, compound of formula (I)+clomazone, compound of formula (I)+cyanazine, compound of formula (I)+2,4-D (especially for applications to 2,4-D-tolerant soybean, e.g. genetically-modified), compound of formula (I)+2,4-D-dimethylammonium (especially for applications to 2,4-D-tolerant soybean, e.g. genetically-modified), compound of formula (I)+2,4-D-2-ethylhexyl (especially for applications to 2,4-D-tolerant soybean, e.g. genetically-modified), compound of formula (I)+a choline salt of 2,4-D (see e.g. Examples 2 and 3 of W02010/123871A1) (especially for applications to 2,4-D-tolerant soybean, e.g. genetically-modified), compound of formula (I)+2,4-D+glyphosate (especially for applications to 2,4-D-tolerant and/or glyphosate-tolerant soybean, e.g. genetically-modified), compound of formula (I)+2,4-D-dimethylammonium+glyphosate (especially for applications to 2,4-D-tolerant and/or glyphosate-tolerant soybean, e.g. genetically-modified), compound of formula (I)+2,4-D-2-ethylhexyl+glyphosate (especially for applications to 2,4-D-tolerant and/or glyphosate-tolerant soybean, e.g. genetically-modified), compound of formula I+a choline salt of 2,4-D+glyphosate (see e.g. Examples 2 and 3 of W02010/123871A1) (especially for applications to dicamba-tolerant and/or glyphosate-tolerant soybean, e.g. genetically-modified), compound of formula (I)+dicamba (especially for applications to dicamba-tolerant soybean, e.g. genetically-modified), compound of formula (I)+dicamba-dimethylammonium (especially for applications to dicamba-tolerant soybean, e.g. genetically-modified), compound of formula (I)+dicamba-potassium (especially for applications to dicamba-tolerant soybean, e.g. genetically-modified), compound of formula (I)+dicamba-sodium (especially for applications to dicamba-tolerant soybean, e.g. genetically-modified), compound of formula (I)+dicamba-diglycolamine (especially for applications to dicamba-tolerant soybean, e.g. genetically-modified), compound of formula (I)+a N,N-bis-[aminopropyl]methylamine salt of dicamba (see e.g. US2012/0184434A1) (especially for applications to dicamba-tolerant soybean, e.g. genetically-modified), compound of formula (I)+dicamba+glyphosate (especially for applications to dicamba-tolerant and/or glyphosate-tolerant soybean, e.g. genetically-modified), compound of formula (I)+dicamba-dimethylammonium+glyphosate (especially for applications to dicamba-tolerant and/or glyphosate-tolerant soybean, e.g. genetically-modified), compound of formula (I)+dicamba-potassium+glyphosate (especially for applications to dicamba-tolerant and/or glyphosate-tolerant soybean, e.g. genetically-modified), compound of formula (I)+dicamba-sodium+glyphosate (especially for applications to dicamba-tolerant and/or glyphosate-tolerant soybean, e.g. genetically-modified), compound of formula (I)+dicamba-diglycolamine+glyphosate (especially for applications to dicamba-tolerant and/or glyphosate-tolerant soybean, e.g. genetically-modified), compound of formula (I)+a N,N-bis-[aminopropyl]methylamine salt of dicamba+glyphosate (especially for applications to dicamba-tolerant and/or glyphosate-tolerant soybean, e.g. genetically-modified), compound of formula (I)+diclosulam, compound of formula (I)+dimethenamid, compound of formula (I)+dimethenamid-P, compound of formula (I)+diquat, compound of formula (I)+diquat dibromide, compound of formula (I)+diuron, compound of formula (I)+fenoxaprop, compound of formula (I)+fenoxaprop-ethyl, compound of formula (I)+fenoxaprop-P, compound of formula (I)+fenoxaprop-P-ethyl, compound of formula (I)+fluazifop, compound of formula (I)+fluazifop-butyl, compound of formula (I)+fluazifop-P, compound of formula (I)+fluazifop-P-butyl, compound of formula (I)+flufenacet, compound of formula (I)+flumetsulam, compound of formula (I)+flumioxazin, compound of formula (I)+fluthiacet, compound of formula (I)+fluthiacet-methyl, compound of formula (I)+fomesafen, compound of formula (I)+glufosinate, compound of formula (I)+glufosinate-ammonium, compound of formula (I)+glyphosate, compound of formula (I)+glyphosate-diammonium, compound of formula (I)+glyphosate-isopropylammonium, compound of formula (I)+glyphosate-potassium, compound of formula (I)+imazethapyr, compound of formula (I)+lactofen, compound of formula (I)+mesotrione, compound of formula (I)+metolachlor, compound of formula (I)+S-metolachlor, compound of formula (I)+metribuzin, compound of formula (I)+oxyfluorfen, compound of formula (I)+paraquat, compound of formula (I)+paraquat dichloride, compound of formula (I)+pendimethalin, compound of formula (I)+pyroxasulfone, compound of formula I+quizalofop, compound of formula I+quizalofop-ethyl, compound of formula I+quizalofop-P, compound of formula I+quizalofop-P-ethyl, compound of formula I+quizalofop-P-tefuryl, compound of formula (I)+saflufenacil, compound of formula (I)+sethoxydim, compound of formula (I)+sulfentrazone, compound of formula (I)+thifensulfuron, compound of formula (I)+thifensulfuron-methyl, compound of formula (I)+tribenuron, compound of formula (I)+tribenuron-methyl, compound of formula (I)+trifluralin, compound of formula (I)+4-(4′-chloro-4-cyclopropyl-2′-fluorobiphenyl-3-yl)-2,2,6,6-tetramethyl-2H-pyran-3,5(4H,6H)-dione (which is the compound of Example P8 disclosed on pages 31-32 and 35-36 of WO 2010/136431 A9 (Syngenta Limited), and which is also compound A-13 disclosed in pages 4, 5, 7 and 11 of WO 2011/073616 A2 (Syngenta Limited), these parts of these publications being incorporated herein by reference), compound of formula (I)+4-(2′,4′-dichloro-4-cyclopropylbiphenyl-3-yl)-2,2,6,6-tetramethyl-2H-pyran-3,5(4H,6H)-dione (which is the compound of Example P9 disclosed on pages 36-37 and 40-41 of WO 2010/136431 A9 (Syngenta Limited), and which is also compound A-12 disclosed in page 10 of WO 2011/073616 A2 (Syngenta Limited), these parts of these publications being incorporated herein by reference), compound of formula (I)+4-(4′-chloro-4-ethyl-2′-fluorobiphenyl-3-yl)-2,2,6,6-tetramethyl-2H-pyran-3,5(4H,6H)-dione (which is compound A-66 disclosed on page 95 of WO 2008/071405 A1 (Syngenta Participations AG and Syngenta Limited), and which is also compound A-4 disclosed on page 7 of WO 2011/073615 A2 (Syngenta Limited), these parts of these publications being incorporated herein by reference), compound of formula (I)+4-(2′,4′-dichloro-4-ethylbiphenyl-3-yl)-2,2,6,6-tetramethyl-2H-pyran-3,5(4H,6H)-dione (which is compound A-45 disclosed on page 93 of WO 2008/071405 A1 (Syngenta Participations AG and Syngenta Limited), and which is also the compound of Example P10 disclosed on pages 41 and 45 of WO 2010/136431 A9 (Syngenta Limited), and which is also compound A-7 disclosed on page 7 of WO 2011/073615 A2 (Syngenta Limited), these parts of these publications being incorporated herein by reference), or compound of formula (I)+4-(2′,4′-dichloro-4-ethylbiphenyl-3-yl)-5-(methoxycarbonyloxy)-2,2,6,6-tetramethyl-2H-pyran-3(6H)-one (which is compound D-26 disclosed on page 231 of WO 2008/071405 A1 (Syngenta Participations AG and Syngenta Limited), and which is also compound A-9 disclosed on page 8 of WO 2011/073615 A2 (Syngenta Limited), these parts of these publications being incorporated herein by reference);
wherein the mixture partners for the compound of formula (I) may optionally be in the form of an ester (in particular an agrochemically acceptable ester) or a salt (in particular an agrochemically acceptable salt) thereof (e.g. where chemically possible).

In the above-mentioned compositions or mixtures comprising a compound of formula (I) (in particular, one of the specific compounds disclosed herein, e.g. any of compounds A1 to A95 or P1 to P7 and/or any of the compounds disclosed in Tables 1 to 47 herein, present either as a free compound and/or as an agrochemically acceptable salt thereof) and one or more further herbicides, the weight ratio of the compound of formula (I) to each further herbicide can vary over a large range and is, typically, from 300:1 to 1:500, especially from 150:1 to 1:200, more especially from 100:1 to 1:100, even more especially from 30:1 to 1:30. Typically, these weight ratios are measured as the free compound(s), i.e. excluding the weight of any associated salt counterion(s).

The compounds of formula I according to the invention can also be used in combination with a safener. Preferably, in these mixtures, the compound of the formula I is one of the specific compounds disclosed herein, in particular one of those compounds listed (disclosed) in Tables 1 to 47 and/or one of the exemplified compounds (e.g. one of compounds A1 to A95 or P1 to P7) below. The following mixtures with safeners, especially, come into consideration:

compound of formula I+cloquintocet-mexyl, compound of formula I+cloquintocet acid or an agrochemically acceptable salt thereof, compound of formula I+fenchlorazole-ethyl, compound of formula I+fenchlorazole acid or an agrochemically acceptable salt thereof, compound of formula I+mefenpyr-diethyl, compound of formula I+mefenpyr diacid, compound of formula I+isoxadifen-ethyl, compound of formula I+isoxadifen acid, compound of formula I+furilazole, compound of formula I+furilazole R isomer, compound of formula (I)+N-(2-methoxybenzoyl)-4-[(methylaminocarbonyl)amino]benzenesulfonamide, compound of formula I+benoxacor, compound of formula I+dichlormid, compound of formula I+AD-67, compound of formula I+oxabetrinil, compound of formula I+cyometrinil, compound of formula I+cyometrinil Z-isomer, compound of formula I+fenclorim, compound of formula I+cyprosulfamide, compound of formula I+naphthalic anhydride, compound of formula I+flurazole, compound of formula I+CL 304,415, compound of formula I+dicyclonon, compound of formula I+fluxofenim, compound of formula I+DKA-24, compound of formula I+R-29148 and compound of formula I+PPG-1292.

The above-mentioned safeners and herbicides are described, for example, in the Pesticide Manual, 14^{th} Edition, British Crop Protection Council, 2006; or The Pesticide Manual, 15^{th} edition, (2009) or 16th Edition (2012), ed. C. D. S. Tomlin, British Crop Production Council. R-29148 is described, for example by P. B. Goldsbrough et al., Plant Physiology, (2002), Vol. 130 pp. 1497-1505 and references therein. PPG-1292 is known from WO 2009/211761. N-(2-methoxybenzoyl)-4-[(methylaminocarbonyl)amino]benzenesulfonamide is known from EP365484.

Especially preferably, in a composition or mixture comprising a compound of formula (I) (in particular, one of the specific compounds disclosed herein, e.g. any of compounds A1 to A95 or P1 to P7 and/or any of the compounds disclosed in Tables 1 to 47 herein, present either as a free compound and/or as an agrochemically acceptable salt thereof) and a safener, the safener comprises (e.g. is) benoxacor, cloquintocet-mexyl, cloquintocet acid or an agrochemically acceptable salt thereof, cyprosulfamide, mefenpyr-diethyl, isoxadifen-ethyl and/or N-(2-methoxybenzoyl)-4-[(methylaminocarbonyl)amino]-benzenesulfonamide. In one particular embodiment, the safener comprises (e.g. is) cloquintocet-mexyl, cloquintocet acid or an agrochemically acceptable salt thereof, mefenpyr-diethyl and/or isoxadifen-ethyl; in particular for use on non-oat cereals such as wheat, barley, rye and/or triticale. Cloquintocet-mexyl is particularly valuable and is the most preferred safener, especially for use on non-oat cereals such as wheat, barley, rye and/or triticale.

In the above-mentioned compositions or mixtures comprising a compound of formula (I) (in particular, one of the specific compounds disclosed herein, e.g. any of compounds A1 to A95 or P1 to P7 and/or any of the compounds disclosed in Tables 1 to 47 herein, present either as a free compound and/or as an agrochemically acceptable salt thereof) with a safener, the weight ratio of the compound of formula (I) to the safener can vary over a large range and is, typically, from 200:1 to 1:200, especially from 50:1 to 1:50 such as from 50:1 to 1:20, more especially from 20:1 to 1:20, even more especially from 20:1 to 1:10. Preferably, the safener comprises (e.g. is) benoxacor, cloquintocet-mexyl, cloquintocet acid or an agrochemically acceptable salt thereof, cyprosulfamide, mefenpyr-diethyl, isoxadifen-ethyl and/or N-(2-methoxybenzoyl)-4-[(methylaminocarbonyl)amino]-benzenesulfonamide, and the weight ratio of the compound of formula (I) to the safener is from 50:1 to 1:20 or from 20:1 to 1:10, more preferably from 15:1 to 1:2. Typically, these weight ratios are measured as the free compound(s), i.e. excluding the weight of any associated salt counterion(s).

Application rates of herbicide (e.g. compound of formula (I)) and/or safener: The rate of application of safener relative to the compound of formula (I) is largely dependent upon the mode of application. In the case of field and/or soil and/or plant treatment (e.g. in a field or glasshouse): for example from 0.5 to 1000 g of safener per ha, or preferably from 1 to 250 g or from 2 to 200 g of safener per ha, are applied; and/or generally from 1 to 2000 g of compound of formula (I) per ha, or preferably from 5 to 500 g or from 10 to 400 g of compound of formula (I) per ha, are applied. ha=hectare. Typically, these application rates are measured as the free compound, i.e. excluding the weight of any associated salt counterion(s). In field treatment, the application of the compound of formula (I) is preferably post-emergence.

The compounds and/or herbicidal compositions according to the invention are suitable for all methods of application customary in agriculture, such as, for example, pre-emergence application, post-emergence application and seed dressing. Post-emergence application is preferred. Depending upon the intended use, the safeners can be used for pretreating the seed material of the crop plant (dressing the seed or seedlings) or introduced into the soil before or after sowing, followed by the application of the (unsafened) compound of the formula (I), optionally in combination with a co-herbicide. It can, however, also be applied alone or together with the herbicide before or after emergence of the plants. The treatment of the plants or the seed material with the safener can therefore take place in principle independently of the time of application of the herbicide. The treatment of the plant by simultaneous application of herbicide and safener (e.g. in the form of a tank mixture) is generally preferred. The rate of application of safener relative to herbicide is largely dependent upon the mode of application. In the case of field and/or soil and/or plant treatment (e.g. in a field or glasshouse), generally from 0.001 to 5.0 kg of safener/ha, preferably from 0.001 to 0.5 kg of safener/ha, are applied. In the case of seed dressing, generally from 0.001 to 10 g of safener/kg of seed, preferably from 0.05 to 2 g of safener/kg of seed, are applied. When the safener is applied in liquid form, with seed soaking, shortly before sowing, it is advantageous to use safener solutions which contain the active ingredient in a concentration of from 1 to 10 000 ppm, preferably from 100 to 1000 ppm.

In the invention, in the case of field and/or soil and/or plant treatment (e.g. post-emergence application), generally from 1 to 2000 g of herbicide (in particular compound of formula (I))/ha, but preferably from 5 to 1000 g of herbicide (in particular compound of formula (I))/ha, more preferably from 10 to 400 g of herbicide (in particular compound of formula (I))/ha, is applied. If a safener is used, in the case of field and/or soil and/or plant treatment (e.g. post-emergence application), generally from 0.5 to 1000 g of safener/ha, preferably from 2 to 500 g of safener/ha, more preferably from 5 to 200 g of safener/ha, is applied.

In one particular embodiment, the composition or mixture comprising the compound of formula (I) and one or more further herbicides (e.g. as mentioned hereinabove) can be applied together with one of the safeners mentioned herein, e.g. hereinabove.

In a further aspect, the present invention provides a herbicidal composition, e.g. for use in a method of controlling weeds (in particular monocotyledonous such as grassy monocotyledonous weeds) in crops of useful plants, comprising a compound of formula (I) as defined herein (in particular, one of the specific compounds disclosed herein, in particular one of compounds A1 to A95 or P1 to P7 disclosed herein, present either as a free compound and/or as an agrochemically acceptable salt thereof) (e.g. a herbicidally effective amount thereof), and an agrochemically acceptable carrier, diluent and/or solvent, and also comprising a plant growth regulator, and optionally one or more further herbicides (e.g. as described herein, e.g. glyphosate and/or dicamba and/or 2,4-D) and optionally a safener (e.g. as described herein).

Preferably, the plant growth regulator is: abscisic acid, acibenzolar-S-methyl, a brassinosteroid plant growth regulator, 24-epi brassinolide, 28-homobrassinolide, chlormequat, a cytokinin plant growth regulator, ethephon, ethylene, flurprimidol, gibberellic acid, a gibberellin plant growth regulator, GR24, indole-3-acetic acid (IAA), indole-3-butyric acid (IBA), jasmonic acid, methyl jasmonate, a karrikin plant growth regulator, maleic hydrazide, mefluidide, mepiquat, methylcyclopropene such as 1-methylcyclopropene, 1-naphthaleneacetic acid (NAA), paclobutrazol, prohexadione, prohexadione-calcium, salicylic acid, a strigolactone plant growth regulator (such as strigol or orobanchol or a derivative of one of these, or the synthetic strigolactone GR-24) (see e.g. K. Yoneyma et al, “Strigolactones as a new plant growth regulator”, http://www.niaes.affrc.go.jp/marco/marco2009/english/program/W3-04_Yoneyama_Koichi.pdf), trinexapac-ethyl and/or uniconzole, or an agrochemically acceptable salt e.g. acid addition salt or metal or ammonium salt e.g. alkali metal salt of any of these. More preferably, the plant growth regulator is: gibberellic acid, or a gibberellin plant growth regulator, or an agrochemically acceptable salt e.g. metal or ammonium salt e.g. alkali metal salt of any of these. Most preferably, the plant growth regulator is gibberellic acid or an agrochemically acceptable salt e.g. metal or ammonium salt e.g. alkali metal salt thereof. Gibberellic acid is preferred because WO 2014/071110 A1 discloses that gibberelic acid, when mixed with clethodim, increased clethodim's control and/or speed of control of Johnsongrass (Sorghum halepense) and volunteer corn; and increased the control of glyphosate-tolerant (Roundup-Ready™) volunteer corn at 21 days after the application of a mixture of clethodim+dicamba-glycolamine+glyphosate+gibberellic acid (compared to clethodim+dicamba-glycolamine+glyphosate+ammonium sulfate).

In the above-mentioned herbicidal compositions comprising a compound of formula (I), an agrochemically acceptable carrier, diluent and/or solvent, and a plant growth regulator (e.g. gibberellic acid or a salt thereof), and optionally one or more further herbicides and optionally a safener, the weight ratio of the compound of formula (I) to the plant growth regulator (e.g. gibberellic acid or an agrochemically acceptable salt e.g. metal salt e.g. alkali metal salt thereof) can vary over a large range and is, typically, from 500:1 to 1:500, especially from 200:1 to 1:200, more especially from 100:1 to 1:100, even more especially from 30:1 to 1:30. Typically, these weight ratios are measured as the free compound(s), i.e. excluding the weight of any associated salt counterion(s).

The following Examples illustrate the invention further but do not limit the invention.

### PREPARATION EXAMPLES

Those skilled in the art will appreciate that certain compounds described below are mostly beta-ketoenols, and as such may exist as a single tautomer or as a mixture of keto-enol and diketone tautomers, as described and/or shown in the description hereinabove, and/or as described for example by J. March, Advanced Organic Chemistry, third edition, John Wiley and Sons. The compounds shown below, and/or in Table T1 and/or Table P1 and/or in Tables 1 to 47 hereinafter, are drawn as an arbitrary single tautomer (usually as the diketone, occasionally as the keto-enol), but it should be inferred that this description covers both the diketone (cyclic dione) form and any possible enols/keto-enols which could arise through tautomerism. Where more than one tautomer is observed in proton (¹H) NMR, the data shown are for the mixture of tautomers. Furthermore, for compounds shown below which can exist as enantiomers, unless specified as single enantiomers, these structures are often formed as a mixture of enantiomers, for example a substantially racemic mixture of enantiomers. Additionally, some of the compounds can exist as diastereoisomers, and it should be inferred that these can be present as a mixture of diastereoisomers or as any possible single diastereoisomer. Within the detailed experimental section the diketone tautomer is usually chosen for naming purposes, even if the predominant tautomer is the enol form.

### Abbreviations

EtOAc—ethyl acetate
THF—tetrahydrofuran
Room/ambient temperature=usually this is about 15-30° C., e.g. about 15-25° C.
NMR=nuclear magnetic resonance; in which: s=singlet, d=doublet, t=triplet, q=quartet,
dd=doublet of doublets, brs=broad singlet, m=multiplet.
d4 MeOD=tetradeutero-methanol (CD₃OD)

### Example 1

### Synthesis of 3-(2,6-dimethyl-4-prop-1-ynyl-phenyl)-1-methyl-5-(2-pyridylmethyl)pyrrolidine-2,4-dione

### Step 1. Synthesis of methyl 2-(tert-butoxycarbonylamino)-3-(2-pyridyl)prop-2-enoate

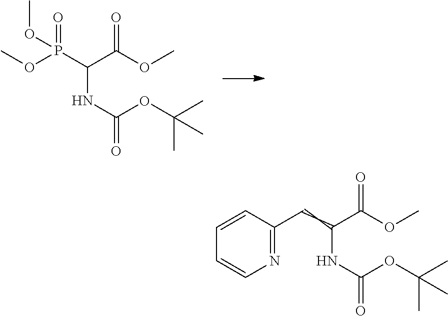 [SMILES: COC(=O)C(NC(=O)OC(C)(C)C)P(=O)(OC)OC ; COC(=O)C(=Cc1ccccn1)NC(=O)OC(C)(C)C]

1,1,3,3-Tetramethylguanidine (18 mmol, 2.3 ml) was added dropwise over one minute to a stirred solution of methyl 2-(tert-butoxycarbonylamino)-2-dimethoxyphosphoryl-acetate (16.9 mmol, 5.03 g) and 2-pyridinecarboxaldehyde (17.7 mmol, 1.91 g) in tetrahydrofuran (50 ml). The mixture was stirred for two hours; then the tetrahydrofuran was removed under reduced pressure. The residue was partitioned between water (100 ml) and ethyl acetate (100 ml). The phases were separated and the organic layer was washed sequentially with water (100 ml), saturated aqueous NH₄CI (30 ml) and brine (50 ml). The organic layer was dried over MgSO₄ and concentrated under reduced pressure to give the desired compound (4.70 g, 95%) as a viscous, yellow oil. ¹H NMR (400 MHz, CDCl₃) (diagnostic peaks) δ (delta) ppm 1.49 (s, 9H) 3.88 (s, 3H) 6.19 (s, 1H) 7.15 (ddd, J=7.5, 5.0, 1.0 Hz, 1H) 7.20 (d, J=7.8 Hz, 1H) 7.66 (td, J=7.8, 1.8 Hz, 1H) 8.59 (d, J=3.9 Hz, 1H) 10.49-11.19 (m, 1H).

### Step 2. Synthesis of methyl 2-[tert-butoxycarbonyl(methyl)amino]-3-(2-pyridyl)prop-2-enoate

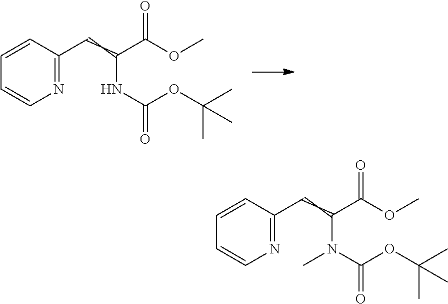 [SMILES: COC(=O)C(=Cc1ccccn1)NC(=O)OC(C)(C)C ; COC(=O)C(=Cc1ccccn1)N(C)C(=O)OC(C)(C)C]

Methyl 2-(tert-butoxycarbonylamino)-3-(2-pyridyl)prop-2-enoate (9.22 mmol, 2.70 g) was combined with crushed potassium carbonate (45.4 mmol, 6.27 g) and iodomethane (47 mmol, 2.9 ml) in N,N-dimethylformamide (15 ml). The mixture was stirred vigorously at room temperature for 20 hours and then partitioned between ethyl acetate (100 ml) and water (100 ml). The phases were separated and the aqueous layer was extracted with ethyl acetate (2×25 ml). The combined organic layers were washed with water (3×100 ml) then brine (50 ml), then dried over MgSO₄ and concentrated under reduced pressure. The residue was purified by column chromatography on SiO₂ along a hexane: ethyl acetate gradient to yield the desired compound (1.12 g, 42%) as a yellow oil. ¹H NMR (400 MHz, CDCl₃) (diagnostic peaks for a 0.17:0.83 mixture of carbamate rotamers) δ (delta) ppm 1.35 (s, 7.5H) 1.51 (s, 1.5H) 2.90-3.03 (m, 3H) 3.79-3.95 (m, 3H) 7.20-7.26 (m, 1H) 7.43 (s, 0.83H) 7.53 (s, 0.17H) 7.60 (d, J=8.1 Hz, 1H) 7.67-7.79 (m, 1H) 8.60-8.74 (m, 1H).

### Step 3. Synthesis of methyl 2-[tert-butoxycarbonyl(methyl)amino]-3-(2-pyridyl)prop-2-enoate

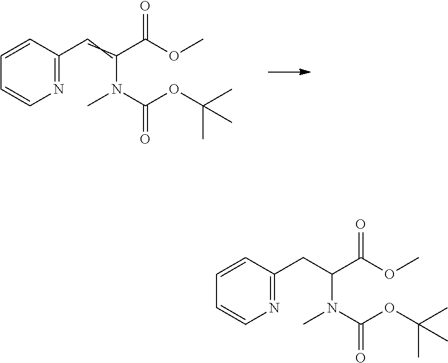 [SMILES: COC(=O)C(=Cc1ccccn1)N(C)C(=O)OC(C)(C)C ; COC(=O)C(Cc1ccccn1)N(C)C(=O)OC(C)(C)C]

An autoclave was charged with methyl 2-[tert-butoxycarbonyl(methyl)amino]-3-(2-pyridyl)prop-2-enoate (3.25 mmol, 950 mg), palladium on carbon (0.09 mmol, 5 mass %, 0.2 g) and methanol (20 ml). The vessel was pressurised to 3.5 Bar with hydrogen and stirred at room temperature for three hours. The mixture was filtered through Celite™ to remove the catalyst and the filtrate was concentrated under reduced pressure. The residue was purified by column chromatography on SiO₂ along a hexane: ethyl acetate gradient to yield the desired compound (449 mg, 47%) as a yellow oil. ¹H NMR (400 MHz, CDCl₃) (diagnostic peaks for a 2:3 mixture of carbamate rotamers) δ (delta) ppm 1.37 (s, 9H) 2.77 (s, 3H) 3.14-3.31 (m, 1H) 3.42-3.56 (m, 1H) 3.75 (s, 3H) 4.83 (dd, J=10.1, 4.8 Hz, 0.6H) 5.04 (dd, J=10.0, 5.3 Hz, 0.4H) 7.06-7.24 (m, 2H) 7.53-7.68 (m, 1H) 8.54 (d, J=4.5 Hz, 1H).

### Step 4. Synthesis of [2-methoxy-2-oxo-1-(pyridin-1-ium-2-ylmethyl)ethyl]-methyl-ammonium bis(2,2,2-trifluoroacetate)

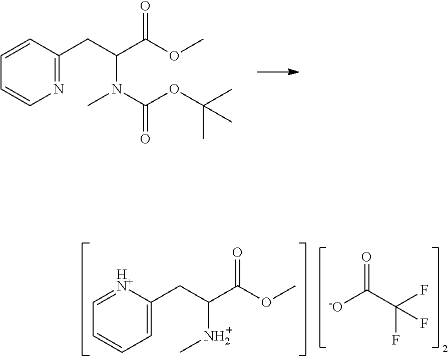

A solution of methyl 2-[tert-butoxycarbonyl(methyl)amino]-3-(2-pyridyl)propanoate (3.09 mmol, 910 mg) in dichloromethane (8 ml) was cooled to 0° C., before dropwise addition of 2,2,2-trifluoroacetic acid (2 ml). The mixture was then warmed to room temperature and stood for 20 hours, then was concentrated under reduced pressure. The residue was azeotroped three times with water, then twice with acetonitrile to give the crude desired compound (1.41 g, assumed 100%) as a yellow gum, which was used without any further purification.

### Step 5. Synthesis of methyl 2-[[2-(2,6-dimethyl-4-prop-1-ynyl-phenyl)acetyl]-methyl-amino]-3-(2-pyridyl)propanoate

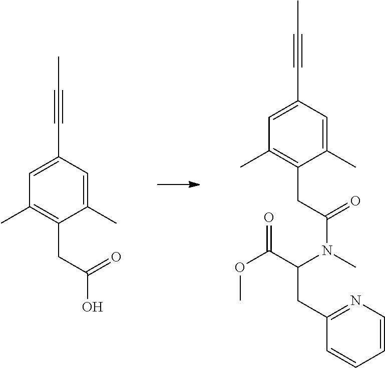 [SMILES: CC#Cc1cc(C)c(CC(=O)O)c(C)c1 ; CC#Cc1cc(C)c(CC(=O)N(C)C(Cc2ccccn2)C(=O)OC)c(C)c1]

Triethylamine (2.4 mmol, 0.34 ml) was added dropwise to a stirred solution containing crude [2-methoxy-2-oxo-1-(pyridin-1-ium-2-ylmethyl)ethyl]-methyl-ammonium bis(2,2,2-trifluoroacetate) (approximately 0.55 mmol, 250 mg), N,N-dimethylaminopyridine (0.39 mmol, 48 mg) and 2-(2,6-dimethyl-4-prop-1-ynyl-phenyl)acetic acid (0.41 mmol, 83 mg) in N,N-dimethylformamide (2 ml) at 0° C. A solution of 1-propanephosphonic anhydride (0.71 ml of a 50% solution in ethyl acetate, 1.2 mmol) was then added dropwise to the mixture. After five minutes, the mixture was warmed to room temperature and stirred for one hour, then added to saturated aqueous NaHCO₃ (15 ml). The mixture was stirred rapidly for 10 min then extracted with ethyl acetate (2×15 ml). The combined organic layers were washed with water (3×20 ml) and brine (10 ml), then dried over MgSO₄ and concentrated under reduced pressure. The residue was purified by chromatography on SiO₂ along a hexane: ethyl acetate gradient to yield the desired compound (105 mg, 68%) as a pale brown gum. ¹H NMR (400 MHz, CDCl₃) (diagnostic peaks for a 2:1 mixture of amide rotamers) δ (delta) ppm 2.01 (s, 1H) 2.01 (s, 3H) 2.03 (s, 3H) 2.04 (s, 2H) 2.84 (s, 1H) 3.07 (s, 2H) 3.24 (dd, J=15.0, 10.5 Hz, 0.33H) 3.32 (dd, J=14.6, 11.0 Hz, 0.67H) 3.44 (d, J=16.8 Hz, 1H) 3.48-3.66 (m, 2H) 3.73 (s, 2H) 3.76 (d, J=17.7 Hz, 0.33H) 3.80 (s, 1H) 5.34 (dd, J=10.9, 5.0 Hz, 0.67H) 5.52 (dd, J=10.4, 4.5 Hz, 0.33H) 7.01 (s, 2H) 7.11-7.23 (m, 2H) 7.54-7.66 (m, 1H) 8.52 (d, J=4.2 Hz, 0.67H) 8.57 (d, J=4.8 Hz, 0.33H).

### Step 6.—Synthesis of 3-(2,6-dimethyl-4-prop-1-ynyl-phenyl)-1-methyl-5-(2-pyridylmethyl)pyrrolidine-2,4-dione (=compound A50 in Table T1 hereinafter)

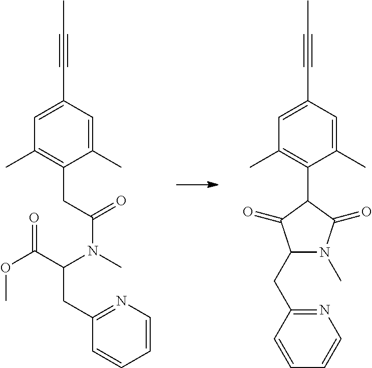 [SMILES: CC#Cc1cc(C)c(CC(=O)N(C)C(Cc2ccccn2)C(=O)OC)c(C)c1 ; CC#Cc1cc(C)c(C2C(=O)C(Cc3ccccn3)N(C)C2=O)c(C)c1]

A mixture of potassium tert-butoxide (0.30 ml of a 1.0M solution in THF, 0.30 mmol) and toluene (2.5 ml) was cooled to 0° C., before a solution of methyl 2-[[2-(2,6-dimethyl-4-prop-1-ynyl-phenyl)acetyl]-methyl-amino]-3-(2-pyridyl)propanoate (0.272 mmol, 103 mg) in toluene (2.5 ml) was added dropwise. The mixture was stirred whilst being allowed to warm to room temperature over one hour, then it was partitioned with saturated aqueous NaHCO₃ (5 ml) and the phases were separated. The pH of the aqueous layer was adjusted to 6 by gradual addition of 2M aqueous HCl, then the mixture was extracted with ethyl acetate (4×5 ml). The combined organic layers were dried over MgSO₄ and then concentrated under reduced pressure. The residue was purified by column chromatography on SiO₂ along a hexane: ethyl acetate gradient to yield the desired compound (36 mg, 38%) as a yellow glass. ¹H NMR (400 MHz, CDCl₃) (diagnostic peaks) δ (delta) ppm 2.03 (s, 3H) 2.19 (s, 3H) 2.23 (s, 3H) 3.04 (s, 3H) 3.17 (dd, J=16.1, 11.0 Hz, 1H) 3.51 (dd, J=16.1, 1.7 Hz, 1H) 4.20 (dd, J=11.0, 1.7 Hz, 1H) 7.08 (s, 1H) 7.10 (s, 1H) 7.29-7.36 (m, 1H) 7.44 (d, J=7.8 Hz, 1H) 7.75-7.93 (m, 1H) 8.47 (d, J=4.5 Hz, 1H).

### Example 2

### Synthesis of 3-(2,6-dimethyl-4-prop-1-ynyl-phenyl)-1-(2-pyridylmethyl)pyrrolidine-2,4-dione

### Step 1. Synthesis of methyl 2-[(pyridin-2-ylmethyl)amino]acetate dihydrochloride

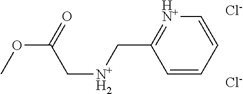

Pyridine-2-carbaldehyde (1.39 g, 13.0 mmol) was combined with methyl 2-aminoacetate hydrochloride (1.71 g, 13.7 mmol) in 1,2-dichloroethane (45 mL). Triethylamine (1.9 mL, 14 mmol) was added dropwise and the mixture was stirred at room temperature for 10 min. sodium triacetoxyborohydride (3.85 g, 18.2 mmol, 100 mass %) was then added portionwise over 1 min. The mixture was stirred for 1 h, then filtered through Celite, rinsing with DCM. The filtrate was concentrated under reduced pressure and the residue was triturated with ethyl acetate/hexanes to remove solids, then the filtrate was concentrated under reduced pressure and purified by flash chromatography over silica using a 0.1% to 10% methanol/DCM gradient to give an intermediate acetate salt. This was dissolved in a 2 M solution of HCl in methanol (20 mL, 40 mmol), then the solution was concentrated under reduced pressure. The resulting solid was triturated with acetonitrile and ethyl acetate to give the desired product (1.48 g, 45%) as a cream-coloured solid. ¹H NMR (400 MHz, DMSO-d₆) δ=9.98 (br. s., 1H), 8.71-8.63 (m, 1H), 7.97 (dt, 1H), 7.63 (d, 1H), 7.51 (dd, 1H), 4.91 (br. s., 2H), 4.40 (s, 2H), 4.14-4.04 (m, 2H), 3.75 (s, 3H).

### Step 2: Synthesis of methyl 2-[[2-(4-bromo-2,6-dimethyl-phenyl)acetyl]-(2-pyridylmethyl)amino]acetate

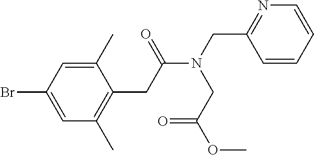 [SMILES: COC(=O)CN(Cc1ccccn1)C(=O)Cc1c(C)cc(Br)cc1C]

Methyl 2-(2-pyridylmethylamino)acetate dihydrochloride (1.00 g, 3.95 mmol) was combined with 4-(dimethylamino)pyridine (508 mg, 4.16 mmol) and 2-(4-bromo-2,6-dimethylphenyl)acetic acid (888 mg, 3.65 mmol) in N,N-dimethylformamide (16 mL). The mixture was stirred at room temperature as triethylamine (3.5 mL, 25 mmol) was added dropwise, then cooled to 0° C. before dropwise addition of a solution of 1-propanephosphonic anhydride (50% in ethyl acetate, 7.5 mL, 13 mmol). After 5 min, the ice bath was removed and the mixture was warmed to room temperature and stirred for 1 h, then the mixture was added dropwise to a rapidly-stirred solution of saturated aqueous NaHCO₃ (100 mL) and the resulting mixture was stirred for 20 min. The mixture was then extracted with ethyl acetate (25 mL) and the extracts were washed sequentially with saturated aqueous ammonium chloride (50 mL), water (50 mL) and brine (50 mL), then dried (MgSO₄) and concentrated under reduced pressure. The residue was purified by flash chromatography over silica using a 50% to 100% ethyl acetate/isohexane gradient to give the desired product (1.22 g, 82% Yield) as a pale brown gum. Data for major rotamer: ¹H NMR (400 MHz, CDCl₃) δ=8.62 (d, 1H), 7.74 (dt, 1H), 7.33 (d, 1H), 7.29-7.24 (m, 1H), 7.15 (s, 2H), 4.83 (s, 2H), 4.19 (s, 2H), 3.78 (s, 2H), 3.71 (s, 3H), 2.18 (s, 6H). Data for minor rotamer: ¹H NMR (400 MHz, CDCl₃) δ=8.50 (d, 1H), 7.64 (dt, 1H), 7.35 (d, 1H), 7.20 (d, 1H), 7.17 (s, 2H), 4.73 (s, 2H), 4.35 (s, 2H), 3.74 (s, 3H), 3.59 (s, 2H), 2.20 (s, 6H).

### Step 3: Synthesis of methyl 2-[[2-(2,6-dimethyl-4-prop-1-ynyl-phenyl)acetyl]-(2-pyridylmethyl)amino]acetate

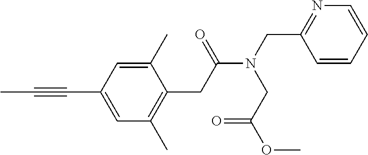 [SMILES: CC#Cc1cc(C)c(CC(=O)N(CC(=O)OC)Cc2ccccn2)c(C)c1]

Methyl 2-[[2-(4-bromo-2,6-dimethyl-phenyl)acetyl]-(2-pyridylmethyl)amino]acetate (300 mg, 0.740 mmol) was combined with bis(triphenylphosphine)palladium(II) chloride (26 mg, 0.037 mmol), 1,4-bis(diphenylphospino)butane (32 mg, 0.075 mmol). A solution of tetra-n-butylammonium fluoride solution in dimethyldulfoxide (1.1 mmol in 5 mL) was added and the mixture was degassed by purging with nitrogen, then heated at 90° C. for 1.5 h. Further 1,4-bis(diphenylphospino)butane (32 mg, 0.075 mmol) and bis(triphenylphosphine)palladium(II) chloride (26 mg, 0.037) were then added and the mixture was heated at 100° C. for a further hour. The mixture was cooled to room temperature then partitioned between water (30 mL) and ethyl acetate (25 mL). The phases were separated and the aqueous layer was extracted with ethyl acetate (2×20 mL). The combined organic layers were washed with water (3×20 mL), then brine (20 mL), then dried (MgSO₄) and concentrated under reduced pressure. The residue was purified by flash chromatography over silica using a 25% to 100% ethyl acetate/isohexane gradient to give the desired product (125 mg, 46%) as a light brown gum that partially crystallised on standing. Data for major rotamer: ¹H NMR (400 MHz, CDCl₃) δ=8.61 (d, 1H), 7.73 (dt, 1H), 7.32 (d, 1H), 7.29-7.23 (m, 1H), 7.05 (s, 2H), 4.84 (s, 2H), 4.20 (s, 2H), 3.80 (s, 2H), 3.71 (s, 3H), 2.17 (s, 6H), 2.02 (s, 3H). Data for minor rotamer: ¹H NMR (400 MHz, CDCl₃) δ=8.50 (d, 1H), 7.66-7.62 (m, 1H), 7.36 (d, 1H), 7.19 (dd, 1H), 7.07 (s, 2H), 4.73 (s, 2H), 4.33 (s, 2H), 3.73 (s, 3H), 3.62 (s, 2H), 2.19 (s, 6H), 2.02 (s, 3H).

### Step 4: Synthesis of 3-(2,6-dimethyl-4-prop-1-ynyl-phenyl)-1-(2-pyridylmethyl)pyrrolidine-2,4-dione

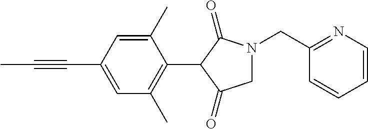 [SMILES: CC#Cc1cc(C)c(C2C(=O)CN(Cc3ccccn3)C2=O)c(C)c1]

Methyl 2-[[2-(2,6-dimethyl-4-prop-1-ynyl-phenyl)acetyl]-(2-pyridylmethyl)amino]acetate (118 mg, 0.381 mmol) was dissolved in N,N-dimethylformamide under nitrogen then a solution of potassium t-butoxide in THF (1 M, 0.76 mL, 0.76 mmol) was added dropwise at room temperature. The mixture was stirred for 2 h, then it was added dropwise to a solution of saturated aqueous NH₄CI (30 mL). 2 M aqueous HCl was added to adjust the pH to 4 and the mixture was extracted with ethyl acetate (3×20 mL), then the combined organic layers were washed with water (2×20 mL) and brine (20 mL), then dried (MgSO₄) and concentrated under reduced pressure. The residue was triturated with ether/ethyl acetate, then with acetonitrile. Filtration and air-drying under vacuum gave the desired product (69 mg, 64%) as a tan-coloured solid. ¹H NMR (400 MHz, d4-methanol) δ=8.51 (d, 1H), 7.83 (dt, 1H), 7.38-7.29 (m, 2H), 7.07 (s, 2H), 4.74 (s, 2H), 4.59 (br. s., 1H), 4.06 (s, 2H), 2.16 (s, 6H), 2.00 (s, 3H).

### Example 3

### Synthesis of [2-(2,6-dimethyl-4-prop-1-ynyl-phenyl)-1-oxo-6,7,8,9-tetrahydro-5H-pyrazolo[1,2-a]diazepin-3-yl]2,2-dimethylpropanoate

### Step 1: Synthesis of dimethyl 2-(2,6-dimethyl-4-prop-1-ynyl-phenyl)propanedioate

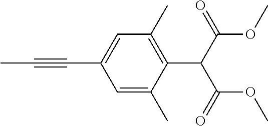 [SMILES: CC#Cc1cc(C)c(C(C(=O)OC)C(=O)OC)c(C)c1]

To a solution of dimethyl 2-(4-bromo-2,6-dimethyl-phenyl)propanedioate (1.45 g, 4.60 mmol) and tri-n-butyl(prop-1-ynyl)stannane (2.12 g, 6.44 mmol) in toluene (72.5 mL) was added [1,1′-Bis(diphenylphosphino)ferrocene]dichloropalladium(II) (150 mg, 0.184 mmol). The mixture was heated under reflux open to air for 4.5 h then cooled to room temperature. The mixture was partitioned between water and ethyl acetate. The phases were separated and the aqueous layer was extracted twice with ethyl acetate. The combined organic layers were washed with brine, then dried over MgSO₄ and concentrated under reduced pressure. The residue was purified by flash chromatography over silica using 50% ethyl acetate/isohexane to give the desired product (463 mg, 37% Yield) as a yellow solid. ¹H NMR (400 MHz, CDCl₃) δ (ppm) 7.10 (s, 2H), 5.02 (s, 1H), 3.76 (s, 6H), 2.30 (s, 6H), 2.03 (s, 3H).

### Step 2: Synthesis of 2-(2,6-dimethyl-4-prop-1-ynyl-phenyl)-6,7,8,9-tetrahydro-5H-pyrazolo[1,2-a]diazepine-1,3-dione

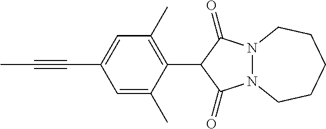 [SMILES: CC#Cc1cc(C)c(C2C(=O)N3CCCCCN3C2=O)c(C)c1]

A degassed suspension of diazepane dihydrochloride (1.74 g, 10.1 mmol) and triethylamine (8.38 mL, 60.2 mmol) in xylenes (33.7 mL) was stirred under nitrogen at 60° C. for 2.5 h. 5 A molecular sieves (1 g/g, 100 mass %, 0.660 g) and dimethyl 2-(2,6-dimethyl-4-prop-1-ynyl-phenyl)propanedioate (660 mg, 2.41 mmol) were then added and the reaction mixture was heated to 150° C. for 6 hours. The mixture was cooled to room temperature then poured into water (40 mL). 2 M aqueous sodium hydroxide (10 mL, 20 mmol) was then added and the phases were separated. The aqueous layer was extracted twice with ethyl acetate then the combined organic layers were extracted twice with 2 M aqueous NaOH. The organic layers were discarded and the combined aqueous layers were acidified to pH 2-3 by gradual addition of 2 M aqueous HCl. The aqueous layers were extracted twice with DCM, and the combined DCM layers were dried over MgSO₄ and concentrated under reduced pressure. The residue was purified by flash chromatography over silica using a 0% to 10% methanol/DCM gradient to give the desired product (433 mg, 58%) as a pale yellow solid. Data for major keto tautomer: ¹H NMR (DMSO-d₆, 500 MHz) δ (ppm) 7.15 (s, 1H), 7.06 (s, 1H), 5.07 (s, 1H), 3.71-3.79 (m, 4H), 2.30 (s, 3H), 2.10 (s, 3H), 1.95 (s, 3H), 1.54-1.87 (m, 6H). Data for minor enol tautomer: ¹H NMR (DMSO-d₆, 500 MHz) δ (ppm) 7.07 (s, 2H), 4.01 (dd, J=14.6, 7.8 Hz, 4H), 2.04 (s, 9H), 1.55-1.88 (m, 6H).

### Step 3: Synthesis of [2-(2,6-dimethyl-4-prop-1-ynyl-phenyl)-1-oxo-6,7,8,9-tetrahydro-5H-pyrazolo[1,2-a]diazepin-3-yl]2,2-dimethylpropanoate

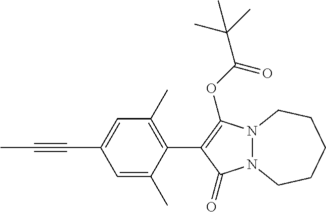 [SMILES: CC#Cc1cc(C)c(-c2c(OC(=O)C(C)(C)C)n3n(c2=O)CCCCC3)c(C)c1]

To a mixture of 2-(2,6-dimethyl-4-prop-1-ynyl-phenyl)-6,7,8,9-tetrahydro-5H-pyrazolo[1,2-a]diazepine-1,3-dione (206 mg, 0.664 mmol) and pyridine (0.064 mL, 0.796 mmol) in DCM (20 mL) was added pivaloyl chloride (0.0981 mL, 0.796 mmol). The mixture was stirred at room temperature for 2 h, then water was added and the mixture was extracted with DCM. The combined organic layers were dried over MgSO₄ and concentrated under reduced pressure. The residue was purified by flash chromatography over silica using a 0% to 10% methanol/DCM gradient to give the desired product (247 mg, 94%) as a yellow gum.

¹H NMR (500 MHz, CDCl₃) δ (ppm) 7.06 (s, 2H), 4.05-4.15 (m, 2H), 3.68-3.79 (m, 2H), 2.17 (s, 6H), 2.02 (s, 3H), 1.70-1.87 (m, 6H), 1.05 (s, 9H).

The additional compounds in Table T1 and Table P1 below illustrate the present invention, and are particular embodiments of the compounds of formula (I) according to the present invention. In most or many cases, these compounds are thought to be capable of being prepared by methods shown in, or methods similar or analogous to those shown in, the process section and/or the Examples hereinabove, using appropriate starting materials.

TABLE T1

| Com- | | |
|---|---|---|
| pound | | |
| number | Chemical structure | Physicochemical data, usually ¹H NMR |
| A1 | 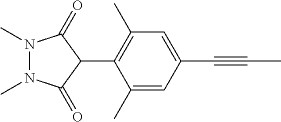 [SMILES: CC#Cc1cc(C)c(C2C(=O)N(C)N(C)C2=O)c(C)c1] | ¹H NMR (400 MHz, CDCl3): δ 7.13 (s, 2H), 3.18 (s, 6H), 2.23 (s, 6H), 2.04 (s, 3H). |
| A2 | 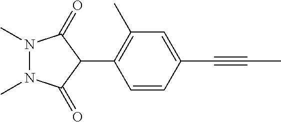 [SMILES: CC#Cc1ccc(C2C(=O)N(C)N(C)C2=O)c(C)c1] | ¹H NMR (500 MHz, CDCl3): δ 7.24 (s, 1 H) 7.19 (d, 1 H) 6.94 (d, 1 H) 4.41 (s, 1 H) 3.31 (s, 6 H) 2.31 (s, 3 H) 2.01 (s, 3 H)δ |
| A3 | 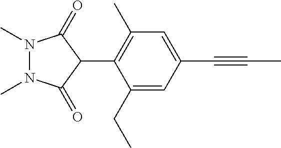 [SMILES: CC#Cc1cc(C)c(C2C(=O)N(C)N(C)C2=O)c(CC)c1] | |
| A4 | 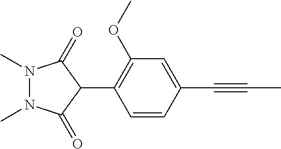 [SMILES: CC#Cc1ccc(C2C(=O)N(C)N(C)C2=O)c(OC)c1] | 1H NMR (500 MHz, Solvent) δ ppm 7.10-7.14 (m, 1 H) 6.97-7.02 (m, 1 H) 6.88 (d, 1 H) 4.13 (s, 1 H) 3.75 (s, 3 H) 3.25 (s, 6 H) 2.03 (s, 3 H) |
| A5 | 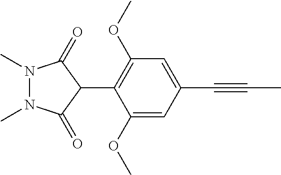 [SMILES: CC#Cc1cc(OC)c(C2C(=O)N(C)N(C)C2=O)c(OC)c1] | 1H NMR (500 MHz, CDCl3) δ 6.64 (s, 1 H) 6.54 (s, 1 H) 4.83 (s, 1 H) 3.82 (s, 3 H) 3.71 (s, 3 H) 3.22 (s, 6 H) 2.01 (s, 3 H). |
| A6 | 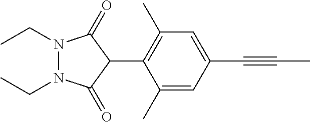 [SMILES: CC#Cc1cc(C)c(C2C(=O)N(CC)N(CC)C2=O)c(C)c1] | ¹H NMR (400 MHz, MeO)δ 7.09 (s, 2H), 3.76 (q, 4H), 2.14 (s, 6H), 2.00 (s, 3H), 1.09 (t, 6H) |
| A7 | 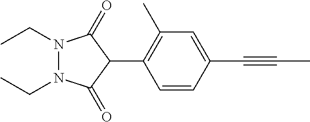 [SMILES: CC#Cc1ccc(C2C(=O)N(CC)N(CC)C2=O)c(C)c1] | |
| A8 | 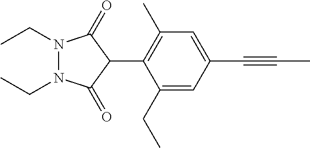 [SMILES: CC#Cc1cc(C)c(C2C(=O)N(CC)N(CC)C2=O)c(CC)c1] | |
| A9 | 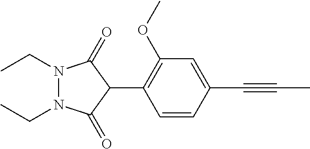 [SMILES: CC#Cc1ccc(C2C(=O)N(CC)N(CC)C2=O)c(OC)c1] | |
| A10 | 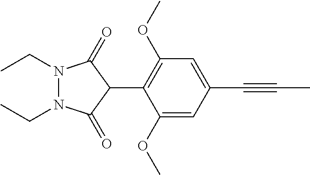 [SMILES: CC#Cc1cc(OC)c(C2C(=O)N(CC)N(CC)C2=O)c(OC)c1] | |
| A11 | 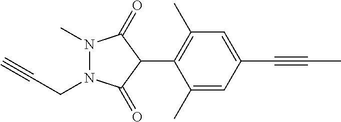 [SMILES: C#CCN1C(=O)C(c2c(C)cc(C#CC)cc2C)C(=O)N1C] | |
| A12 | 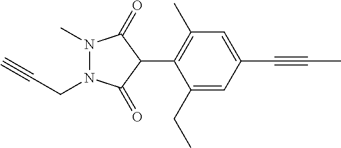 [SMILES: C#CCN1C(=O)C(c2c(C)cc(C#CC)cc2CC)C(=O)N1C] | |
| A13 | 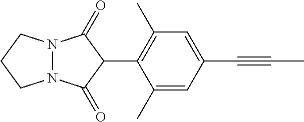 [SMILES: CC#Cc1cc(C)c(C2C(=O)N3CCCN3C2=O)c(C)c1] | |
| A14 | 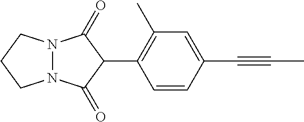 [SMILES: CC#Cc1ccc(C2C(=O)N3CCCN3C2=O)c(C)c1] | |
| A15 | 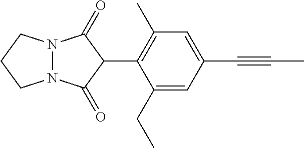 [SMILES: CC#Cc1cc(C)c(C2C(=O)N3CCCN3C2=O)c(CC)c1] | |
| A16 | 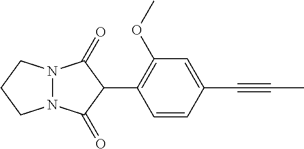 [SMILES: CC#Cc1ccc(C2C(=O)N3CCCN3C2=O)c(OC)c1] | |
| A17 | 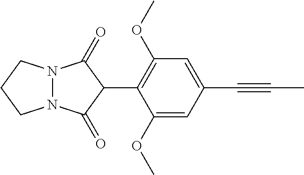 [SMILES: CC#Cc1cc(OC)c(C2C(=O)N3CCCN3C2=O)c(OC)c1] | |
| A18 | 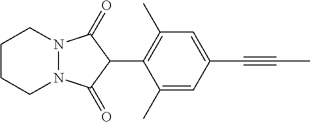 [SMILES: CC#Cc1cc(C)c(C2C(=O)N3CCCCN3C2=O)c(C)c1] | 1H NMR (400 MHz, d4-MeOD) δ 7.08 (s, 2H), 3.62-3.65 (br. m, 4H), 2.15 (s, 6H), 2.00 (s, 3H), 1.93-1.89 (br. m, 4H). |
| A19 | 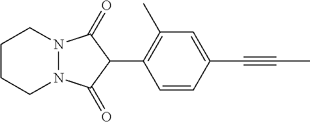 [SMILES: CC#Cc1ccc(C2C(=O)N3CCCCN3C2=O)c(C)c1] | |
| A20 | 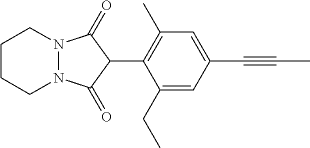 [SMILES: CC#Cc1cc(C)c(C2C(=O)N3CCCCN3C2=O)c(CC)c1] | |
| A21 | 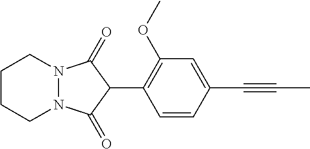 [SMILES: CC#Cc1ccc(C2C(=O)N3CCCCN3C2=O)c(OC)c1] | |
| A22 | 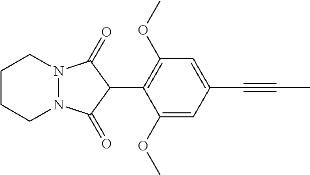 [SMILES: CC#Cc1cc(OC)c(C2C(=O)N3CCCCN3C2=O)c(OC)c1] | |
| A23 | 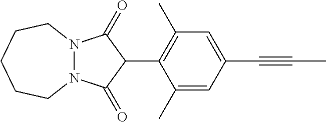 [SMILES: CC#Cc1cc(C)c(C2C(=O)N3CCCCCN3C2=O)c(C)c1] | 1H NMR (DMSO-d6, 500 MHz) peaks for major keto tautomer δ 7.15 (s, 1H), 7.06 (s, 1H), 5.07 (s, 1H), 3.71-3.79 (m, 4H), 2.30 (s, 3H), 2.10 (s, 3H), 1.95 (s, 3H), 1.54-1.87 (m, 6H); peaks for minor enol tautomer δ 7.07 (s, 2H), 4.01 (dd, 4H), 2.04 (s, 9H), 1.55-1.88 (m, 6H) |
| A24 | 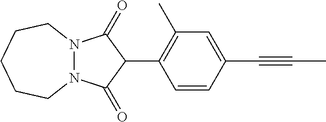 [SMILES: CC#Cc1ccc(C2C(=O)N3CCCCCN3C2=O)c(C)c1] | |
| A25 | 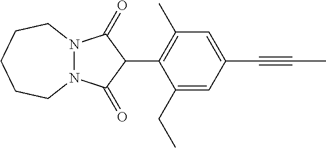 [SMILES: CC#Cc1cc(C)c(C2C(=O)N3CCCCCN3C2=O)c(CC)c1] | |
| A26 | 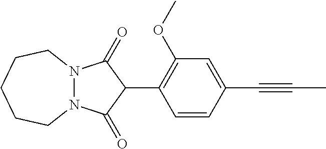 [SMILES: CC#Cc1ccc(C2C(=O)N3CCCCCN3C2=O)c(OC)c1] | |
| A27 | 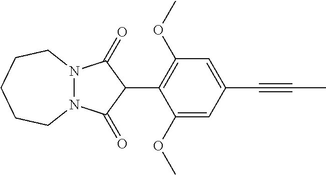 [SMILES: CC#Cc1cc(OC)c(C2C(=O)N3CCCCCN3C2=O)c(OC)c1] | |
| A28 | 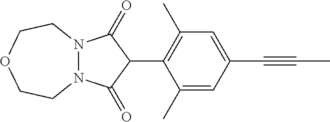 [SMILES: CC#Cc1cc(C)c(C2C(=O)N3CCOCCN3C2=O)c(C)c1] | 1H NMR (DMSO-d6, 500 MHz): major tautomer (keto) δ 7.08 (s, 2H), 5.14 (s, 1H), 3.69-3.90 (m, 8H), 2.12 (s, 3H), 2.04 (s, 3H), 1.92 (s, 3H); minor taoutomer (enol) δ 7.12-7.18 (m, 2H), 3.93- 4.11 (m, 8H), 2.31 (s, 6H), 1.98 (s, 3H) |
| A29 | 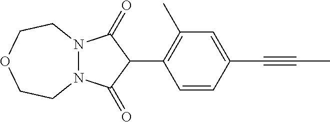 [SMILES: CC#Cc1ccc(C2C(=O)N3CCOCCN3C2=O)c(C)c1] | |
| A30 | 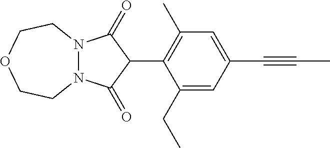 [SMILES: CC#Cc1cc(C)c(C2C(=O)N3CCOCCN3C2=O)c(CC)c1] | |
| A31 | 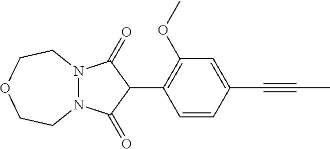 [SMILES: CC#Cc1ccc(C2C(=O)N3CCOCCN3C2=O)c(OC)c1] | |
| A32 | 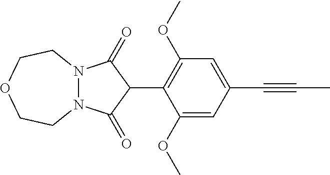 [SMILES: CC#Cc1cc(OC)c(C2C(=O)N3CCOCCN3C2=O)c(OC)c1] | |
| A33 | 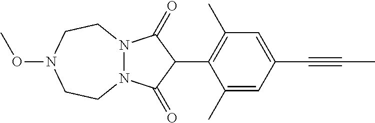 [SMILES: CC#Cc1cc(C)c(C2C(=O)N3CCN(OC)CCN3C2=O)c(C)c1] | |
| A34 | 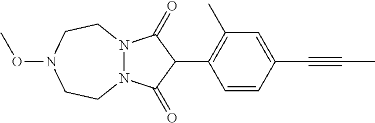 [SMILES: CC#Cc1ccc(C2C(=O)N3CCN(OC)CCN3C2=O)c(C)c1] | |
| A35 | 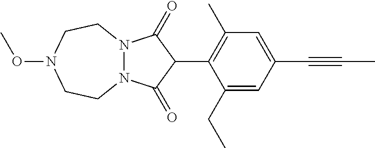 [SMILES: CC#Cc1cc(C)c(C2C(=O)N3CCN(OC)CCN3C2=O)c(CC)c1] | |
| A36 | 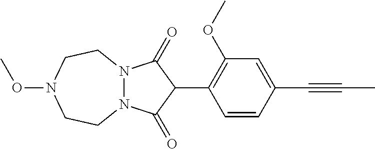 [SMILES: CC#Cc1ccc(C2C(=O)N3CCN(OC)CCN3C2=O)c(OC)c1] | |
| A37 | 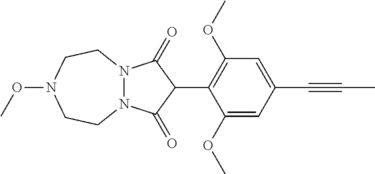 [SMILES: CC#Cc1cc(OC)c(C2C(=O)N3CCN(OC)CCN3C2=O)c(OC)c1] | |
| A38 | 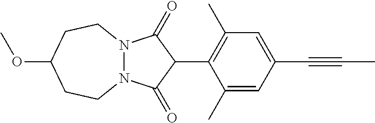 [SMILES: CC#Cc1cc(C)c(C2C(=O)N3CCC(OC)CCN3C2=O)c(C)c1] | |
| A39 | 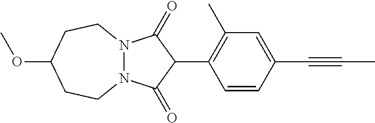 [SMILES: CC#Cc1ccc(C2C(=O)N3CCC(OC)CCN3C2=O)c(C)c1] | |
| A40 | 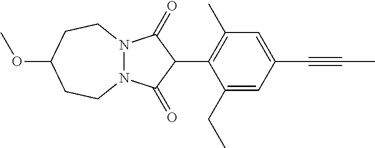 [SMILES: CC#Cc1cc(C)c(C2C(=O)N3CCC(OC)CCN3C2=O)c(CC)c1] | |
| A41 | 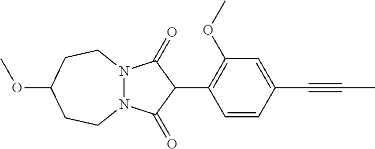 [SMILES: CC#Cc1ccc(C2C(=O)N3CCC(OC)CCN3C2=O)c(OC)c1] | |
| A42 | 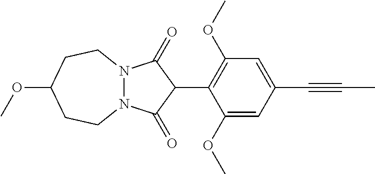 [SMILES: CC#Cc1cc(OC)c(C2C(=O)N3CCC(OC)CCN3C2=O)c(OC)c1] | |
| A43 | 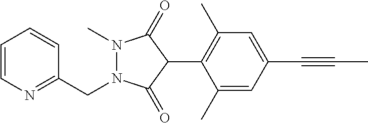 [SMILES: CC#Cc1cc(C)c(C2C(=O)N(C)N(Cc3ccccn3)C2=O)c(C)c1] | |
| A44 | 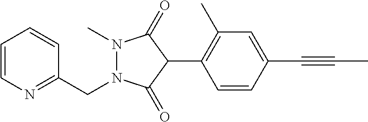 [SMILES: CC#Cc1ccc(C2C(=O)N(C)N(Cc3ccccn3)C2=O)c(C)c1] | |
| A45 | 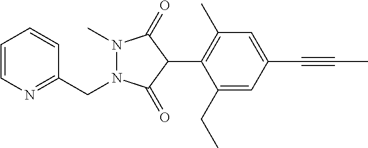 [SMILES: CC#Cc1cc(C)c(C2C(=O)N(C)N(Cc3ccccn3)C2=O)c(CC)c1] | |
| A46 | 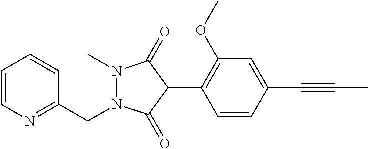 [SMILES: CC#Cc1ccc(C2C(=O)N(C)N(Cc3ccccn3)C2=O)c(OC)c1] | |
| A47 | 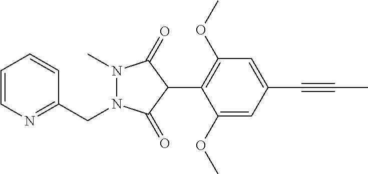 [SMILES: CC#Cc1cc(OC)c(C2C(=O)N(C)N(Cc3ccccn3)C2=O)c(OC)c1] | |
| A48 | 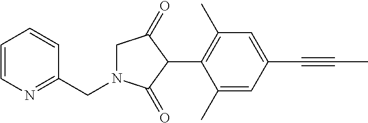 [SMILES: CC#Cc1cc(C)c(C2C(=O)CN(Cc3ccccn3)C2=O)c(C)c1] | 1H NMR (400 MHz, Methanol) δ = 8.51 (d, 1H), 7.83 (dt, 1H), 7.38-7.29 (m, 2H), 7.07 (s, 2H), 4.74 (s, 2H), 4.59 (br. s., 1H), 4.06 (s, 2H), 2.16 (s, 6H), 2.00 (s, 3H). |
| A49 | 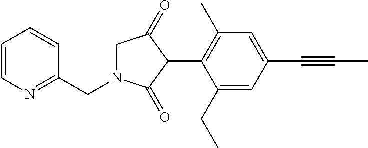 [SMILES: CC#Cc1cc(C)c(C2C(=O)CN(Cc3ccccn3)C2=O)c(CC)c1] | |
| A50 | 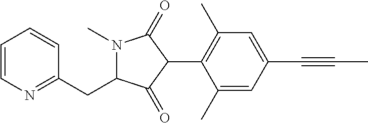 [SMILES: CC#Cc1cc(C)c(C2C(=O)C(Cc3ccccn3)N(C)C2=O)c(C)c1] | 1H NMR (400 MHz, CDCl₃) δ 2.03 (s, 3H) 2.19 (s, 3H) 2.23 (s, 3H) 3.04 (s, 3H) 3.17 (dd, 1H) 3.51 (dd, 1H) 4.20 (dd, 1.7 Hz, 1H) 7.08 (s, 1H) 7.10 (s, 1H) 7.29-7.36 (m, 1H) 7.44 (d, 1H) 7.75-7.93 (m, 1H) 8.47 (d, 1H). |
| A51 | 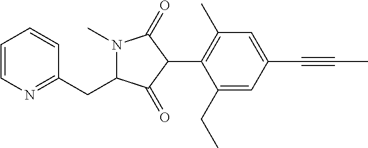 [SMILES: CC#Cc1cc(C)c(C2C(=O)C(Cc3ccccn3)N(C)C2=O)c(CC)c1] | |
| A52 | 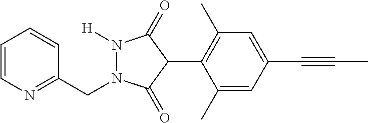 [SMILES: CC#Cc1cc(C)c(C2C(=O)NN(Cc3ccccn3)C2=O)c(C)c1] | |
| A53 | 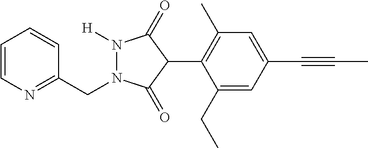 [SMILES: CC#Cc1cc(C)c(C2C(=O)NN(Cc3ccccn3)C2=O)c(CC)c1] | |
| A54 | 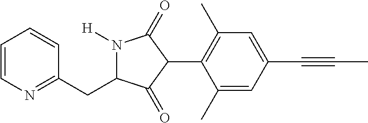 [SMILES: CC#Cc1cc(C)c(C2C(=O)NC(Cc3ccccn3)C2=O)c(C)c1] | |
| A55 | 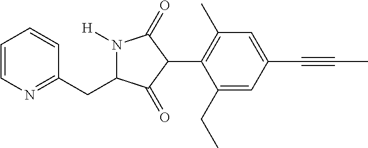 [SMILES: CC#Cc1cc(C)c(C2C(=O)NC(Cc3ccccn3)C2=O)c(CC)c1] | |
| A56 | 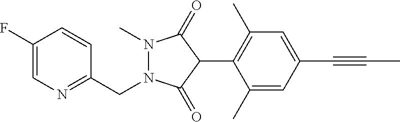 [SMILES: CC#Cc1cc(C)c(C2C(=O)N(C)N(Cc3ccc(F)cn3)C2=O)c(C)c1] | |
| A57 | 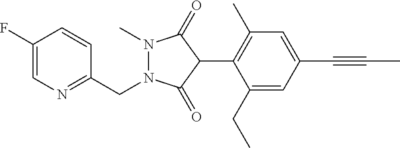 [SMILES: CC#Cc1cc(C)c(C2C(=O)N(C)N(Cc3ccc(F)cn3)C2=O)c(CC)c1] | |
| A58 | 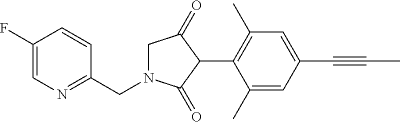 [SMILES: CC#Cc1cc(C)c(C2C(=O)CN(Cc3ccc(F)cn3)C2=O)c(C)c1] | |
| A59 | 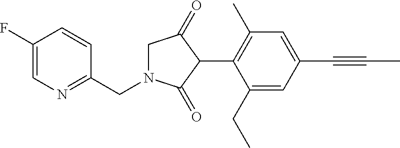 [SMILES: CC#Cc1cc(C)c(C2C(=O)CN(Cc3ccc(F)cn3)C2=O)c(CC)c1] | |
| A60 | 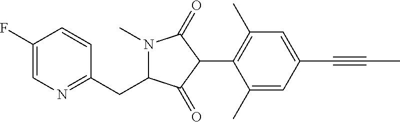 [SMILES: CC#Cc1cc(C)c(C2C(=O)C(Cc3ccc(F)cn3)N(C)C2=O)c(C)c1] | |
| A61 | 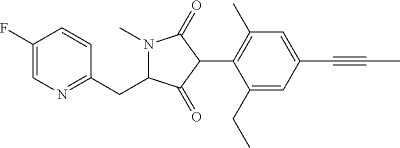 [SMILES: CC#Cc1cc(C)c(C2C(=O)C(Cc3ccc(F)cn3)N(C)C2=O)c(CC)c1] | |
| A62 | 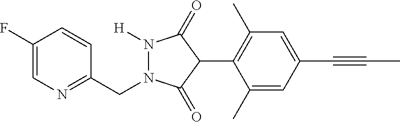 [SMILES: CC#Cc1cc(C)c(C2C(=O)NN(Cc3ccc(F)cn3)C2=O)c(C)c1] | |
| A63 | 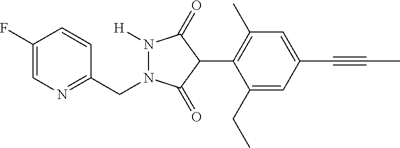 [SMILES: CC#Cc1cc(C)c(C2C(=O)NN(Cc3ccc(F)cn3)C2=O)c(CC)c1] | |
| A64 | 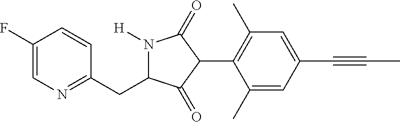 [SMILES: CC#Cc1cc(C)c(C2C(=O)NC(Cc3ccc(F)cn3)C2=O)c(C)c1] | |
| A65 | 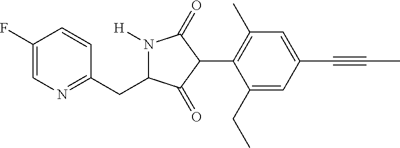 [SMILES: CC#Cc1cc(C)c(C2C(=O)NC(Cc3ccc(F)cn3)C2=O)c(CC)c1] | |
| A66 | 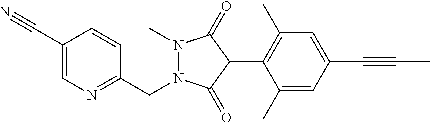 [SMILES: CC#Cc1cc(C)c(C2C(=O)N(C)N(Cc3ccc(C#N)cn3)C2=O)c(C)c1] | |
| A67 | 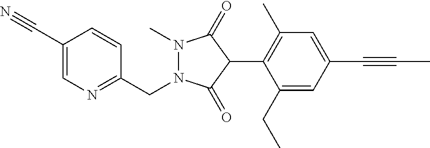 [SMILES: CC#Cc1cc(C)c(C2C(=O)N(C)N(Cc3ccc(C#N)cn3)C2=O)c(CC)c1] | |
| A68 | 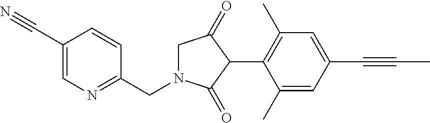 [SMILES: CC#Cc1cc(C)c(C2C(=O)CN(Cc3ccc(C#N)cn3)C2=O)c(C)c1] | |
| A69 | 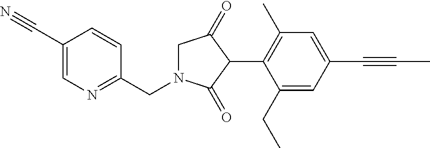 [SMILES: CC#Cc1cc(C)c(C2C(=O)CN(Cc3ccc(C#N)cn3)C2=O)c(CC)c1] | |
| A70 | 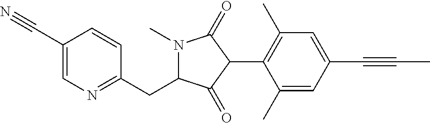 [SMILES: CC#Cc1cc(C)c(C2C(=O)C(Cc3ccc(C#N)cn3)N(C)C2=O)c(C)c1] | |
| A71 | 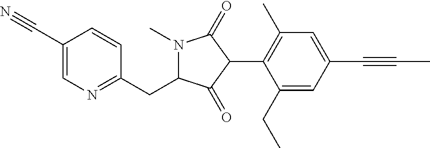 [SMILES: CC#Cc1cc(C)c(C2C(=O)C(Cc3ccc(C#N)cn3)N(C)C2=O)c(CC)c1] | |
| A72 | 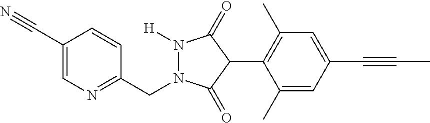 [SMILES: CC#Cc1cc(C)c(C2C(=O)NN(Cc3ccc(C#N)cn3)C2=O)c(C)c1] | |
| A73 | 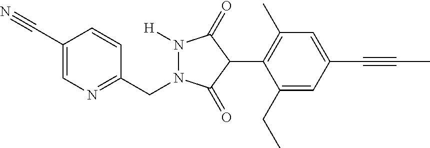 [SMILES: CC#Cc1cc(C)c(C2C(=O)NN(Cc3ccc(C#N)cn3)C2=O)c(CC)c1] | |
| A74 | 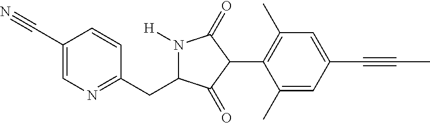 [SMILES: CC#Cc1cc(C)c(C2C(=O)NC(Cc3ccc(C#N)cn3)C2=O)c(C)c1] | |
| A75 | 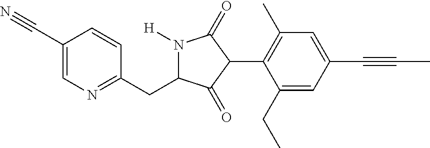 [SMILES: CC#Cc1cc(C)c(C2C(=O)NC(Cc3ccc(C#N)cn3)C2=O)c(CC)c1] | |
| A76 | 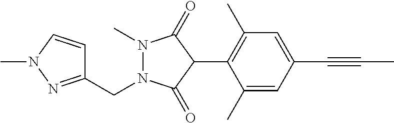 [SMILES: CC#Cc1cc(C)c(C2C(=O)N(C)N(Cc3ccn(C)n3)C2=O)c(C)c1] | |
| A77 | 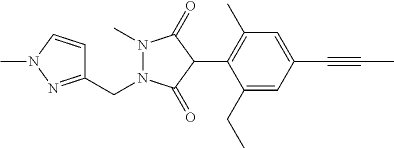 [SMILES: CC#Cc1cc(C)c(C2C(=O)N(C)N(Cc3ccn(C)n3)C2=O)c(CC)c1] | |
| A78 | 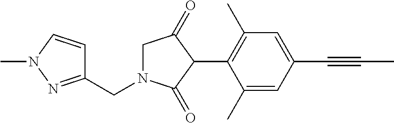 [SMILES: CC#Cc1cc(C)c(C2C(=O)CN(Cc3ccn(C)n3)C2=O)c(C)c1] | |
| A79 | 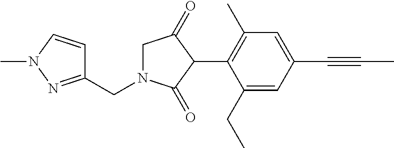 [SMILES: CC#Cc1cc(C)c(C2C(=O)CN(Cc3ccn(C)n3)C2=O)c(CC)c1] | |
| A80 | 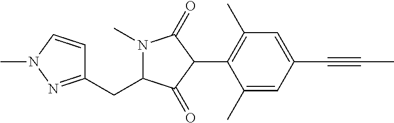 [SMILES: CC#Cc1cc(C)c(C2C(=O)C(Cc3ccn(C)n3)N(C)C2=O)c(C)c1] | |
| A81 | 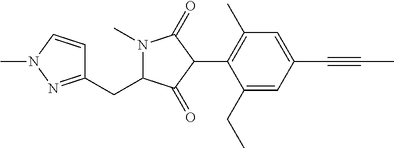 [SMILES: CC#Cc1cc(C)c(C2C(=O)C(Cc3ccn(C)n3)N(C)C2=O)c(CC)c1] | |
| A82 | 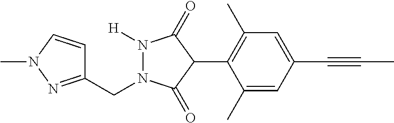 [SMILES: CC#Cc1cc(C)c(C2C(=O)NN(Cc3ccn(C)n3)C2=O)c(C)c1] | |
| A83 | 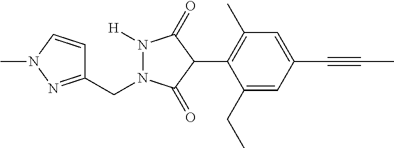 [SMILES: CC#Cc1cc(C)c(C2C(=O)NN(Cc3ccn(C)n3)C2=O)c(CC)c1] | |
| A84 | 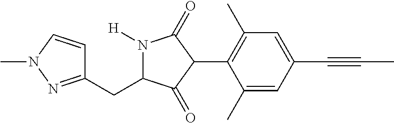 [SMILES: CC#Cc1cc(C)c(C2C(=O)NC(Cc3ccn(C)n3)C2=O)c(C)c1] | |
| A85 | 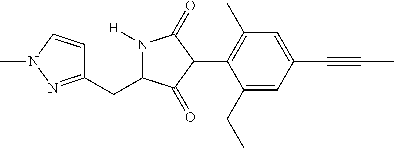 [SMILES: CC#Cc1cc(C)c(C2C(=O)NC(Cc3ccn(C)n3)C2=O)c(CC)c1] | |
| A86 | 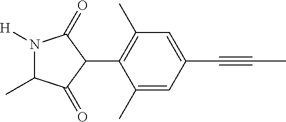 [SMILES: CC#Cc1cc(C)c(C2C(=O)NC(C)C2=O)c(C)c1] | |
| A87 | 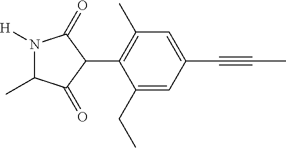 [SMILES: CC#Cc1cc(C)c(C2C(=O)NC(C)C2=O)c(CC)c1] | |
| A88 | 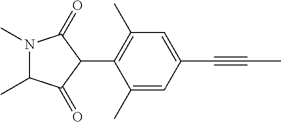 [SMILES: CC#Cc1cc(C)c(C2C(=O)C(C)N(C)C2=O)c(C)c1] | |
| A89 | 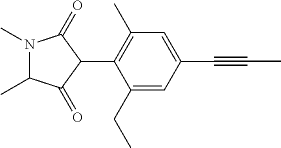 [SMILES: CC#Cc1cc(C)c(C2C(=O)C(C)N(C)C2=O)c(CC)c1] | |
| A90 | 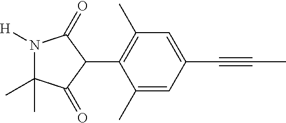 [SMILES: CC#Cc1cc(C)c(C2C(=O)NC(C)(C)C2=O)c(C)c1] | |
| A91 | 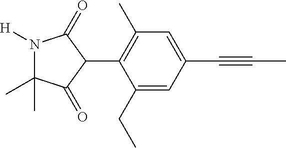 [SMILES: CC#Cc1cc(C)c(C2C(=O)NC(C)(C)C2=O)c(CC)c1] | |
| A92 | 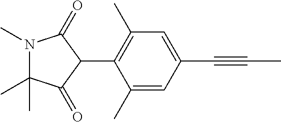 [SMILES: CC#Cc1cc(C)c(C2C(=O)N(C)C(C)(C)C2=O)c(C)c1] | |
| A93 | 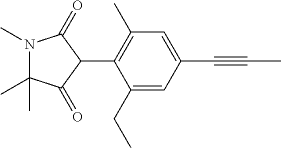 [SMILES: CC#Cc1cc(C)c(C2C(=O)N(C)C(C)(C)C2=O)c(CC)c1] | |
| A94 | 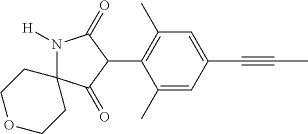 [SMILES: CC#Cc1cc(C)c(C2C(=O)NC3(CCOCC3)C2=O)c(C)c1] | |
| A95 | 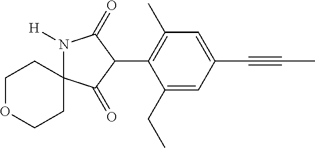 [SMILES: CC#Cc1cc(C)c(C2C(=O)NC3(CCOCC3)C2=O)c(CC)c1] | |

It should be noted that certain compounds of the invention (e.g. in Table T1 hereinabove) may exist as a mixture of isomers, including sometimes atropisomers, under the conditions used to obtain the physicochemical data, in particular ¹H NMR data. Where this has occurred, the characterising ¹H NMR data (where available, e.g. in Table T1 hereinabove) are reported for all isomers present at ambient temperature in the specified solvent. Unless otherwise stated, proton (¹H) NMR spectra disclosed herein (e.g. in Table T1 hereinabove) are recorded at ambient temperature.

TABLE P1

| Com- | | |
|---|---|---|
| pound | | Physicochemical data |
| number | Chemical structure | (or other information) |
| P1 | 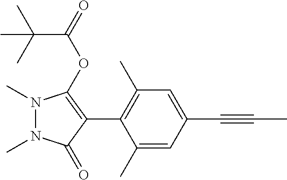 [SMILES: CC#Cc1cc(C)c(-c2c(OC(=O)C(C)(C)C)n(C)n(C)c2=O)c(C)c1] | 1H NMR (CDCl3, 500 MHz) δ 7.07 (s, 2H), 3.42 (s, 3H), 3.17 (s, 3H), 2.16 (s, 6H), 2.02 (s, 3H), 1.08 (s, 9H) |
| P2 | 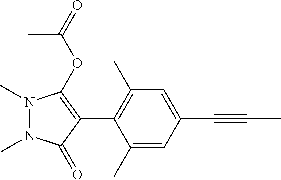 [SMILES: CC#Cc1cc(C)c(-c2c(OC(C)=O)n(C)n(C)c2=O)c(C)c1] | 1H NMR (CDCl3, 500 MHz): δ 7.07 (s, 2H), 3.41 (s, 3H), 3.18 (s, 3H), 2.17 (s, 6H), 2.10 (s, 3H), 2.03 (s, 3H) |
| P3 | 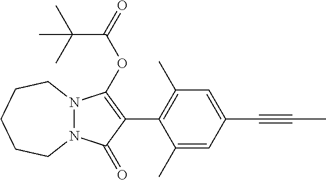 [SMILES: CC#Cc1cc(C)c(-c2c(OC(=O)C(C)(C)C)n3n(c2=O)CCCCC3)c(C)c1] | 1H NMR (CDCl3, 500 MHz) δ 7.06 (s, 2H), 4.05-4.15 (m, 2H), 3.68-3.79 (m, 2H), 2.17 (s, 6H), 2.02 (s, 3H), 1.70-1.87 (m, 6H), 1.05 (s, 9H) |
| P4 | 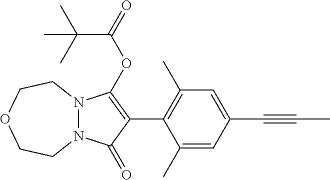 [SMILES: CC#Cc1cc(C)c(-c2c(OC(=O)C(C)(C)C)n3n(c2=O)CCOCC3)c(C)c1] | 1H NMR (CDCl3, 500 MHz) δ 6.88-7.18 (m, 2H), 4.15-4.40 (m, 2H), 3.75-4.01 (m, 6H), 2.15 (s, 6H), 2.01 (s, 3H), 1.04 (s, 9H) |
| P5 | 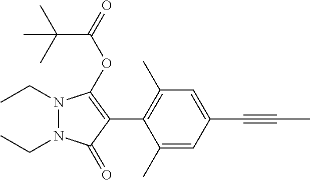 [SMILES: CC#Cc1cc(C)c(-c2c(OC(=O)C(C)(C)C)n(CC)n(CC)c2=O)c(C)c1] | |
| P6 | 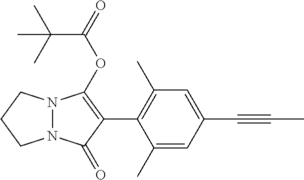 [SMILES: CC#Cc1cc(C)c(-c2c(OC(=O)C(C)(C)C)n3n(c2=O)CCC3)c(C)c1] | |
| P7 | 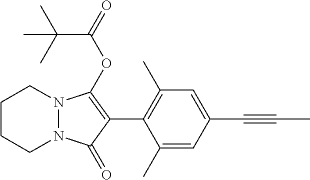 [SMILES: CC#Cc1cc(C)c(-c2c(OC(=O)C(C)(C)C)n3n(c2=O)CCCC3)c(C)c1] | |

The compounds of the following Tables 1 to 47 also illustrate the present invention, and are also particular embodiments of the compounds of formula (I) according to the present invention. In most or many cases, these compounds are thought to be capable of being prepared by methods shown in, or methods similar or analogous to those shown in, the process section and/or the Examples hereinabove, using appropriate starting materials.

Table 1 covers 6 compounds of the following formula

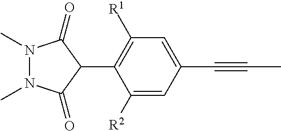

wherein R¹ and R² are as defined in Table 1.

TABLE 1

| Compound | | |
|---|---|---|
| Number | R¹ | R² |
| 1.01 | methyl | methyl |
| 1.02 | methyl | hydrogen |
| 1.03 | methyl | ethyl |
| 1.04 | methoxy | methyl |
| 1.05 | methoxy | hydrogen |
| 1.06 | methoxy | methoxy |

Table 2 covers 6 compounds (compound numbers 2.01 to 2.06) of the following formula

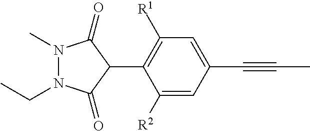

wherein R¹ and R² are as defined in Table 1.

Table 3 covers 6 compounds (compound numbers 3.01 to 3.06) of the following formula

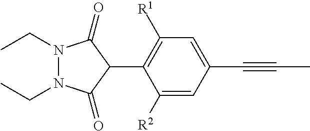

wherein R¹ and R² are as defined in Table 1.

Table 4 covers 6 compounds (compound numbers 4.01 to 4.06) of the following formula

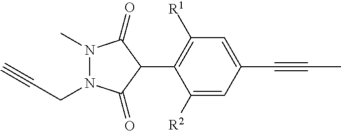

wherein R¹ and R² are as defined in Table 1.

Table 5 covers 6 compounds (compound numbers 5.01 to 5.06) of the following formula

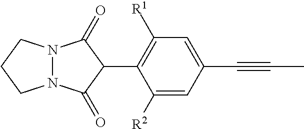

wherein R¹ and R² are as defined in Table 1.

Table 6 covers 6 compounds (compound numbers 6.01 to 6.06) of the following formula

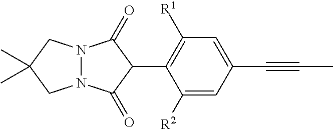

wherein R¹ and R² are as defined in Table 1.

Table 7 covers 6 compounds (compound numbers 7.01 to 7.06) of the following formula

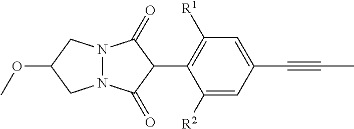

wherein R¹ and R² are as defined in Table 1.

Table 8 covers 6 compounds (compound numbers 8.01 to 8.06) of the following formula

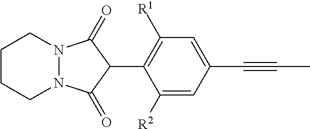

wherein R¹ and R² are as defined in Table 1.

Table 9 covers 6 compounds (compound numbers 9.01 to 9.06) of the following formula

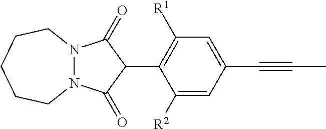

wherein R¹ and R² are as defined in Table 1.

Table 10 covers 6 compounds (compound numbers 10.01 to 10.06) of the following formula

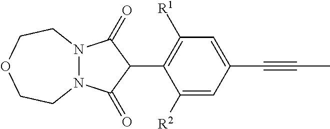

wherein R¹ and R² are as defined in Table 1.

Table 11 covers 6 compounds (compound numbers 11.01 to 11.06) of the following formula

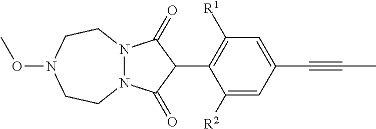

wherein R¹ and R² are as defined in Table 1.

Table 12 covers 6 compounds (compound numbers 12.01 to 12.06) of the following formula

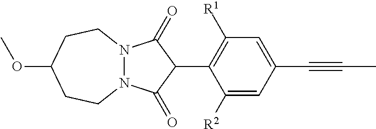

wherein R¹ and R² are as defined in Table 1.

Table 13 covers 6 compounds (compound numbers 13.01 to 13.06) of the following formula

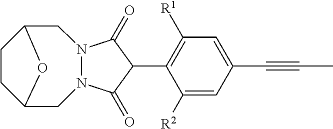

wherein R¹ and R² are as defined in Table 1.

Table 14 covers 6 compounds (compound numbers 14.01 to 14.06) of the following formula

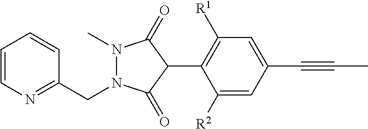

wherein R¹ and R² are as defined in Table 1.

Table 15 covers 6 compounds (compound numbers 15.01 to 15.06) of the following formula

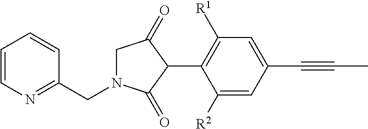

wherein R¹ and R² are as defined in Table 1.

Table 16 covers 6 compounds (compound numbers 16.01 to 16.06) of the following formula

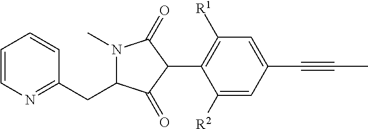

wherein R¹ and R² are as defined in Table 1.

Table 17 covers 6 compounds (compound numbers 17.01 to 17.06) of the following formula

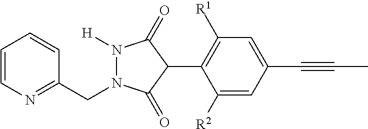

wherein R¹ and R² are as defined in Table 1.

Table 18 covers 6 compounds (compound numbers 18.01 to 18.06) of the following formula

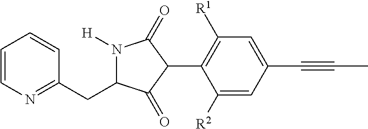

wherein R¹ and R² are as defined in Table 1.

Table 19 covers 6 compounds (compound numbers 19.01 to 19.06) of the following formula

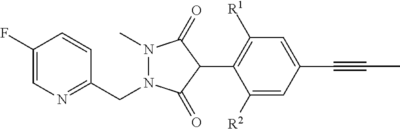

wherein R¹ and R² are as defined in Table 1.

Table 20 covers 6 compounds (compound numbers 20.01 to 20.06) of the following formula

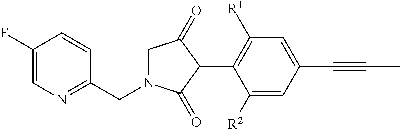

wherein R¹ and R² are as defined in Table 1.

Table 21 covers 6 compounds (compound numbers 21.01 to 21.06) of the following formula

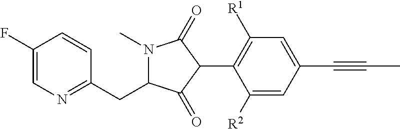

wherein R¹ and R² are as defined in Table 1.

Table 22 covers 6 compounds (compound numbers 22.01 to 22.06) of the following formula

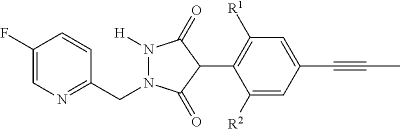

wherein R¹ and R² are as defined in Table 1.

Table 23 covers 6 compounds (compound numbers 23.01 to 23.06) of the following formula

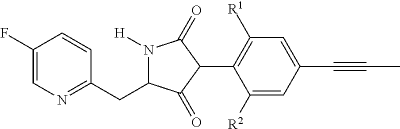

wherein R¹ and R² are as defined in Table 1.

Table 24 covers 6 compounds (compound numbers 24.01 to 24.06) of the following formula

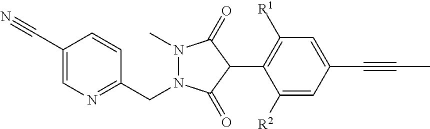

wherein R¹ and R² are as defined in Table 1.

Table 25 covers 6 compounds (compound numbers 25.01 to 25.06) of the following formula

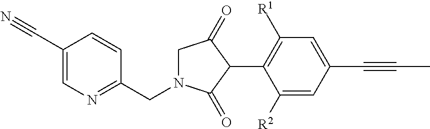

wherein R¹ and R² are as defined in Table 1.

Table 26 covers 6 compounds (compound numbers 26.01 to 26.06) of the following formula

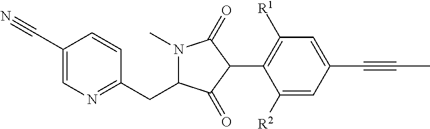

wherein R¹ and R² are as defined in Table 1.

Table 27 covers 6 compounds (compound numbers 27.01 to 27.06) of the following formula

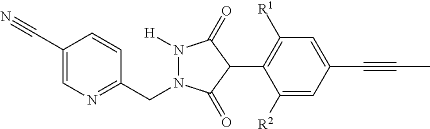

wherein R¹ and R² are as defined in Table 1.

Table 28 covers 6 compounds (compound numbers 28.01 to 28.06) of the following formula

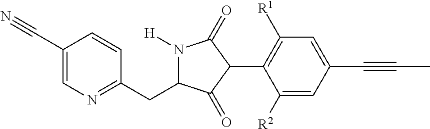

wherein R¹ and R² are as defined in Table 1.

Table 29 covers 6 compounds (compound numbers 29.01 to 29.06) of the following formula

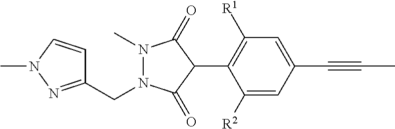

wherein R¹ and R² are as defined in Table 1.

Table 30 covers 6 compounds (compound numbers 30.01 to 30.06) of the following formula

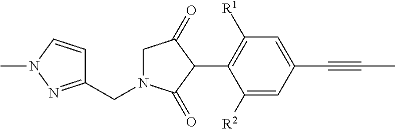

wherein R¹ and R² are as defined in Table 1.

Table 31 covers 6 compounds (compound numbers 31.01 to 31.06) of the following formula

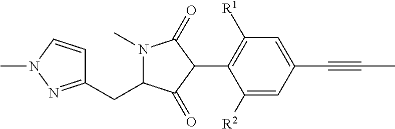

wherein R¹ and R² are as defined in Table 1.

Table 32 covers 6 compounds (compound numbers 32.01 to 32.06) of the following formula

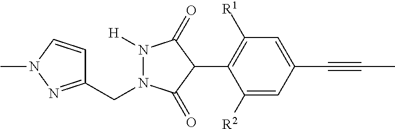

wherein R¹ and R² are as defined in Table 1.

Table 33 covers 6 compounds (compound numbers 33.01 to 33.06) of the following formula

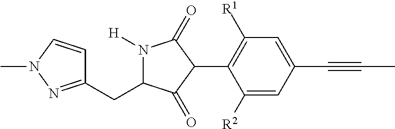

wherein R¹ and R² are as defined in Table 1.

Table 34 covers 6 compounds (compound numbers 34.01 to 34.06) of the following formula

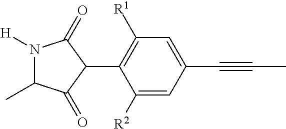

wherein R¹ and R² are as defined in Table 1.

Table 35 covers 6 compounds (compound numbers 35.01 to 35.06) of the following formula

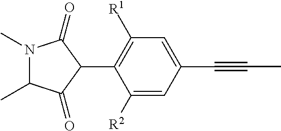

wherein R¹ and R² are as defined in Table 1.

Table 36 covers 6 compounds (compound numbers 36.01 to 36.06) of the following formula

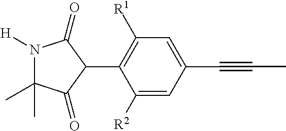

wherein R¹ and R² are as defined in Table 1.

Table 37 covers 6 compounds (compound numbers 37.01 to 37.06) of the following formula

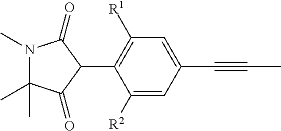

wherein R¹ and R² are as defined in Table 1.

Table 38 covers 6 compounds (compound numbers 38.01 to 38.06) of the following formula

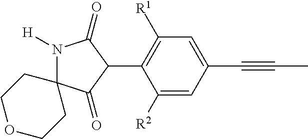

wherein R¹ and R² are as defined in Table 1.

Table 39 covers 6 compounds (compound numbers 39.01 to 39.06) of the following formula

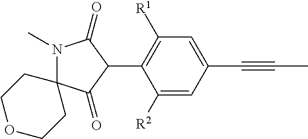

wherein R¹ and R² are as defined in Table 1.

Table 40 covers 6 compounds (compound numbers 40.01 to 40.06) of the following formula

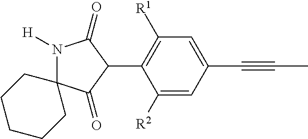

wherein R¹ and R² are as defined in Table 1.

Table 41 covers 6 compounds (compound numbers 41.01 to 41.06) of the following formula

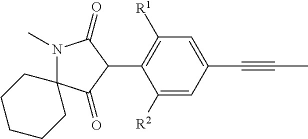

wherein R¹ and R² are as defined in Table 1.

Table 42 covers 6 compounds (compound numbers 42.01 to 42.06) of the following formula

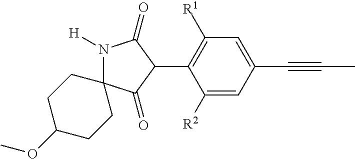

wherein R¹ and R² are as defined in Table 1.

Table 43 covers 6 compounds (compound numbers 43.01 to 43.06) of the following formula

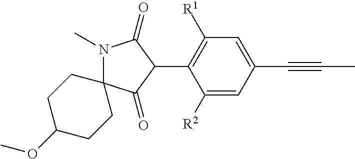

wherein R¹ and R² are as defined in Table 1.

Table 44 covers 6 compounds (compound numbers 44.01 to 44.06) of the following formula

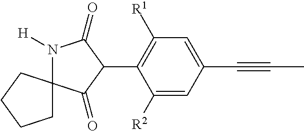

wherein R¹ and R² are as defined in Table 1.

Table 45 covers 6 compounds (compound numbers 45.01 to 45.06) of the following formula

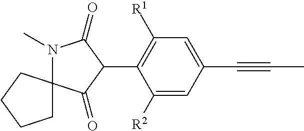

wherein R¹ and R² are as defined in Table 1.

Table 46 covers 6 compounds (compound numbers 46.01 to 46.06) of the following formula

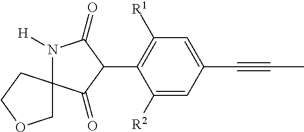

wherein R¹ and R² are as defined in Table 1.

Table 47 covers 6 compounds (compound numbers 47.01 to 47.06) of the following formula

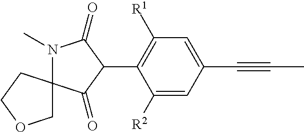

wherein R¹ and R² are as defined in Table 1.

### Biological Examples

### Biological Example 1A

### Test 1A—Glasshouse Assay for Herbicidal Activity

Seeds of a variety of test species are sown in standard soil (usually sandy soil) in pots. After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient (compound) in acetone/water (50:50) solution containing 0.5% Tween 20 (polyoxyethylene sorbitan monolaurate, CAS RN 9005-64-5). The test plants are then grown under controlled conditions in a glasshouse (at 24/16° C., day/night; 14 hours light; 65% humidity) and watered twice daily. 13 days after application of the test compounds, for pre- and post-emergence, the test is evaluated visually for percentage phytotoxicity to each plant (where 100%=total damage to plant; 0%=no damage to plant).

### Biological Example 1A

### Pre-Emergence Herbicidal Activity Results (% Phytotoxicity)

Test plants in the pre-emergence herbicidal activity table below are:

Alopecurus myosuroides (ALOMY), Setaria faberi (SETFA), Echinochloa crus-galli (ECHCG), Amaranthus retroflexus (AMARE), and Abutilon theophasti Medik. (ABUTH, common English name “velvetleaf”). Of these, Alopecurus myosuroides (ALOMY), Setaria faberi (SETFA), and Echinochloa crus-galli (ECHCG) are grassy monocotyledonous weeds.

| | Appli- | | | | | |
|---|---|---|---|---|---|---|
| Com- | cation | | | | | |
| pound | Rate | | | | | |
| Number | (g/ha) | ABUTH | AMARE | SET FA | ALOMY | ECHCG |
| A1 | 250 | 0 | 0 | 30 | 0 | 30 |
| A2 | 250 | NT | 10 | 90 | 80 | 70 |
| A5 | 250 | 0 | 10 | 100 | 90 | 90 |
| A6 | 250 | 30 | 70 | 70 | 50 | 80 |
| A18 | 250 | 50 | 40 | 90 | 100 | 90 |
| A18 | 30 | 40 | 20 | 50 | 60 | 60 |
| A23 | 250 | 10 | 80 | 50 | 90 | 90 |
| A28 | 250 | 10 | 30 | 100 | 100 | 100 |
| A28 | 30 | 10 | 20 | 50 | 70 | 80 |
| P1 | 250 | 10 | 10 | 100 | 100 | 100 |
| P2 | 250 | 0 | 70 | 90 | 90 | 100 |
| P3 | 250 | 10 | 20 | 70 | 20 | 20 |
| P4 | 250 | 80 | 100 | 100 | 100 | 100 |

Note:
NT = not tested

Test plants in the pre-emergence herbicidal activity table below are:

Lolium perenne (LOLPE), Triticum aestivum (TRZAW, winter wheat), Alopecurus myosuroides (ALOMY), Echinochloa crus-galli (ECHCG), and Avena fatua (AVEFA). All these are grassy monocotyledonous weeds and/or (for wheat) grassy crops.

| | Appli- | | | | | |
|---|---|---|---|---|---|---|
| Com- | cation | | | | | |
| pound | Rate | | | | | |
| Number | (g/ha) | LOLPE | TRZAW | ALOMY | ECHCG | AVEFA |
| A48 | 250 | 80 | 40 | 90 | 80 | 40 |
| A50 | 250 | 100 | 90 | 90 | 90 | 70 |
| A50 | 62.5 | 70 | 70 | 70 | 70 | 60 |
| Com- | 250 | 50 | 0 | 0 | 10 | 0 |
| pound A* | | | | | | |

*Compound A is a comparative example, outside of the scope of the present invention, and differs from compound A48 in that the p-propynyl group is substituted with ethynyl.

### Biological Example 1A

### Post-Emergence Herbicidal Activity Results (% Phytotoxicity)

Test plants in the post-emergence herbicidal activity table below are:

Alopecurus myosuroides (ALOMY), Setaria faberi (SETFA), Echinochloa crus-galli (ECHCG), Amaranthus retroflexus (AMARE), and Abutilon theophasti Medik. (ABUTH, common English name “velvetleaf”). Of these, Alopecurus myosuroides (ALOMY), Setaria faberi (SETFA), and Echinochloa crus-galli (ECHCG) are grassy monocotyledonous weeds.

| | Appli- | | | | | |
|---|---|---|---|---|---|---|
| Com- | cation | | | | | |
| pound | Rate | | | | | |
| Number | (g/ha) | ABUTH | AMARE | SET FA | ALOMY | ECHCG |
| A1 | 250 | 10 | 0 | 40 | 0 | 40 |
| A2 | 250 | 10 | 100 | 80 | 70 | 90 |
| A5 | 250 | 0 | 0 | 90 | 90 | 100 |
| A5 | 30 | 0 | 0 | 80 | 70 | 90 |
| A6 | 250 | 30 | 30 | 80 | 80 | 80 |
| A6 | 30 | 30 | 0 | 70 | 20 | 40 |
| A18 | 250 | 80 | 80 | 90 | 80 | 100 |
| A18 | 30 | 70 | 40 | 70 | 50 | 20 |
| A23 | 250 | 60 | 30 | 80 | 80 | 80 |
| A28 | 250 | 70 | 30 | 100 | 90 | 100 |
| A28 | 30 | 60 | 10 | 70 | 60 | 80 |
| P1 | 250 | 70 | 20 | 90 | 90 | 100 |
| P1 | 30 | 30 | 0 | 70 | 80 | 80 |
| P2 | 250 | 60 | 30 | 90 | 90 | 100 |
| P2 | 30 | 10 | 0 | 40 | 40 | 80 |
| P3 | 250 | 0 | NT | 70 | 50 | 100 |
| P3 | 30 | 10 | 0 | 20 | 40 | 60 |
| P4 | 250 | 70 | 10 | 100 | 90 | 100 |
| P4 | 30 | 70 | 0 | 70 | 70 | 100 |

Note:
NT = not tested

Test plants in the post-emergence herbicidal activity table below are:

Lolium perenne (LOLPE), Triticum aestivum (TRZAW, winter wheat, variety ‘Hereward’), Alopecurus myosuroides (ALOMY), Echinochloa crus-galli (ECHCG), and Avena fatua (AVEFA). All these are grassy monocotyledonous weeds and/or (for wheat) grassy crops.

| | Appli- | | | | | |
|---|---|---|---|---|---|---|
| Com- | cation | | | | | |
| pound | Rate | | | | | |
| Number | (g/ha) | LOLPE | TRZAW | ALOMY | ECHCG | AVEFA |
| A48 | 250 | 70 | 30 | 90 | 70 | 20 |
| A50 | 250 | 100 | 80 | 100 | 100 | 100 |
| A50 | 62.5 | 70 | 70 | 90 | 100 | 70 |
| A50 | 15.625 | 40 | 30 | 30 | 80 | 50 |
| Com- | 250 | 0 | 0 | 0 | 10 | 0 |
| pound A* | | | | | | |

*Compound A is a comparative example, outside of the scope of the present invention, and differs from compound A48 in that the p-propynyl group is substituted with ethynyl.

### Biological Example 1B

### Test 1B—Glasshouse Assay for Post-Emergence Herbicidal Activity (Phytotoxicity), on Winter Wheat, and on Winter Wheat Whose Seed has been Treated with Cloquintocet-Mexyl

Seeds of the Winter Wheat variety ‘Hereward’ were sown in standard soil (usually sandy soil) in pots. After 8 days cultivation under controlled conditions in a glasshouse (at 24/16° C., day/night; 14 hours light; 65% humidity), which is post-emergence, the plants were sprayed with an aqueous spray solution derived from the formulation of the technical active ingredient (compound) in acetone/water (50:50) solution containing 0.5% Tween 20 (polyoxyethylene sorbitan monolaurate, CAS RN 9005-64-5).

Seeds of the Winter Wheat variety ‘Hereward’ were seed treated with a wettable powder formulation of the cereal herbicide safener, cloquintocet-mexyl, at a rate of 0.5 grams per kilogram of dry seed, prior to the initiation of glasshouse testing. Three seeds were sown per 1.5 inch plastic pot into a standard soil (usually sandy soil) at a depth of 1 cm, prior to application of the test compounds and were watered and grown under controlled conditions in a glasshouse (at 24/16° C., day/night; 14 hours light; 65% humidity). After 8 days cultivation under these controlled conditions, the plants were sprayed post-emergence with an aqueous spray solution derived from the formulation of the technical active ingredient (compound) in acetone/water (50:50) solution containing 0.5% Tween 20 (polyoxyethylene sorbitan monolaurate, CAS RN 9005-64-5).

The test plants were then grown on in a glasshouse under controlled conditions (at 24/16° C., day/night; 14 hours light; 65% humidity) and watered twice daily. 13 days after application of the test compounds, the test was evaluated visually for percentage phytotoxicity to each plant (where 100%=total damage to plant; 0%=no damage to plant).

### Biological Example 1B

### Post-Emergence Herbicidal Activity Results (% Phytotoxicity)

| | | | Cloquintocet-mexyl |
|---|---|---|---|
| | Application | Winter Wheat | seed-treated Winter |
| Compound | Rate | (variety | Wheat (variety |
| Number | (g/ha) | ‘Hereward’) | ‘Hereward’) |
| A50 | 250 | 80 | 60 |
| A50 | 62.5 | 70 | 40 |
| A50 | 15.625 | 30 | 0 |

### Biological Example 2

### Test 2—Glasshouse Assay for Herbicidal Activity

Seeds of a variety of monocotyledonous and dicotyledonous test plants are sown in standard soil in pots. The plants are cultivated for one day (for pre-emergence) or for about 12 days (range=10-13 days) (for post-emergence) under controlled conditions in a glasshouse (warm climate species at 24/18° C., cool climate species at 20/16° C., both at day/night; 16 hours light; 65% humidity).

An “instant formulation”, known as the “IF50”, containing 50 g/litre (i.e. 5% w/v) of the “technical” (i.e. unformulated) active ingredient is prepared by dissolving the active ingredient in a mixture of organic solvents and emulsifier, details of which are provided in the Table below. This IF50 is then mixed with a small, variable amount of acetone to aid dissolution, before addition of a 0.2% v/v aqueous solution of the adjuvant X-77 (which is a mixture of alkyl aryl polyoxyethylene glycols and free fatty acids in isopropanol, CAS Registry number 11097-66-8), as the aqueous diluent, to form an aqueous spray solution which contains a predetermined concentration of the active ingredient (which varies depending on the application rate of the active ingredient to the plants) and 0.2% v/v of the adjuvant X-77. This aqueous spray solution is then sprayed onto the plants, after one day's cultivation (for pre-emergence) or after about 12 days' cultivation (for post-emergence).

Table: Composition of the mixture of organic solvents and emulsifier to be used as a base for the instant formulation (IF50).

| | | | CAS | |
|---|---|---|---|---|
| | | Chemical | Registry | Amount/ |
| Component | Supplier | description | number | % w/w |
| Emulsogen | Clariant | castor oil | 61791-12-6 | 11.12 |
| EL360 ™ | | ethoxylate | | |
| | | (as emulsifier) | | |
| N-meth- | widely | 1-methyl-2- | 872-50-4 | 44.44 |
| ylpyrrolidone | available | pyrrolidone | | |
| Dowanol DPM ™ | Dow | dipropylene | 34590-94-8 | 44.44 |
| glycol ether | | glycol | | |
| | | monomethyl | | |
| | | ether | | |

The test plants are then grown on, in a glasshouse (greenhouse) under controlled conditions (at either 24/18° C. or 20/16° C. (day/night) as mentioned above; 16 hours light; 65% humidity) and are watered twice daily. Either 15 days after application of the test compound (15 DAA) (for post-emergence), or 20 days after application of the test compound (20 DAA) (for pre-emergence), the test plants are evaluated visually, and an assessed percentage phytotoxicity score is given for each compound application on each plant species (where 100%=total damage to plant; 0%=no damage to plant).

Some of the typical test plants are as follows:

Cool climate crop plants: Triticum aestivum (TRZAW, winter wheat), Brassica napus (BRSNN, rape, also called oilseed rape or rapeseed), Beta vulgaris (BEAVA, sugar beet).
Warm climate crop plants: Glycine max (GLXMA, soybean).
Cool climate (“cool season”) grassy monocotyledonous weeds: Alopecurus myosuroides (ALOMY), Avena fatua (AVEFA), Lolium perenne (LOLPE).
Warm climate (“warm season”) grassy monocotyledonous weeds: Setaria faberi (SETFA), SORVU (Sorghum bicolor (L.) Moench ssp. Bicolor, or Sorghum vulgare Pers.), Digitaria sanguinalis (DIGSA), Echinochloa crus-galli (ECHCG), and Brachiaria plantaginea (BRAPL).

### Biological Example 3

### Table of Post-Emergence Herbicidal Activity (Percentage Phytotoxicity)

| | Appli- | | | | | | |
|---|---|---|---|---|---|---|---|
| Com- | cation | | | | | | |
| pound | Rate | | | | | | |
| Number | (g/ha) | TRZAW | GLXMA | BRSNN | BEAVA | ALOMY | LOLPE |
| A1 | 125 | 80 | 70 | 70 | 10 | 70 | 80 |
| A1 | 30 | 30 | 30 | 70 | 0 | 10 | 30 |
| A23 | 125 | 50 | 70 | 50 | 40 | 50 | 40 |
| A23 | 30 | 0 | 0 | 30 | 10 | 0 | 0 |
| P1 | 125 | 70 | 40 | 80 | 40 | 70 | 80 |
| P1 | 30 | 0 | 30 | 80 | 10 | 60 | 50 |
| P1 | 8 | 0 | 10 | 0 | 30 | 40 | 30 |
| P4 | 125 | 80 | 70 | 80 | 50 | 70 | 50 |
| P4 | 30 | 40 | 60 | 80 | 40 | 60 | 40 |
| P4 | 8 | 10 | 50 | 70 | 30 | 20 | 30 |

| Com- | | | | | | | |
|---|---|---|---|---|---|---|---|
| pound | | | | | | | |
| Number | POAAN | SETFA | SORVU | DIGSA | ECHCG | BRAPL | ZEAMX |
| A1 | 80 | NT | 80 | 90 | 90 | 100 | 100 |
| A1 | 50 | NT | 70 | 80 | 70 | 70 | 90 |
| A23 | 70 | 70 | 70 | 70 | 20 | 10 | 30 |
| A23 | 10 | 0 | 50 | 30 | 0 | 0 | 0 |
| P1 | 70 | 100 | 80 | 100 | 100 | 100 | 100 |
| P1 | 0 | 90 | 60 | 90 | 70 | 70 | 70 |
| P1 | 0 | 20 | 50 | 80 | 20 | 60 | 60 |
| P4 | 90 | 90 | 100 | 90 | 100 | 100 | 100 |
| P4 | 70 | 80 | 80 | 90 | 90 | 90 | 90 |
| P4 | 50 | 70 | 70 | 80 | 60 | 90 | 30 |

1. A compound of formula (I): 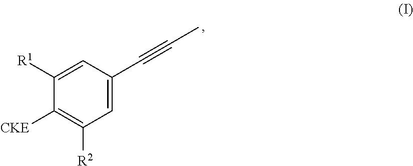
wherein CKE is of sub-formula (A) and/or (B): 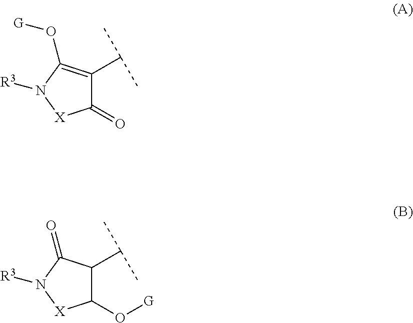
wherein:
R¹ is methyl or methoxy;
R² is hydrogen, methyl, ethyl, n-propyl, n-butyl, cyclopropyl, ethynyl, cyano, C₁-C₃alkoxy, C₁-C₂fluoroalkoxy, (difluoro)vinyloxy, C₁-C₂alkoxy-C₁-C₃alkoxy-, or C₁fluoroalkoxy-C₁-C₃alkoxy-;
X is NR⁴ or CR⁵R⁶;
wherein:
R³, R⁴, and R⁵, independently of each other, are:
hydrogen, C₁-C₄alkyl, C₁-C₂fluoroalkyl, C₁-C₃alkoxyC₁-C₃alkyl, C₁-C₃alkylthioC₁-C₃alkyl, C₁-C₄alkoxy, R⁹—C≡C—C(R⁷)(R⁸)—, (R¹¹)(R¹²)C═C(R¹⁰)—C(R⁷)(R⁸)—, Het-CH₂—, or Het; or
benzyl optionally substituted on its phenyl ring by 1 or 2 substituents independently being halogen, C₁-C₂alkyl, C₁fluoroalkyl, C₁-C₂alkoxy, C₁fluoroalkoxy, —C≡C—R¹³, —C(R¹⁴)═C(R¹⁵)(R¹⁶), —C(O)Me, —C(O)—C₁fluoroalkyl, —S(O)ₙ₄-Me, —S(O)ₙ₄—C₁fluoroalkyl, —NR¹⁹R²⁰, or cyano; or
phenyl optionally substituted by 1 or 2 substituents independently being halogen (preferably fluorine, chlorine or bromine), C₁-C₂alkyl, C₁fluoroalkyl, —C≡C—R¹³, —C(R¹⁴)═C(R¹⁵)(R¹⁶), C₁-C₂alkoxy, C₁fluoroalkoxy, —C(O)Me, —C(O)—C₁fluoroalkyl, —S(O)ₙ₅-Me, —S(O)ₙ₅—C₁fluoroalkyl, —NR¹⁹R²⁰, or cyano;
provided that when X is NR⁴, then at least one of R³ and R⁴ is hydrogen or C₁-C₃alkyl; and provided that when X is CR⁵R⁶, then at least one of R³ and R⁵ is hydrogen or C₁-C₃alkyl;
or R³ and R⁴ are taken together, and/or R³ and R⁵ are taken together, to form a chain which is (a), (b), (c), (d), (e), (f) or (g), as shown below:
—C(R²¹)(R²²)—C(R²³)(R²⁴)—Y—C(R²⁵)(R²⁶)—C(R²⁷)(R²⁸)— (a)
—C(R²¹)(R²²)—Y—C(R^{21A})(R^{22A})—C(R^{21A})(R^{22A})—C(R²⁷)(R²⁸)— (b)
—CH(R²⁹)—Y—C(R^{21A})(R^{22A})—CH(R³²)— (c)
—CH(R²⁹)—Y—CH(R³²)— (d)
—C(R²¹)(R²²)—C(R²³)(R²⁴)—C(R³⁰)(R³¹)—C(R²⁵)(R²⁶)—C(R²⁷)(R²⁸)— (e)
—C(R²¹)(R²²)—C(R^{21A})(R^{22A})—C(R³⁰)(R³¹)—C(R²⁷)(R²⁸)— (f)
—CH(R²⁹)—C(R³⁰)(R³¹)—CH(R³²)— (g)
wherein Y is O or NR⁴⁰;
and R⁶ is hydrogen or C₁-C₃alkyl;
or R⁵ and R⁶ taken together are —(CH₂)ₙ₁— or —(CH₂)ₙ₂—X¹—(CH₂)ₙ₃—, in which case R³ is as defined herein provided that R³ and R⁵ are not taken together;
wherein X¹ is O, C(H)(C₁-C₂alkyl), C(C₁-C₂alkyl)₂ or C(H)(C₁-C₂alkoxy);
n1 is 2, 3, 4 or 5; and
n2 and n3 are independently 1, 2 or 3 provided that n2+n3 is 2, 3 or 4;
and wherein:
Het is a heteroaryl, attached at a ring-carbon, which is optionally substituted by 1, 2 or 3 ring-carbon substituents independently being halogen, C₁-C₃alkyl, C₁-C₂fluoroalkyl, —C≡C—R¹³, —C(R¹⁴)═C(R¹⁵)(R¹⁶), C₁-C₃alkoxy, C₁-C₂fluoroalkoxy, cyclopropyloxy, CH₂═CH—CH₂—O—, HC≡C—CH₂—O—, —C(O)—R¹⁷, —S(O)ₙ₁₈— R¹⁸, —NR¹⁹R²⁰, cyano or nitro; provided that any non-fluorine halogen, alkoxy, fluoroalkoxy, cyclopropyloxy, CH₂═CH—CH₂—O— or HC≡C—CH₂—O— is not substituted at any ring-carbon bonded directly to a ring-nitrogen of the heteroaryl;
and/or, when Het is a 5-membered heteroaryl ring containing a ring-nitrogen atom not partaking in a C═N ring double bond, the heteroaryl is optionally substituted on the ring-nitrogen atom not partaking in a C═N ring double bond by one C₁-C₃alkyl, C₁-C₂fluoroalkyl, C₁-C₂fluoroalkyl-C(O)— or C₁-C₂alkyl-S(O)₂— substituent;
and wherein:
R⁷ and R⁸, independently of each other, are hydrogen or methyl;
R⁹ is hydrogen, methyl, ethyl or chlorine; and
R¹⁰, R¹¹ and R¹², independently of each other, are hydrogen, methyl or ethyl, provided that R¹⁰, R¹¹ and R¹² in total contain no more than three carbon atoms;
R¹³ is hydrogen, methyl, C₁fluoroalkyl, fluorine or chlorine;
R¹⁴, R¹⁵ and R¹⁶ independently are hydrogen, methyl, C₁fluoroalkyl, fluorine or chlorine;
provided that R¹⁴, R¹⁵ and R¹⁶ in total contain no more than one carbon atom, and R¹⁴, R¹⁵ and R¹⁶ in total comprise no more than one chlorine;
and
n4 is 0, 1 or 2;
n5 is 0, 1 or 2;
R¹⁷ and R¹⁸ independently are C₁-C₃alkyl, C₁fluoroalkyl, or —NR^{19A}R^{20A};
n18 is 0, 1 or 2;
R¹⁹ is —C(O)—C₁-C₂alkyl, —C(O)—C₁fluoroalkyl, —S(O)₂—C₁-C₂alkyl, —S(O)₂—C₁fluoroalkyl, C₁-C₂alkyl, or C₁fluoroalkyl;
R²⁰ and R^{20A} independently are hydrogen, methyl or C₁fluoroalkyl; and
R^{19A} is hydrogen, C₁-C₂alkyl or C₁fluoroalkyl;
and wherein:
R²¹, R²², R²⁷ and R²⁸ are independently hydrogen or methyl; provided that two, three or all of R²¹, R²², R²⁷ and R²⁸ are hydrogen; and
R^{21A} and R^{22A} are independently hydrogen or methyl; and
R²³, R²⁴, R²⁵, and R²⁶ are independently hydrogen, methyl, ethyl or methoxymethyl; provided that two, three or all of R²³, R²⁴, R²⁵ and R²⁶ are hydrogen;
or R²³ and R²⁵ are taken together and form a —CH₂— or —CH₂CH₂— bridge, and R²⁴ and R²⁶ are hydrogen; and
R²⁹ and R³² are independently hydrogen or methyl; and
R³⁰ is hydrogen, C₁-C₃alkyl, C₁-C₂fluoroalkyl, C₁-C₃alkoxy, C₁-C₃fluoroalkoxy, C₁-C₃alkyl-S(O)ₙ₃₀—, C₁-C₂alkoxyC₁-C₂alkyl, or C₁-C₂alkylthioC₁-C₂alkyl;
wherein n30 is 0, 1 or 2; and
R³¹ is hydrogen, methyl or ethyl;
or R³⁰ and R³¹ taken together are oxo (═O), ═N—O—H, or ═N—O—(C₁-C₃alkyl);
provided that, in all cases wherein R³ and R⁴ are taken together, and/or R³ and R⁵ are taken together, to form a chain which is (a), (b), (c), (d), (e), (f) or (g), then the chain contains a total of no more than three substituents (preferably a total of none, one or two substituents) on carbon in the ring which is formed by NR³, X, and the chain (a), (b), (c), (d), (e), (f) or (g);
wherein if a bridge is formed by R²³ and R²⁵ being taken together then this bridge is counted as two ring-carbon-substituents;
and wherein R⁴⁰ is C₁-C₃alkoxy, C₁-C₂fluoroalkoxy, cyclopropyloxy, CH₂═CH—CH₂—O—, HC≡C—CH₂—O—, N≡C—CH₂O—, CH₃OCH₂O—, CH₃OCH₂CH₂O—, CH₃CH₂OCH₂O—, C₁-C₃alkyl or C₁fluoroalkyl;
and wherein:
G is hydrogen; an agriculturally acceptable metal, or an agriculturally acceptable sulfonium or ammonium group; or
G is —C(X^{a})—R^{a}, —C(X^{b})—X^{c}—R^{b}, —C(X^{d})—N(R^{c})—R^{d}, —SO₂—R^{e}, —P(X^{e})(R^{f})—R^{g}, —CH₂—X^{f}—R^{b}; or phenyl-CH₂— or phenyl-CH(C₁-C₂alkyl)- (in each of which the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or heteroaryl-CH₂— or heteroaryl-CH(C₁-C₂alkyl)- (in each of which the heteroaryl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro), or phenyl-C(O)—CH₂— (wherein the phenyl is optionally substituted by 1, 2 or 3 of, independently, C₁-C₂alkyl, C₁fluoroalkyl, C₁-C₂alkoxy, C₁fluoroalkoxy, fluorine, chlorine, bromine, cyano or nitro); or C₁-C₆alkoxy-C(O)—CH₂—, C₁-C₆alkoxy-C(O)—CH═CH—, C₂-C₇alken-1-yl-CH₂—, C₂-C₇alken-1-yl-CH(C₁-C₂alkyl)-, C₂-C₄fluoroalken-1-yl-CH₂—, C₂-C₇alkyn-1-yl-CH₂—, or C₂-C₇alkyn-1-yl-CH(C₁-C₂alkyl)-;
wherein X^{a}, X^{b}, X^{c}, X^{d}, X^{e} and X^{f} are independently of each other oxygen or sulfur; and
wherein
R^{a} is H, C₂-C₂₁alkenyl, C₂-C₁₈alkynyl, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro;
R^{b} is C₁-C₁₈alkyl, C₃-C₁₈alkenyl, C₃-C₁₈alkynyl, C₂-C₁₀fluoroalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroarylC₁-C₅alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkyl-thio, C₁-C₃alkylsulfinyl, C₁-C₃alkylsulfonyl, halogen, cyano, or nitro), C₃-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; and
R^{c} and R^{d} are each independently of each other hydrogen, C₁-C₁₀alkyl, C₃-C₁₀alkenyl, C₃-C₁₀alkynyl, C₂-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₅alkoxy(C₁-C₅)alkyl, C₃-C₅alkenyloxy(C₁-C₅)alkyl, C₃-C₅alkynyloxy(C₁-C₅)alkyl, C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃alkyl, C₁-C₃fluoroalkyl, C₃alkoxy, C₁-C₃fluoroalkoxy, halogen, cyano or nitro; diheteroarylamino or diheteroarylamino substituted by 1, 2 or 3 of, independently, C₁-C₃alkyl, C₁-C₃fluoroalkyl, C₁-C₃alkoxy, C₃fluoroalkoxy, halogen, cyano or nitro; phenylamino or phenylamino substituted by 1, 2 or 3 of, independently, C₁-C₃alkyl, C₁-C₃fluoroalkyl, C₁-C₃alkoxy, C₁-C₃fluoroalkoxy, halogen, cyano or by nitro; diphenylamino or diphenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or C₃-C₇cycloalkylamino, di(C₃-C₇cycloalkyl)amino or C₃-C₇cycloalkoxy;
or R^{c} and R^{d}, together with the nitrogen to which they are bonded, to form an unsubstituted 4, 5, 6 or 7 (e.g. 5 or 6) membered ring, optionally containing one heteroatom selected from O or S; and
R^{e} is C₁-C₁₀alkyl, C₂-C₁₀alkenyl, C₂-C₁₀alkynyl, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₅alkylaminocarbonyl(C₁-C₅)alkyl, C₂-C₈dialkylaminocarbonyl(C₁-C₅)alkyl, C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; diheteroarylamino or diheteroarylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; phenylamino or phenylamino substituted by 1, 2 or 3 of, independently, C₁-C₃alkyl, C₁-C₃fluoroalkyl, C₃alkoxy, C₁-C₃fluoroalkoxy, halogen, cyano or nitro; diphenylamino or diphenylamino substituted by 1, 2 or 3 of, independently, C₁-C₃alkyl, C₁-C₃fluoroalkyl, C₁-C₃alkoxy, C₃fluoroalkoxy, halogen, cyano or nitro; or C₃-C₇cycloalkylamino, di(C₃-C₇cycloalkyl)amino, C₃-C₇cycloalkoxy, C₁-C₁₀alkoxy, C₁-C₁₀fluoroalkoxy, C₁-C₅alkylamino or di(C₁-C₄alkyl)amino;
R^{f} and R^{g} are each independently of each other C₁-C₁₀alkyl, C₂-C₁₀alkenyl, C₂-C₁₀alkynyl, C₁-C₁₀alkoxy, C₁-C₁₀fluoroalkyl, C₁-C₁₀cyanoalkyl, C₁-C₁₀nitroalkyl, C₁-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₁-C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₅alkoxycarbonyl(C₁-C₅)alkyl, aminocarbonyl(C₁-C₅)alkyl, C₁-C₅alkylaminocarbonyl(C₁-C₅)alkyl, C₂-C₈dialkylaminocarbonyl(C₁-C₅)alkyl, C₁-C₅alkylcarbonylamino(C₁-C₅)alkyl, N—(C₁-C₅)alkylcarbonyl-N—(C₂-C₅)alkylaminoalkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₃fluoroalkyl, C₁-C₃alkoxy, C₁-C₃fluoroalkoxy, C₁-C₃alkylthio, C₁-C₃alkylsulfinyl, C₃alkylsulfonyl, halogen, cyano, or nitro), heteroaryl(C₁-C₅)alkyl (wherein the heteroaryl is optionally substituted by 1, 2 or 3 of, independently, C₁-C₃alkyl, C₁-C₃fluoroalkyl, C₃alkoxy, C₁-C₃fluoroalkoxy, C₁-C₃alkylthio, C₁-C₃alkylsulfinyl, C₁-C₃alkylsulfonyl, halogen, cyano, or nitro), C₂-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃ alkyl, C₁-C₃fluoroalkyl, C₁-C₃alkoxy, C₁-C₃fluoroalkoxy, halogen, cyano or nitro; heteroarylamino or heteroarylamino substituted by 1, 2 or 3 of, independently, C₁-C₃ alkyl, C₁-C₃fluoroalkyl, C₃alkoxy, C₁-C₃fluoroalkoxy, halogen, cyano or nitro; diheteroarylamino or diheteroarylamino substituted by 1, 2 or 3 of, independently, C₁-C₃ alkyl, C₁-C₃fluoroalkyl, C₁-C₃alkoxy, C₃fluoroalkoxy, halogen, cyano or nitro; phenylamino or phenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; diphenylamino or diphenylamino substituted by 1, 2 or 3 of, independently, C₁-C₃alkyl, C₁-C₃fluoroalkyl, C₁-C₃alkoxy, C₁-C₃fluoroalkoxy, halogen, cyano or nitro; or C₃-C₇cycloalkylamino, di(C₃-C₇cycloalkyl)amino, C₃-C₇cycloalkoxy, C₁-C₁₀fluoroalkoxy, C₁-C₅alkylamino or di(C₁-C₄alkyl)amino; or benzyloxy or phenoxy, wherein the benzyl and phenyl groups are in turn optionally substituted by 1, 2 or 3 of, independently, C₁-C₃alkyl, C₁-C₃fluoroalkyl, C₁-C₃alkoxy, C₁-C₃fluoroalkoxy, halogen, cyano or nitro; and
R^{h} is C₁-C₁₀alkyl, C₃-C₁₀alkenyl, C₃-C₁₀alkynyl, C₁-C₁₀fluoroalkyl, C₁-C₁₀cyanoalkyl, C₁-C₁₀nitroalkyl, C₂-C₁₀aminoalkyl, C₁-C₅alkylamino(C₁-C₅)alkyl, C₂-C₈dialkylamino(C₁-C₅)alkyl, C₃-C₇cycloalkyl(C₁-C₅)alkyl, C₁-C₅alkoxy(C₁-C₅)alkyl, C₃-C₅alkenyloxy(C₁-C₅)alkyl, C₃-C₅alkynyloxy(C₁-C₅)alkyl, C₁-C₅alkylthio(C₁-C₅)alkyl, C₁-C₅alkylsulfinyl(C₁-C₅)alkyl, C₅alkylsulfonyl(C₁-C₅)alkyl, C₂-C₈alkylideneaminoxy(C₁-C₅)alkyl, C₁-C₅alkylcarbonyl(C₁-C₅)alkyl, C₁-C₆alkoxycarbonyl(C₁-C₅)alkyl, aminocarbonyl(C₁-C₅)alkyl, C₅alkylaminocarbonyl(C₁-C₅)alkyl, C₂-C₈dialkylaminocarbonyl(C₁-C₅)alkyl, C₅alkylcarbonylamino(C₁-C₅)alkyl, N—(C₁-C₅)alkylcarbonyl-N—(C₁-C₅)alkylamino(C₁-C₅)alkyl, C₃-C₆trialkylsilyl(C₁-C₅)alkyl, phenyl(C₁-C₅)alkyl (wherein the phenyl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), heteroaryl(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₁-C₃alkylthio, C₁-C₃alkylsulfinyl, C₁-C₃ alkylsulfonyl, halogen, cyano or nitro), phenoxy(C₁-C₅)alkyl (wherein the phenyl is optionally substituted by 1, 2 or 3 of, independently, C₁-C₃alkyl, C₁-C₃fluoroalkyl, C₃alkoxy, C₁-C₃fluoroalkoxy, C₁-C₃alkylthio, C₁-C₃alkylsulfinyl, C₁-C₃ alkylsulfonyl, halogen, cyano or nitro), heteroaryloxy(C₁-C₅)alkyl (wherein the heteroaryl is optionally substituted by 1, 2 or 3 of, independently, C₁-C₃alkyl, C₁-C₃fluoroalkyl, C₁-C₃alkoxy, C₁-C₃fluoroalkoxy, C₃alkylthio, C₁-C₃alkylsulfinyl, C₁-C₃ alkylsulfonyl, halogen, cyano or nitro), C₃-C₅fluoroalkenyl, C₃-C₈cycloalkyl; phenyl or phenyl substituted by 1, 2 or 3 of, independently, C₁-C₃alkyl, C₁-C₃fluoroalkyl, C₁-C₃alkoxy, C₁-C₃fluoroalkoxy, halogen, cyano or nitro; heteroaryl or heteroaryl substituted by 1, 2 or 3 of, independently, C₁-C₃alkyl, C₃fluoroalkyl, C₁-C₃alkoxy, C₁-C₃fluoroalkoxy, halogen, cyano or nitro; C₁-C₆alkyl-C(O)—; or phenyl-C(O)— wherein the phenyl is optionally substituted by 1 or 2 of, independently, C₁-C₂alkyl, C₁fluoroalkyl, C₁-C₂alkoxy, C₁fluoroalkoxy, fluorine, chlorine, bromine, cyano or nitro;
wherein “heteroaryl” means an aromatic ring system containing at least one ring heteroatom and consisting either of a single ring or of two fused rings;
and wherein the compound of formula (I) is optionally present as an agrochemically acceptable salt thereof.

2. A compound as claimed in claim 1, wherein G is hydrogen; an agriculturally acceptable metal, or an agriculturally acceptable sulfonium or ammonium group; or G is —C(X^{a})—R^{a} or —C(X^{b})—X^{c}—R^{b}, wherein X^{a}, R^{a}, X^{b}, X^{c} and R^{b} are as defined herein.

3. A compound as claimed in claim 1, wherein G is hydrogen, —C(X^{a})—R^{a} or —C(X^{b})—X^{c}—R^{b}.

4. A compound as claimed in claim 1, wherein, when G is —C(X^{a})—R^{a} or —C(X^{b})—X^{c}—R^{b}, then X^{a}, X^{b} and X^{c} are oxygen, R^{a} is C₁-C₁₀alkyl, C₂-C₆alkenyl, C₂-C₆alkynyl, C₃-C₆cycloalkyl or C₁-C₄alkoxyC₁-C₄alkyl; and R^{b} is C₁-C₁₀alkyl, C₂-C₅alkenyl-CH₂—, C₂-C₄alkenyl-CH(Me)-, C₂-C₅alkynyl-CH₂—, C₂-C₄alkynyl-CH(Me)-, C₃-C₆cycloalkyl or C₁-C₄alkoxyC₁-C₄alkyl.

5. A compound as claimed in claim 1, or wherein R¹ is methyl.

6. A compound as claimed in claim 1, or wherein R¹ is methoxy.

7. A compound as claimed in claim 1, wherein, when R² is C₁-C₂alkoxy-C₁-C₃alkoxy- or C₁fluoroalkoxy-C₁-C₃alkoxy-, then R² is R^{2A}O—CH(R^{2B})—CH(R^{2C})—O—;
wherein R^{2A} is C₁-C₂alkyl or C₁fluoroalkyl; and
R^{2B} and R^{2C} are independently hydrogen or methyl, provided that one or both of R^{2B} and R^{2C} are hydrogen.

8. A compound as claimed in claim 1, wherein R² is hydrogen, methyl, ethyl, n-propyl, n-butyl, cyclopropyl, ethynyl, cyano, methoxy, ethoxy, C₁fluoroalkoxy, C₁fluoroalkylmethoxy, (difluoro)vinyloxy, or MeO—CH₂—CH₂—O—.

9. A compound as claimed in claim 1, wherein when R¹ is methyl then R² is not hydrogen.

10. A compound as claimed in claim 1, wherein R¹ is methyl or methoxy, and R² is methyl, ethyl, ethynyl, methoxy, ethoxy, C₁fluoroalkoxy, C₁fluoroalkylmethoxy, or MeO—CH₂—CH₂—O—;
or R¹ is methoxy and R² is hydrogen.

11. A compound as claimed in claim 1, wherein R¹ is methyl or methoxy, and R² is methyl, ethyl, methoxy or ethoxy; or R¹ is methoxy and R² is hydrogen.

12. A compound as claimed in claim 1, wherein R¹ is methyl and R² is methyl; or R¹ is methoxy and R² is hydrogen or methoxy.

13. A compound as claimed in claim 1, wherein R¹ is methyl, and R² is methyl, ethyl, methoxy or ethoxy.

14. A compound as claimed in claim 1, wherein R¹ is methyl, and R² is methyl.

15. A compound as claimed in claim 1, wherein R¹ is methoxy, and R² is hydrogen, methyl, ethyl, methoxy or ethoxy.

16. A compound as claimed in claim 1, wherein R¹ is methoxy, and R² is hydrogen or methoxy.

17. A compound as claimed in claim 1, wherein R⁵ and R⁶ are not taken together to be —(CH₂)ₙ₁— or —(CH₂)ₙ₂—X¹—(CH₂)ₙ₃—.

18. (canceled)

19. A compound as claimed in claim 1, wherein R⁴ and R⁵, independently of each other, are: hydrogen, C₁-C₃alkyl, R⁹—C≡C—C(R⁷)(R⁸)—, or Het-CH₂—; provided that when X is NR⁴, then at least one of R³ and R⁴ is hydrogen or C₁-C₃alkyl; and provided that when X is CR⁵R⁶, then at least one of R³ and R⁵ is hydrogen or C₁-C₃alkyl;
or R³ and R⁴ are taken together, and/or R³ and R⁵ are taken together, to form a chain which is (a), (b), (c), (d), (e), (f) or (g), as defined in any preceding claim.

20. (canceled)

21. A compound as claimed in claim 1, wherein X is NR⁴.

22-26. (canceled)

27. A method of controlling weeds in crops of useful plants, comprising applying a compound of formula (I), as defined in claim 1, or a herbicidal composition comprising such a compound, to the plants or to the locus thereof.

28. (canceled)
